(19) United States
(12) Patent Application Publication
(10) Pub. No.: US 2025/0136577 A1
(43) Pub. Date: May 1, 2025

(54) HETEROCYCLIC DERIVATIVES AS MALT1 INHIBITORS

(71) Applicant: C4X Discovery Limited, Manchester (GB)

(72) Inventors: Ian Duncan Linney, London (GB); Nicholas Charles Ray, London (GB); Martin John Watson, Manchester (GB); Paul Matthew Blaney, Manchester (GB); Oliver Thomas Kern, London (GB); Simon Osborne, London (GB); Martin Amber, London (GB); Chido Mpamhanga, London (GB)

(21) Appl. No.: 18/835,599

(22) PCT Filed: Feb. 3, 2023
(86) PCT No.: PCT/GB2023/050245

§ 371 Date: Aug. 2, 2024

(30) Foreign Application Priority Data
Feb. 3, 2022 (GB) 2201389.0
Nov. 29, 2022 (GB) 2217955.0

(51) Int. Cl. C07D 401/14; A61K 31/444; A61K 31/4709; A61K 31/498; A61K 31/4985; A61K 31/538; A61K 31/5383; A61K 31/5386; C07D 413/14; C07D 417/14; C07D 471/04; C07D 471/14; C07D 498/04; C07D 498/20

(52) U.S. Cl. CPC C07D 401/14; A61K 31/444; A61K 31/4709; A61K 31/498; A61K 31/4985; A61K 31/538; A61K 31/5383; A61K 31/5386; C07D 413/14; C07D 417/14; C07D 471/04; C07D 471/14; C07D 498/04; C07D 498/20

(57) ABSTRACT

The present invention relates to compounds that are MALT1 inhibitors. The compounds have the structural formula I defined herein. The present invention also relates to processes for the preparation of these compounds, to pharmaceutical compositions comprising them, and to their use in the treatment of diseases or disorders associated with MALT1.

### INTRODUCTION

The present invention relates to compounds that are MALT1 inhibitors. The present invention also relates to processes for the preparation of these compounds, to pharmaceutical compositions comprising them, and to their use in the treatment of diseases or disorders associated with MALT1.

### BACKGROUND OF THE INVENTION

Mucosa-associated lymphoid tissue lymphoma translocation protein 1 (MALT1) is an intracellular protein which plays a key role in antigen receptor-induced NF-κB pathway activation in T and B lymphocytes, via both a scaffolding function and a protease function. Antigen receptor triggering leads to formation of the CBM complex, comprising a CARMA or CARD protein, BCL10 and MALT1, that subsequently acts as a scaffold to recruit the ubiquitin ligase TRAF6 and the kinases TAK1 and IKK. This leads to NF-κB activation through IKK-dependent phosphorylation and proteasomal degradation of the NF-κB inhibitor IkB, allowing NF-κB to translate to the nucleus and initiate transcription of target genes (doi.org/10.1038/ni1568; doi: 10.1007/s00018-015-2059-z). Additionally, MALT1 proteolytically cleaves a variety of substrates involved in NF-κB pathway regulation, including RelB, A20, CYLD, regnase-1, HOIL, BCL10 and NIK; as well as autoproteolytic cleavage (doi: 10.1016/j.biochi.2015.09.018). The overall effect of these cleavage events is thought to be expansion of the amplitude and duration of the NF-κB response (doi: 10.1016/j.biochi.2015.09.018).

Several lines of genetic evidence suggest a key role of MALT1 in the immune response. Mice lacking MALT1 protein were viable, but showed impairment in the generation and activation of T_{reg} cells and less activated T cells in the periphery. MALT1 KO mice were protected in inflammatory models for MS (EAE) and rheumatoid arthritis (doi: 10.4049/jimmunol. 1201351). Mice expressing proteolytically inactive MALT1 show defects in multiple immune cell types including mature T- and B-cells and T_{reg} cells, and develop progressive multiorgan inflammatory pathology (doi: 10.4049/jimmunol. 1402254; doi: 10.3389/fimmu.2020.00745). A small cohort of human patients with defective MALT1 expression and/or function have presented with combined immunodeficiency (doi: 10.1007/s10875-014-0125-1; doi: 10.1016/j.jaci.2013.04.047).

The highly aggressive activated B-cell (ABC) subtype of diffuse large B-cell lymphoma (DLBCL) is dependent on NF-κB activation via constitutive activation of the CARMA1-BCL10-MALT1 pathway for its survival and proliferation (doi.org/10.1038/nature04687). This constitutive NF-κB activation occurs often due to a variety of oncogenic mutations in pathway genes, including CARD11, MYD88, CD79A/B and A20 (doi: 10.1038/nature07968; doi: 10.1016/j.ccr.2012.11.003). Pre-clinical studies have suggested that MALT1 protease inhibition may be an effective treatment rationale for ABC-DLBCL, as treatment with the MALT1 inhibitor z-VRPR-fmk decreased the expression of NF-κB target genes with concomitant reduction in cell growth and viability (doi: 10.1073/pnas.0907511106; doi: 10.1084/jem.20091167), and small-molecule MALT1 inhibitors have also been shown to be active in xenograft models of ABC-DLBCL (doi: 10.1016/j.ccr.2012.11.002; doi: 10.1016/j.ccr.2012.11.003). Chronic activation of the BCR-mediated CBM-NF-κB pathway has been identified in a subset of mantle cell lymphoma (MCL) lines, suggesting that a subset of MCL may also be responsive to MALT1 inhibition (doi: 10.1038/nm.3435). In MALT lymphoma (a type of non-Hodgkin lymphoma (NHL), the fusion protein clAP2-MALT1 leads to constitutive activation of the NF-κB pathway, and these patients may also benefit from treatment with MALT1 inhibitors (doi 10.1074/jbc.M605116200). MALT1 inhibition may play a role in the treatment of some solid tumour types such as glioblastoma, breast cancer, melanoma, lung cancer, prostate cancer, pancreatic cancer and osteosarcoma (doi: 10.1038/s41388-019-0958-4; doi: 10.15252/embj.2019102030; doi: 10.1111/jcmm. 15383; doi: 10.1038/oncsis.2017.68; doi: 10.1038/onc.2015.146; doi: 10.3390/biomedicines9030250; doi: 10.1002/ijc.32567).

BTK inhibitors such as ibrutinib are important therapies for cancers such as MCL and chronic lymphocytic leukemia (CLL), but effectiveness is limited due to primary or acquired resistance (doi: 10.3390/cancers12051328). MALT1 sits downstream of BTK in the NF-κB activation pathway and therefore may be an effective target either in combination with BTK inhibitors, or in BTKi-refractive tumours. MALT1 has been shown to be constitutively active in CLL cell lines and treatment with the MALT-1 inhibitor MI-2 is effective against both naïve and ibrutinib-resistant cell lines (doi: 10.1158/0008-5472.CAN-17-2485). MALT1 inhibition has also been shown to be synergistic with the mTORC1 inhibitor, rapamycin, in ABC-DLBCL cell lines, PDX and in vivo models, opening further possibilities for combination treatment and mitigation strategies for MALT1i resistance (doi: 10.1182/blood.2019004713).

MALT1 inhibitors have also been proposed to be effective therapies for a range of cancers, independent of dysregulation of the NF-κB pathway, as an immunomodulatory therapy (doi: 10.1038/s41586-019-1215-2; WO2018/226150 & WO2018/141749). Genetic evidence from MALT1-deficient mice suggests that MALT1 promotes development of T_{reg} cells in vivo, which in turn inhibit several types of immune cells, suppressing the anti-tumour immune response. Further studies targeting the CBM complex (by either MALT1 inhibition or CARMA1 deletion in T_{reg} cells) led to a gain of effector activity by T_{reg} cells and enhanced control of tumour growth. A MALT1 inhibitor synergized with anti-PD1 therapy in both poorly immunogenic and immunogenic murine melanoma models (doi: 10.1038/s41586-019-1215-2), suggesting a possible combinatorial role of MALT1 inhibitors with immunotherapies, including anti-PD1, anti-PD-L1 and anti-CTLA4.

As well as having a use in oncology indications, small-molecule MALT1 inhibitors may also be effective therapies in inflammatory disorders, for example multiple sclerosis, psoriasis, ulcerative colitis and rheumatoid arthritis. MI-2 has been shown to suppress the differentiation of monocytes into osteoclasts in the presence or absence of TNFa, and to ameliorate the pathologic bone erosion and synovitis in a mouse collagen-induced arthritis (CIA) model, suggesting a role for MALT1 inhibitors in the treatment of rheumatoid arthritis (doi: 10.1038/s41598-017-12349-9). The scaffold protein CARD14 forms a signalling complex with BCL10 and MALT1 in keratinocytes and this process is enhanced upon pathogenic CARD14 mutation which as in turn been linked to susceptibility to psoriasis (doi: 10.1016/j.jid.2016.09.031). MALT1 inhibitors have also been successfully tested in mouse models of multiple sclerosis and ulcerative colitis (doi: 10.1186/1742-2094-11-124). Pharmacological inhibition of MALT1 protease activity has been shown to suppress endothelial activation through increasing MCPIP1 expression, inhibiting TNFa-induced VCAM-1 expression in HUVECs and LPS-induced VCAM-1 expression in mice, suggesting a possible role for MALT1 inhibitors in the treatment of vascular inflammatory diseases such as atherosclerosis (doi: 10.1016/j.cellsig.2018.05.009).

Homozygous MALT1 W580S mutations have been determined in patients with combined immunodeficieny (CID). This was associated with low MALT1-W580S protein levels leading to reduced amplitude but extended duration of NF-κB due to impaired negative feedback through reduced HOIL1 cleavage, manifesting clinically in increased dermal and GI inflammation and increased susceptibility to infection (doi.org/10.1016/j.jaci.2013.10.045). Small-molecule ligands known to bind to the MALT1 wild-type allosteric site have been shown to also bind to the W580S mutant leading to enzyme stabilization and reconstitution of protein levels, even after ligand washout. Both scaffolding and protease functions were restored; thus allosteric MALT1 inhibitors may be able to serve as ‘molecular correctors’ in cases of W80S MALT1 mutations (doi.org/10.1038/s41589-018-0222-1).

Clinical and pre-clinical studies are ongoing with a variety of MALT1 inhibitors. JNJ-67856633 is a small molecule MALT1 inhibitor currently in Phl studies in patients with NHL and CLL. A review of MALT1 inhibitor patents has recently been published (doi: 10.1080/13543776.2021.1951703).

Therefore, there is an ongoing need for agents capable of MALT1 inhibition, given the role of MALT1 in multiple indications.

### SUMMARY OF THE INVENTION

In one aspect, the present invention provides a compound, or a pharmaceutically acceptable salt thereof as defined herein.

In another aspect, the present invention provides a pharmaceutical composition comprising a compound of the invention as defined herein, or a pharmaceutically acceptable salt thereof, and one or more pharmaceutically acceptable excipients.

In another aspect, the present invention relates to a compound of the invention as defined herein, or a pharmaceutically acceptable salt thereof, or a pharmaceutical composition as defined herein, for use in therapy.

In another aspect, the present invention relates to a compound of the invention as defined herein, or a pharmaceutically acceptable salt thereof, or a pharmaceutical composition as defined herein, for use in the treatment of diseases or disorders mediated by MALT1.

In another aspect, the present invention relates to a method of treating a disease or disorder mediated by MALT1, said method comprising administering to a subject in need of such treatment a therapeutically effective amount of a compound of the invention as defined herein, or a pharmaceutically acceptable salt thereof, or a pharmaceutical composition as defined herein.

Examples of diseases or disorders mediated by MALT1 include:
i) lymphomas, leukaemias, carcinomas, and sarcomas; such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, or GIST (gastrointestinal stromal tumour); and
ii) immunological diseases including autoimmune and inflammatory disorders; such as arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, 20 berylliosis, or polymyositis.

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukemia (CLL), or small lymphocytic lymphoma (SLL), said method comprising administering to a subject in need of such treatment a therapeutically effective amount of a compound, or a pharmaceutically acceptable salt thereof, or a pharmaceutical composition as defined herein.

The present invention further provides a method of synthesising a compound, or a pharmaceutically acceptable salt thereof, as defined herein.

In another aspect, the present invention provides a compound, or a pharmaceutically acceptable salt thereof, obtainable by, or obtained by, or directly obtained by a method of synthesis as defined herein.

In another aspect, the present invention provides novel intermediates as defined herein which are suitable for use in any one of the synthetic methods set out herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

In this specification the term “alkylene” includes both straight and branched chain divalent alkyl groups. For example, “C₁₋₄alkylene” includes methylene (—CH₂—), ethylene (—CH₂CH₂—), propylene and butylene.

In this specification the term “alkoxy” includes both straight and branched chain alkyl groups singularly bonded to oxygen. For example, “C₁₋₄alkoxy” includes methoxy, ethoxy, iso-propoxy and t-butoxy.

The term “(m-nC)” or “(m-nC) group” used alone or as a prefix, refers to any group having m to n carbon atoms.

“Cycloalkyl” means a hydrocarbon monocyclic or bicyclic ring containing carbon atoms. Examples of monocyclic cycloalkyl groups include cyclopropyl, cyclobutyl, cyclopentyl, cyclohexyl, cycloheptyl. Bicyclic rings may be fused or spiro attached; examples of bicyclic cycloalkyl groups include bicyclo[2.2.2]octane, bicyclo[2.1.1]hexane, bicyclo[1.1.1]pentane, spiro[2.4] heptane, bicyclo[4.1.0]heptane and bicyclo[2.2.1]heptane.

The term “halo” refers to fluoro, chloro, bromo and iodo.

The term “haloalkyl” is used herein to refer to an alkyl group respectively in which one or more hydrogen atoms have been replaced by halogen (e.g. fluorine) atoms. Examples of haloalkyl groups include fluoroalkyl groups such as —CHF₂,—CH₂CF₃, or perfluoroalkyl/alkoxy groups such as —CF₃, or —CF₂CF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, dihydroisoxazolyl (such as 4,5-dihydroisoxazolyl), dihydropyridinyl (such as 1,2-dihydropyridinyl or 1,6-dihydropyridinyl), tetrahydro-oxazolyl, tetrahydro-oxadiazolyl, tetrahydro-dioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1, 1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl.

As the skilled person would appreciate, any heterocycle may be linked to another group via any suitable atom, such as via a carbon or nitrogen atom. Suitably, the term “heterocyclyl”, “heterocyclic” or “heterocycle” will refer to 4, 5, 6 or 7 membered monocyclic rings as defined above.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Suitably, the term “heteroaryl” or “heteroaromatic” will refer to 5 or 6 membered monocyclic heteroaryl rings as defined above.

Non-limiting examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl, 4,5,6,7-tetrahydrobenzo[d] isoxazolyl, 4,5,6,7-tetrahydro-[1,2,3]triazolo[1,5-a]pyridinyl, 5,6-dihydro-8H-[1,2,4]triazolo[3,4-c][1,4]oxazinyl, 5,6-dihydro-4H-pyrrolo[1,2-c][1,2,3]triazolyl, 6,7-dihydro-5H-pyrrolo[2, 1-c][1,2,4]triazolyl, 5,6,7,8-tetrahydro-[1,2,4]triazolo[4,3-a]pyridinyl, 6,7-dihydro-4H-[1,2,3]triazolo[5,1-c][1,4]oxazinyl and 1,4,5,6-tetrahydrocyclopenta [d][1,2,3]triazol-5-yl.

Non-limiting examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Non-limiting examples of six membered heteroaryl groups include but are not limited to pyridyl, pyrazinyl, pyridazinyl, pyrimidinyl and triazinyl.

Particular non-limiting examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular non-limiting examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Particular non-limiting examples of bicyclic heteroaryl groups containing a five membered ring fused to a five membered ring include but are not limited to 6,7-dihydro-5H-pyrrolo[2, 1-c][1,2,4]triazolyl, 5,6-dihydro-4H-pyrrolo[1,2-c][1,2,3]triazolyl and 1,4,5,6-tetrahydrocyclopenta [d][1,2,3]triazol-5-yl.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In this particular embodiment, an aryl is phenyl or naphthyl, especially phenyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention provides a compound of Formula I, or a pharmaceutically acceptable salt thereof:

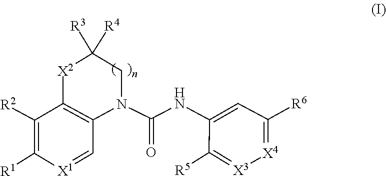

wherein:
X¹ is N or CH;
X² is O, NR⁷, or CR⁸R⁹;
X³ is N or CH;
X⁴ is N or CR¹⁰;
provided that X³ and X⁴ cannot both be N;
R¹ is selected from hydrogen, C₁₋₄alkyl, hydroxy, C₁₋₄alkoxy, C₁₋₄haloalkyl, halo, NR¹¹R¹², C₃₋₇cycloalkyl, and O—C₃₋₇cycloalkyl;
R² is selected from hydrogen, C₁₋₄alkyl, C₁₋₄haloalkyl, C₃₋₇cycloalkyl, halo, cyano, NR¹³R¹⁴, C(O)OR¹⁵, C(O)NR¹⁶R¹⁷, OR¹⁸, 4- to 7-membered heterocyclyl, 5- or 6-membered heteroaryl, and phenyl, wherein said 4- to 7-membered heterocyclyl, 5- or 6-membered heteroaryl, and phenyl are optionally substituted with one or more substituents selected from C₁₋₄alkyl, halo, and cyano;
R³ and R⁴ are independently selected from hydrogen, C₁₋₄alkyl, C₂₋₄alkenyl, C₂₋₄alkynyl, C₁₋₄haloalkyl, C₃₋₇cycloalkyl, hydroxy, C₁₋₄alkoxy, C₁₋₄haloalkoxy, halo, cyano, NR¹⁹R²⁰, 4- to 7-membered heterocyclyl, 5- or 6-membered heteroaryl, and phenyl, wherein said C₁₋₄alkyl, 4- to 7-membered heterocyclyl, 5- or 6-membered heteroaryl, and phenyl are optionally substituted with one or more substituents selected from C₁₋₄alkyl, hydroxy, C₁₋₄alkoxy, halo, and cyano; OR
R³ and R⁴, together with the carbon atom to which they are attached, form an oxo group, a 4- to 7-membered heterocyclic ring, or a 3- to 6-membered cycloalkyl ring;
R⁵ is selected from hydrogen, C₁₋₄alkyl, C₁₋₄alkoxy, and C₁₋₄haloalkyl;
R⁶ is selected from hydrogen, halo, C₁₋₄alkyl, C₁₋₄alkoxy, cyano, and C₁₋₄haloalkyl;
R⁷ is hydrogen, C₁₋₄alkyl, C₃₋₇cycloalkyl, 5- or 6-membered heteroaryl or 4- to 7-membered heterocyclyl, wherein said C₁₋₄alkyl, C₃₋₇cycloalkyl, 5- or 6-membered heteroaryl, and 4- to 7-membered heterocyclyl are optionally substituted with one or more substituents selected from C₁₋₄alkyl, C₁₋₄alkoxy, halo, and cyano; OR
R⁷ and R³, together with the atoms to which they are attached, form a 5- or 6-membered heterocyclic ring, optionally substituted with one or more substituents selected from C₁₋₄alkyl, C₁₋₄alkoxy, halo, and cyano;
R⁸ and R⁹ are independently selected from hydrogen, C₁₋₄alkyl, C₂₋₄alkenyl, C₂₋₄alkynyl, C₁₋₄haloalkyl, C₃₋₇cycloalkyl, hydroxy, C₁₋₄alkoxy, C₁₋₄haloalkoxy, halo, cyano, NR²¹R²², 4- to 7-membered heterocyclyl, 5- or 6-membered heteroaryl, and phenyl, wherein said C₁₋₄alkyl, 4- to 7-membered heterocyclyl, 5- or 6-membered heteroaryl, and phenyl are optionally substituted with one or more substituents selected from C₁₋₄alkyl, C₁₋₄alkoxy, halo, and cyano; OR
R⁸ and R⁹, together with the carbon atom to which they are attached, form a 4- to 7-membered heterocyclic ring, or a 3- to 6-membered cycloalkyl ring;
R¹⁰ is selected from C(O)NR²³R²⁴, C₁₋₄haloalkoxy, C₁₋₄haloalkyl, C₁₋₄alkoxy, C₃₋₇cycloalkyl, 4- to 7-membered heterocyclyl, and 5- or 6-membered heteroaryl, wherein said C₃₋₇cycloalkyl, 4- to 7-membered heterocyclyl, and 5- or 6-membered heteroaryl groups are optionally substituted with one or more substituents selected from C₁₋₄alkyl, C₁₋₃alkylene-R²⁵, hydroxy, NR²⁶R²⁷, and halo;
R¹¹ and R¹² are independently selected from hydrogen, C₁₋₄alkyl, C₃₋₇cycloalkyl, and C₁₋₄haloalkyl; or R¹¹ and R¹², together with the nitrogen atom to which they are attached, form a 4- to 7-membered heterocyclic ring;
R¹³ and R¹⁴ are independently selected from hydrogen, C₁₋₄alkyl, C₃₋₇cycloalkyl, and C₁₋₄haloalkyl; or R¹³ and R¹⁴, together with the nitrogen atom to which they are attached, form a 4- to 7-membered heterocyclic ring;
R¹⁵ is hydrogen or C₁₋₄alkyl;
R¹⁶ and R¹⁷ are independently selected from hydrogen, C₁₋₄alkyl, C₃₋₇cycloalkyl, and C₁₋₄haloalkyl; or R¹⁶ and R¹⁷, together with the nitrogen atom to which they are attached, form a 4- to 7-membered heterocyclic ring;
R¹⁸ is selected from hydrogen, C₁₋₄alkyl, C₁₋₃alkylene-R²³, C₁₋₄haloalkyl, C₃₋₇cycloalkyl, 4- to 7-membered heterocyclyl, and 5- or 6-membered heteroaryl;
R¹⁹, R²⁰, R²¹, and R²² are independently selected from hydrogen and C₁₋₄alkyl;
R²³ and R²⁴ are independently selected from hydrogen, C₁₋₄alkyl and C₃₋₇cycloalkyl;
R²⁵ is selected from OH, C₁₋₄alkoxy, C₃₋₇cycloalkyl, NR²⁸R²⁹, C(O)OR³⁰, and C(O)NR³¹R³²;
R²⁶, R²⁷, R²⁸, R²⁹, R³⁰, R³¹, and R³² are independently selected from hydrogen and C₁₋₄alkyl; and
n is 0, 1, or 2.

Particular compounds of the invention include, for example, compounds of the formula I, or pharmaceutically acceptable salts thereof, wherein, unless otherwise stated, each of X¹, X², X³, X⁴, R¹, R², R³, R⁴, R⁵, R⁶, R⁷, R⁸, R⁹, R¹⁰, R¹¹, R¹², R¹³, R¹⁴, R¹⁸, R²³, R²⁴, and n has any of the meanings defined hereinbefore or in any of paragraphs (1) to (94) hereinafter. For the avoidance of doubt, the scope of the present invention encompasses compounds of formula I, or pharmaceutically acceptable salts thereof, wherein any of the substituent definitions defined herein may be combined with any of the other substituent definitions also defined herein:
(1) X¹ is N;
(2) X¹ is CH;
X² is O or NR⁷;
(3)
(4) X² is O;
(5) X² is NR⁷;
(6) X² is CR⁸R⁹;
(7) X³ is N;
(8) X³ is CH;
(9) X⁴ is N;
(10) X⁴ is CR¹⁰;
(11) One of X³ and X⁴ is N, provided that X³ and X⁴ cannot both be N;
(12) X³ is N and X⁴ is CR¹⁰;
(13) R¹ is selected from hydrogen, C₁₋₄alkyl, hydroxy, C₁₋₄alkoxy, C₁₋₄haloalkyl, halo, NR¹¹R¹², and C₃₋₇cycloalkyl;
(14) R¹ is selected from hydrogen, C₁₋₄alkyl, hydroxy, C₁₋₄alkoxy, and NR¹¹R¹²;
(15) R¹ is selected from hydrogen, C₁₋₄alkyl, and NR¹¹R¹²;
(16) R¹ is selected from hydrogen, methyl, and NH2;
(17) R¹ is selected from hydrogen and methyl;
(18) R¹ is hydrogen;
(19) R² is selected from hydrogen, C₁₋₄alkyl, C₁₋₄haloalkyl, C₃₋₇cycloalkyl, halo, cyano, OR¹⁸, 4- to 7-membered heterocyclyl, 5- or 6-membered heteroaryl, and phenyl, wherein said 4- to 7-membered heterocyclyl, 5- or 6-membered heteroaryl, and phenyl are optionally substituted with one or more substituents selected from C₁₋₄alkyl, halo, and cyano;
(20) R² is selected from hydrogen, C₁₋₄alkyl, C₃₋₇cycloalkyl, halo, cyano, OR¹⁸, 5- or 6-membered heteroaryl, and phenyl, wherein said 5- or 6-membered heteroaryl, and phenyl are optionally substituted with one or more substituents selected from C₁₋₄alkyl, halo, and cyano;
(21) R² is selected from hydrogen, C₃₋₇cycloalkyl, halo, cyano, OR¹⁸, 5- or 6-membered heteroaryl, and phenyl;
(22) R² is selected from hydrogen, cyclopropyl, halo, cyano, methoxy, 5-membered heteroaryl, and phenyl;
(23) R² is selected from halo, cyano, methoxy, 5-membered heteroaryl, and phenyl;
(24) R² is selected from halo, and cyano;
(25) R² is selected from chloro and bromo;
(26) R² is bromo;
(27) R³ and R⁴ are independently selected from hydrogen, C₁₋₄alkyl, C₂₋₄alkynyl, C₁₋₄haloalkyl, C₃₋₇cycloalkyl, hydroxy, C₁₋₄alkoxy, halo, cyano, 4- to 7-membered heterocyclyl, 5- or 6-membered heteroaryl, and phenyl;
(28) R³ and R⁴ are independently selected from hydrogen, C₁₋₄alkyl, C₂₋₄alkynyl, C₁₋₄haloalkyl, C₃₋₇cycloalkyl, C₁₋₄alkoxy, halo, and cyano;
(29) R³ and R⁴ are independently selected from hydrogen, C₁₋₄alkyl, C₂₋₄alkynyl, C₁₋₄haloalkyl, and cyano;
(30) R³ and R⁴ are independently selected from hydrogen, C₁₋₄alkyl, ethynyl, trifluoromethyl, and cyano;
(31) R³ and R⁴ are independently selected from hydrogen, methyl, ethyl, propyl, isopropyl, trifluoromethyl, and cyano;
(32) R³ is hydrogen or methyl and R⁴ is selected from hydrogen, methyl, ethyl, propyl, isopropyl, trifluoromethyl, and cyano;
(33) R³ is hydrogen and R⁴ is selected from hydrogen, methyl, and trifluoromethyl;
(34) R³ and R⁴ are both hydrogen;
(35) R³ is methyl and R⁴ is selected from methyl and cyano;
(36) R³ and R⁴ are both methyl;
(37) R³ and R⁴, together with the carbon atom to which they are attached, form a 4- to 7-membered heterocyclic ring, or a 3- to 6-membered cycloalkyl ring;
(38) R³ and R⁴, together with the carbon atom to which they are attached, form a 5- to 6-membered heterocyclic ring, or a 3- to 5-membered cycloalkyl ring;
(39) R³ and R⁴, together with the carbon atom to which they are attached, form a 5-membered heterocyclic ring;
(40) R¹ and R² cannot both be hydrogen;
(41) R² and R³ cannot both be hydrogen;
(42) R¹, R², and R³ cannot all be hydrogen;
(43) R¹, R², R³, and R⁴ cannot all be hydrogen;
(44) R⁵ is selected from hydrogen and C₁₋₄alkyl;
(45) R⁵ is selected from hydrogen and methyl;
(46) R⁵ is hydrogen;
(47) R⁶ is selected from halo, C₁₋₄alkyl, C₁₋₄alkoxy, cyano, and C₁₋₄haloalkyl;
(48) R⁶ is selected from halo, cyano, and C₁₋₄haloalkyl;
(49) R⁶ is selected from halo and C₁₋₄haloalkyl;
(50) R⁶ is selected from chloro, bromo and trifluoromethyl;
(51) R⁶ is selected from chloro and trifluoromethyl;
(52) R⁶ is chloro;
(53) R⁷ is hydrogen, C₁₋₄alkyl, or 4- to 7-membered heterocyclyl, wherein said C₁₋₄alkyl and 4- to 7-membered heterocyclyl are optionally substituted with one or more substituents selected from C₁₋₄alkyl, C₁₋₄alkoxy, halo, and cyano;
(54) R⁷ is hydrogen, C₁₋₄alkyl, or 4- to 7-membered heterocyclyl;
(55) R⁷ is hydrogen or C₁₋₄alkyl;
(56) R⁷ is hydrogen or methyl;
(57) R⁷ is C₁₋₄alkyl;
(58) R⁷ is methyl;
(59) R⁷ and R³, together with the atoms to which they are attached, form a 5- or 6-membered heterocyclic ring, optionally substituted with one or more substituents selected from methyl, methoxy, fluoro, chloro, and cyano;
(60) R⁷ and R³, together with the atoms to which they are attached, form a 5- or 6-membered heterocyclic ring;
(61) R⁷ and R³, together with the atoms to which they are attached, form a 5- or 6-membered heterocyclic ring;
(62) R⁷ and R³, together with the atoms to which they are attached, form a pyrrolidine, piperazine or piperidine ring;
(63) R⁷ and R³, together with the atoms to which they are attached, form a pyrrolidine or piperidine ring;
(64) R⁸ and R⁹ are independently selected from hydrogen, C₁₋₄alkyl, C₂₋₄alkenyl, C₂₋₄alkynyl, C₁₋₄haloalkyl, C₃₋₇cycloalkyl, C₁₋₄haloalkoxy, halo, cyano, NR²¹R²², 4- to 7-membered heterocyclyl, 5- or 6-membered heteroaryl, and phenyl, wherein said C₁₋₄alkyl, 4- to 7-membered heterocyclyl, 5- or 6-membered heteroaryl, and phenyl are optionally substituted with one or more substituents selected from C₁₋₄alkyl, C₁₋₄alkoxy, halo, and cyano;
(65) R⁸ and R⁹ are independently selected from hydrogen, C₁₋₄alkyl, C₁₋₄haloalkyl, C₃₋₇cycloalkyl, halo, and cyano;
(66) R⁸ and R⁹ are independently selected from hydrogen, C₁₋₄alkyl, and C₁₋₄haloalkyl;
(67) R⁸ and R⁹ are independently selected from hydrogen and C₁₋₄alkyl;
(68) R⁸ and R⁹ are independently selected from hydrogen and methyl;
(69) R⁸ and R⁹ are both hydrogen;
(70) R⁸ and R⁹, together with the carbon atom to which they are attached, form a 4- to 6-membered heterocyclic ring, or a 3- to 5-membered cycloalkyl ring;
(71) R⁸ and R⁹, together with the carbon atom to which they are attached, form a 3- to 5-membered cycloalkyl ring;
(72) R⁸ and R⁹, together with the carbon atom to which they are attached, form a cyclopropyl ring;
(73) R¹⁰ is selected from C(O)NR²³R²⁴, C₁₋₄haloalkoxy, C₃₋₇cycloalkyl, and 5- or 6-membered heteroaryl, wherein said C₃₋₇cycloalkyl, and 5- or 6-membered heteroaryl groups are optionally substituted with one or more substituents selected from C₁₋₄alkyl, C₁₋₃alkylene-R²⁵, hydroxy, NR²⁶R²⁷, and halo;
(74) R¹⁰ is selected from C(O)NR²³R²⁴, C₁₋₄haloalkoxy, 4- to 7-membered heterocyclyl, and 5- or 6-membered heteroaryl, wherein said 4- to 7-membered heterocyclyl and 5- or 6-membered heteroaryl groups are optionally substituted with one or more substituents selected from C₁₋₂alkyl, C₁₋₂alkylene-R²⁵, and halo;
(75) R¹⁰ is selected from C(O)NR²³R²⁴, C₁₋₂haloalkoxy, and 5- or 6-membered heteroaryl, wherein said 5- or 6-membered heteroaryl group is optionally substituted with one or more substituents selected from C₁₋₂alkyl, C₁₋₂alkylene-R²⁵, and halo;
(76) R¹⁰ is 5- or 6-membered heteroaryl, optionally substituted with one or more substituents selected from C₁₋₄alkyl, C₁₋₃alkylene-R²⁵, hydroxy, NR²⁶R²⁷, and halo;
(77) R¹⁰ is 5- or 6-membered heteroaryl, optionally substituted with one or more substituents selected from C₁₋₂alkyl and C₁₋₂alkylene-R²⁵;
(78) R¹⁰ is triazolyl, optionally substituted with one or more substituents selected from C₁₋₂alkyl and C₁₋₂alkylene-R²⁵;
(79) R¹⁰ is triazolyl, optionally substituted with one or more substituents selected from methyl and CH₂-R²⁵;
(80) R¹⁰ is 2H-1,2,3-triazol-2-yl, optionally substituted with one or more substituents selected from C₁₋₂alkyl and C₁₋₂alkylene-R²⁵.
(81) R¹⁰ is 2H-1,2,3-triazol-2-yl, optionally substituted with one or more substituents selected from methyl and CH₂-R²⁵,
(82) R¹⁰ is 2H-1,2,3-triazol-2-yl;
(83) R¹¹ and R¹² are independently selected from hydrogen and methyl;
(84) R¹¹ and R¹² are both hydrogen;
(85) R¹³ and R¹⁴ are independently selected from hydrogen and methyl;
(86) R¹³ and R¹⁴ are both hydrogen;
(87) R¹⁸ is selected from hydrogen and C₁₋₄alkyl;
(88) R¹⁸ is C₁₋₄alkyl;
(89) R¹⁸ is methyl;
(90) R²³ and R²⁴ are independently selected from hydrogen, methyl and cyclopropyl; such as from hydrogen and methyl; or from methyl and cyclopropyl;
(91) R²³ and R²⁴ are both hydrogen;
(92) n is 0 or 1;
(93) n is 0;
(94) n is 1;

Suitably, X¹ is as defined in any one of paragraphs (1) to (2) above. Preferably, X¹ is as defined in paragraph (1) above.

Suitably, X² is as defined in any one of paragraphs (3) to (6) above. Preferably, X² is as defined in paragraph (3) above.

Suitably, X³ is as defined in any one of paragraphs (7) to (8) above. Preferably, X³ is as defined in paragraph (7) above.

Suitably, X⁴ is as defined in any one of paragraphs (9) to (10) above. Preferably, X⁴ is as defined in paragraph (10) above.

Suitably, X³ and X⁴ are as defined in any one of paragraphs (11) to (12) above. Preferably, X³ and X⁴ are as defined in paragraph (12) above.

Suitably, R¹ is as defined in any one of paragraphs (13) to (18) above. In an embodiment, R¹ is as defined in paragraph (18) above.

Suitably, R² is as defined in any one of paragraphs (198) to (26) above. In an embodiment, R² is as defined in paragraph (26) above.

Suitably, R³ and R⁴ are as defined in any one of paragraphs (27) to (39) above. In an embodiment, R³ and R⁴ are as defined in paragraphs (33) to (36) above.

Suitably, R¹ to R⁴ are as defined in any one of paragraphs (40) to (43) above.

Suitably, R⁵ is as defined in any one of paragraphs (44) to (46) above. In an embodiment, R⁵ is as defined in paragraph (46) above.

Suitably, R⁶ is as defined in any one of paragraphs (47) to (52) above. In an embodiment, R⁶ is as defined in any one of paragraphs (51) to (52) above.

Suitably, R⁷ is as defined in any one of paragraphs (53) to (58) above. In an embodiment, R⁷ is as defined in paragraph (58) above.

Suitably, R⁷ and R³ are as defined in any one of paragraphs (59) to (63) above. In an embodiment, R⁷ and R³ are as defined in paragraph (63) above.

Suitably, R⁸ and R⁹ are as defined in any one of paragraphs (64) to (72) above. In an embodiment, R⁸ and R⁹ are as defined in any one of paragraphs (67) to (69) above.

Suitably, R¹⁰ is as defined in any one of paragraphs (73) to (82) above. In an embodiment, R¹⁰ is as defined in paragraph (82) above.

Suitably, R¹¹ and R¹² are as defined in any one of paragraphs (83) to (84) above. In an embodiment, R¹¹ and R¹² are as defined in paragraph (84) above.

Suitably, R¹³ and R¹⁴ are as defined in any one of paragraphs (85) to (86) above.

Suitably, R¹⁸ is as defined in any one of paragraphs (87) to (89) above. In an embodiment, R¹⁸ is as defined in paragraph (89) above.

Suitably, R²³ and R²⁴ are as defined in any one of paragraphs (90) to (91) above. In an embodiment, R²³ and R²⁴ are as defined in paragraph (91) above.

Suitably, n is as defined in any one of paragraphs (92) to (94) above. In an embodiment, n is as defined in paragraph (94) above.

Conveniently, the compound of Formula (I) has one of the sub-structural formulae (IA) to (IJ) described hereinafter.

In a further group of compounds, the compounds have the structural formula IA shown below:

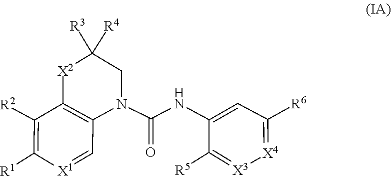

wherein X¹, X², X³, X⁴, R¹, R², R³, R⁴, R⁵, and R⁶ are as defined hereinbefore.

In a further group of compounds, the compounds have the structural formula IA shown above, wherein X¹ is as defined in any one of paragraphs (1) to (2); X² is as defined in any one of paragraphs (3) to (6); X³ is as defined in any one of paragraphs (7) to (8); X⁴ is as defined in any one of paragraphs (9) to (10); R¹ is as defined in any one of paragraphs (13) to (18) above; R² is as defined in any one of paragraphs (19) to (26) above; R³ and R⁴ are as defined in any one of paragraphs (27) to (39) above; R⁵ is as defined in any one of paragraphs (44) to (46) above; and R⁶ is as defined in any one of paragraphs (47) to (52) above.

In a further group of compounds, the compounds have the structural formula IB shown below:

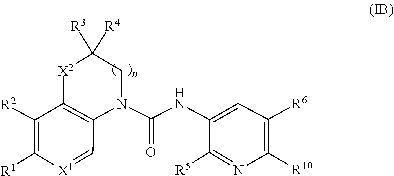

wherein X¹, X², R¹, R², R³, R⁴, R⁵, R⁶, R¹⁰ and n are as defined hereinbefore.

In a further group of compounds, the compounds have the structural formula IB shown above, wherein X¹ is as defined in any one of paragraphs (1) to (2); X² is as defined in any one of paragraphs (3) to (6); R¹ is as defined in any one of paragraphs (13) to (18) above; R² is as defined in any one of paragraphs (19) to (26) above; R³ and R⁴ are as defined in any one of paragraphs (27) to (39) above; R⁵ is as defined in any one of paragraphs (44) to (46) above; R⁶ is as defined in any one of paragraphs (47) to (52) above; R¹⁰ is as defined in any one of paragraphs (73) to (82) above; and n is as defined in any one of paragraphs (92) to (94) above.

In a further group of compounds, the compounds have the structural formula IC shown below:

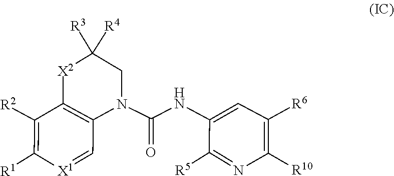

wherein X¹, X², R¹, R², R³, R⁴, R⁵, R⁶, and R¹⁰ are as defined hereinbefore.

In a further group of compounds, the compounds have the structural formula IC shown above, wherein X¹ is as defined in any one of paragraphs (1) to (2); X² is as defined in any one of paragraphs (3) to (6); R¹ is as defined in any one of paragraphs (13) to (18) above; R² is as defined in any one of paragraphs (19) to (26) above; R³ and R⁴ are as defined in any one of paragraphs (27) to (39) above; R⁵ is as defined in any one of paragraphs (44) to (46) above; R⁶ is as defined in any one of paragraphs (47) to (52) above; and R¹⁰ is as defined in any one of paragraphs (73) to (82) above.

In a further group of compounds, the compounds have the structural formula ID shown below:

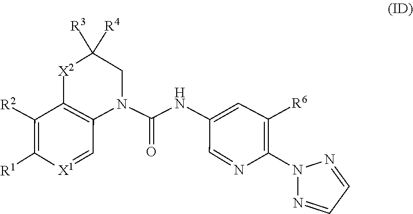

wherein X¹, X², R¹, R², R³, 4, and R⁶ are as defined hereinbefore.

In a further group of compounds, the compounds have the structural formula ID shown above, wherein X¹ is as defined in any one of paragraphs (1) to (2); X² is as defined in any one of paragraphs (3) to (6); R¹ is as defined in any one of paragraphs (13) to (18) above; R² is as defined in any one of paragraphs (19) to (26) above; R³ and R⁴ are as defined in any one of paragraphs (27) to (39) above; and R⁶ is as defined in any one of paragraphs (47) to (52) above.

In a further group of compounds, the compounds have the structural formula IE shown below:

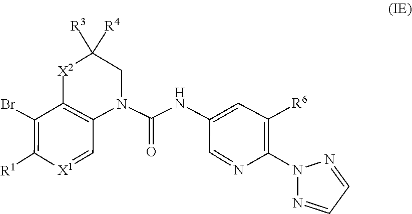

wherein X¹, X², R¹, R³, R⁴, and R⁶ are as defined hereinbefore.

In a further group of compounds, the compounds have the structural formula IE shown above, wherein X¹ is as defined in any one of paragraphs (1) to (2); X² is as defined in any one of paragraphs (3) to (6); R¹ is as defined in any one of paragraphs (13) to (18) above; R³ and R⁴ are as defined in any one of paragraphs (27) to (39) above; and R⁶ is as defined in any one of paragraphs (47) to (52) above.

In a further group of compounds, the compounds have the structural formula IF shown below:

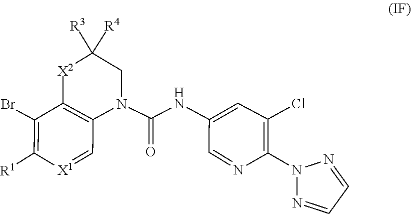

wherein X¹, X², R¹, R³, and R⁴ are as defined hereinbefore.

In a further group of compounds, the compounds have the structural formula IF shown above, wherein X¹ is as defined in any one of paragraphs (1) to (2); X² is as defined in any one of paragraphs (3) to (6); R¹ is as defined in any one of paragraphs (13) to (18) above; and R³ and R⁴ are as defined in any one of paragraphs (27) to (39) above.

In a further group of compounds, the compounds have one of the structural formulae IG, IH or IJ shown below:

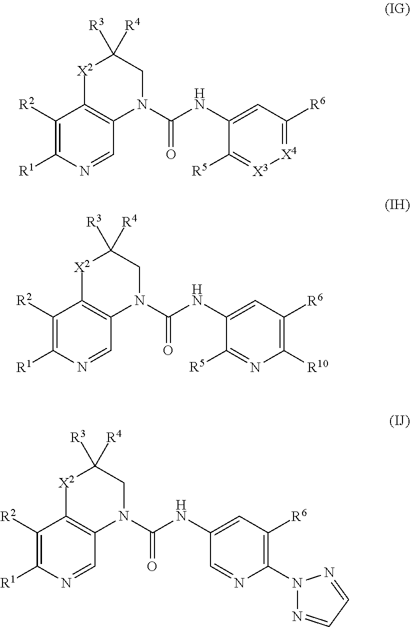

wherein X², X³, X⁴, R¹, R², R³, R⁴, R⁵, R⁶, and R¹⁰ are as defined hereinbefore.

In a further group of compounds, the compounds have one of the structural formulae IG, IH or IJ shown above, wherein X² is as defined in any one of paragraphs (3) to (6); X³ is as defined in any one of paragraphs (7) to (8); X⁴ is as defined in any one of paragraphs (9) to (10); R¹ is as defined in any one of paragraphs (13) to (18) above; R² is as defined in any one of paragraphs (19) to (26) above; R³ and R⁴ are as defined in any one of paragraphs (27) to (39) above; R⁵ is as defined in any one of paragraphs (44) to (46) above; R⁶ is as defined in any one of paragraphs (47) to (52) above; and R¹⁰ is as defined in any one of paragraphs (73) to (82) above.

In an embodiment of a compound of Formula I, or Formulae IA to IJ, when X² is O or NR⁷, then R³ and R⁴ are independently selected from hydrogen, C₁₋₄alkyl, C₂₋₄alkenyl, C₂₋₄alkynyl, C₁₋₄haloalkyl, C₃₋₇cycloalkyl, C₁₋₄haloalkoxy, halo, cyano, 4- to 7-membered heterocyclyl, 5- or 6-membered heteroaryl, and phenyl; wherein said C₁₋₄alkyl, 4- to 7-membered heterocyclyl, 5- or 6-membered heteroaryl, and phenyl are optionally substituted with one or more substituents selected from C₁₋₄alkyl, hydroxy, C₁₋₄alkoxy, halo, and cyano; or R³ and R⁴, together with the carbon atom to which they are attached, form an oxo group, a 4- to 7-membered heterocyclic ring, or a 3- to 6-membered cycloalkyl ring.

Particular compounds of the present invention include any one of the following:
5-Bromo-N-(5-chloro-6-(2H-1,2,3-triazol-2-yl)pyridin-3-yl)-3,4-dihydroquinoline-1 (2H)-carboxamide;
N-(6-(2H-1,2,3-Triazol-2-yl)-5-(trifluoromethyl)pyridin-3-yl)-5-bromo-3,4-dihydroquinoline-1 (2H)-carboxamide;
N-(5-Chloro-6-(2H-1,2,3-triazol-2-yl)pyridin-3-yl)-5-methoxy-3,4-dihydroquinoline-1 (2H)-carboxamide;
N-(6-(2H-1,2,3-Triazol-2-yl)-5-(trifluoromethyl)pyridin-3-yl)-8-bromo-2,3-dihydro-4H-benzo[b][1,4]oxazine-4-carboxamide;
N-(6-(2H-1,2,3-Triazol-2-yl)-5-(trifluoromethyl)pyridin-3-yl)-8-bromo-2-methyl-2,3-dihydro-4H-benzo[b][1,4]oxazine-4-carboxamide;
N-(6-(2H-1,2,3-Triazol-2-yl)-5-(trifluoromethyl)pyridin-3-yl)-8-bromo-2-cyano-2,3-dihydro-4H-benzo[b][1,4]oxazine-4-carboxamide;
N-(5-Chloro-6-(2H-1,2,3-triazol-2-yl)pyridin-3-yl)-5-cyano-3,4-dihydroquinoline-1 (2H)-carboxamide;
N-(6-(2H-1,2,3-Triazol-2-yl)-5-(trifluoromethyl)pyridin-3-yl)-5-(pyridin-3-yl)-3,4-dihydroquinoline-1 (2H)-carboxamide;
N-(6-(2H-1,2,3-Triazol-2-yl)-5-(trifluoromethyl)pyridin-3-yl)-5-(oxazol-2-yl)-3,4-dihydroquinoline-1 (2H)-carboxamide;
N-(6-(2H-1,2,3-Triazol-2-yl)-5-(trifluoromethyl)pyridin-3-yl)-5-(thiazol-2-yl)-3,4-dihydroquinoline-1 (2H)-carboxamide;
Cis-N-(6-(2H-1,2,3-Triazol-2-yl)-5-(trifluoromethyl)pyridin-3-yl)-5-bromo-3,4-dimethyl-3,4-dihydroquinoline-1 (2H)-carboxamide;
N-(6-(2H-1,2,3-Triazol-2-yl)-5-(trifluoromethyl)pyridin-3-yl)-5-phenyl-3,4-dihydroquinoline-1 (2H)-carboxamide;
8-Bromo-N-(5-chloro-6-(2H-1,2,3-triazol-2-yl)pyridin-3-yl)-2,2-dimethyl-2,3-dihydro-4H-benzo[b][1,4]oxazine-4-carboxamide;
N-(6-(2H-1,2,3-triazol-2-yl)-5-(trifluoromethyl)pyridin-3-yl)-5-(1,3,4-thiadiazol-2-yl)-3,4-dihydroquinoline-1 (2H)-carboxamide;
5-Bromo-N-(5-chloro-6-(2H-1,2,3-triazol-2-yl)pyridin-3-yl)-3,3-dimethyl-3,4-dihydroquinoline-1 (2H)-carboxamide;
5-Bromo-N-(5-chloro-6-(2H-1,2,3-triazol-2-yl)pyridin-3-yl)-4-methyl-3,4-dihydroquinoxaline-1 (2H)-carboxamide;
5-Bromo-N-(5-chloro-6-(2H-1,2,3-triazol-2-yl)pyridin-3-yl)-3,4-dihydro-1,7-naphthyridine-1 (2H)-carboxamide;
8-Bromo-N-(5-chloro-6-(2H-1,2,3-triazol-2-yl)pyridin-3-yl)-2-cyano-2-methyl-2,3-dihydro-4H-benzo[b][1,4]oxazine-4-carboxamide;
8-Bromo-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-ethynyl-2,3-dihydro-4H-benzo[b][1,4]oxazine-4-carboxamide;
8-Bromo-N-(5-chloro-6-(2H-1,2,3-triazol-2-yl)pyridin-3-yl)-2,3-dihydro-4H-pyrido[4,3-b][1,4]oxazine-4-carboxamide;
N-(5-Chloro-6-(2H-1,2,3-triazol-2-yl)pyridin-3-yl)-5-cyclopropyl-3,4-dihydro-1,7-naphthyridine-1 (2H)-carboxamide;
N-(6-(2H-1,2,3-Triazol-2-yl)-5-(trifluoromethyl)pyridin-3-yl)-8-bromo-2,2-dimethyl-2,3-dihydro-4H-benzo[b][1,4]oxazine-4-carboxamide;
N-(5-Chloro-6-(2H-1,2,3-triazol-2-yl)pyridin-3-yl)-8-cyclopropyl-2,2-dimethyl-2,3-dihydro-4H-pyrido[4,3-b][1,4]oxazine-4-carboxamide;
(S)-1-Bromo-N-(5-chloro-6-(2H-1,2,3-triazol-2-yl)pyridin-3-yl)-6a,7,8,9-tetrahydropyrido[3,4-e] pyrrolo[1,2-a]pyrazine-5 (6H)-carboxamide;
(R)-8-Bromo-N-(5-chloro-6-(2H-1,2,3-triazol-2-yl)pyridin-3-yl)-1,2-dimethyl-2,3-dihydropyrido[3,4-b]pyrazine-4 (1H)-carboxamide;
8′-Bromo-N-(5-chloro-6-(2H-1,2,3-triazol-2-yl)pyridin-3-yl)-4,5-dihydro-2H-spiro [furan-3,2′-pyrido[4,3-b][1,4]oxazine]-4′ (3′H)-carboxamide;
1-Bromo-N-(5-chloro-6-(2H-1,2,3-triazol-2-yl)pyridin-3-yl)-6,6a,7,8,9,10-hexahydro-5H-dipyrido[1,2-a: 3′,4′-e] pyrazine-5-carboxamide;
8-Bromo-N-(5-chloro-6-(2H-1,2,3-triazol-2-yl)pyridin-3-yl)-1-methyl-2,3-dihydropyrido[3,4-b] pyrazine-4 (1H)-carboxamide;
8-Bromo-N-(5-chloro-6-(2H-1,2,3-triazol-2-yl)pyridin-3-yl)-2-methyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-1,2,2-trimethyl-2,3-dihydropyrido[3,4-b]pyrazine-4 (1H)-carboxamide;
8-Chloro-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-1-(tetrahydro-2H-pyran-4-yl)-2,3-dihydropyrido[3,4-b]pyrazine-4 (1H)-carboxamide;
(R)-8-Bromo-N-(5-chloro-6-(2H-1,2,3-triazol-2-yl)pyridin-3-yl)-2-ethyl-1-methyl-2,3-dihydropyrido[3,4-b]pyrazine-4 (1H)-carboxamide;
(R)-8-Bromo-N-(5-chloro-6-(2H-1,2,3-triazol-2-yl)pyridin-3-yl)-2-isopropyl-1-methyl-2,3-dihydropyrido[3,4-b]pyrazine-4 (1H)-carboxamide;
7-Amino-8-bromo-N-(5-chloro-6-(2H-1,2,3-triazol-2-yl)pyridin-3-yl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydropyrido[3,4-b]pyrazine-4 (1H)-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-7-methyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-ethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-isopropyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-cyclopropyl-2,3-dihydro-4H-pyrido[4,3-b][1,4]oxazine-4-carboxamide;
N-(5-Chloro-6-(2H-1,2,3-triazol-2-yl)pyridin-3-yl)-8-cyano-7-methyl-2-(trifluoromethyl)-2,3-dihydro-4H-pyrido[4,3-b][1,4]oxazine-4-carboxamide;
N-(5-Chloro-6-(2H-1,2,3-triazol-2-yl)pyridin-3-yl)-8-cyano-2-(trifluoromethyl)-2,3-dihydro-4H-pyrido[4,3-b][1,4]oxazine-4-carboxamide;
N-(5-Chloro-6-(2H-1,2,3-triazol-2-yl)pyridin-3-yl)-8-methoxy-2-(trifluoromethyl)-2,3-dihydro-4H-pyrido[4,3-b][1,4]oxazine-4-carboxamide;
5-Bromo-N-(5-chloro-6-(2H-1,2,3-triazol-2-yl)pyridin-3-yl)-3-cyano-3,4-dihydro-1,7-naphthyridine-1 (2H)-carboxamide;
N-(6-(2H-1,2,3-triazol-2-yl)-5-(trifluoromethyl)pyridin-3-yl)-8-cyano-7-methyl-2-(trifluoromethyl)-2,3-dihydro-4H-pyrido[4,3-b][1,4]oxazine-4-carboxamide;
8-cyano-N-(5-cyano-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide;
8-cyano-N-(6-(cyclopropyl(methyl) carbamoyl)-5-(difluoromethyl)pyridin-3-yl)-7-methyl-2-(trifluoromethyl)-2,3-dihydro-4H-pyrido[4,3-b][1,4]oxazine-4-carboxamide;
8-cyano-7-methyl-2-(trifluoromethyl)-N-(2-(trifluoromethyl)pyridin-4-yl)-2,3-dihydro-4H-pyrido[4,3-b][1,4]oxazine-4-carboxamide; and
N-(5-chloro-6-(difluoromethoxy)pyridin-3-yl)-8-cyano-7-methyl-2-(trifluoromethyl)-2,3-dihydro-4H-pyrido[4,3-b][1,4]oxazine-4-carboxamide;
   or a pharmaceutically acceptable salt thereof.

The various functional groups and substituents making up the compounds of the present invention are typically chosen such that the molecular weight of the compound does not exceed 1000. More usually, the molecular weight of the compound will be less than 750, for example less than 700, or less than 650.

Suitable or preferred features of any compounds of the present invention may also be suitable features of any other aspect.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention typically possess one or more asymmetric centers; such compounds can therefore be produced as individual (R)- or(S)-stereoisomers or as mixtures thereof. Unless indicated otherwise, the description or naming of a particular compound in the specification and claims is intended to include both individual enantiomers, diastereoisomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess MALT1 inhibitory activity.

The present invention also encompasses compounds of the invention as defined herein which comprise one or more isotopic substitutions. For example, H may be in any isotopic form, including ¹H, ²H (D) and ³H (T); C may be in any isotopic form including ¹²C, ¹³C, and ¹⁴C; and O may be in any isotopic form, including ¹⁶O and ¹⁸O; and the like.

It is also to be understood that certain compounds of the invention may exist in solvated as well as unsolvated forms such as, for example, hydrated forms. It is to be understood that the invention encompasses all such solvated forms that possess MALT1 inhibitory activity.

It is also to be understood that certain compounds of the invention may exhibit polymorphism, and that the invention encompasses all such forms that possess MALT1 inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

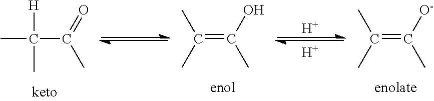

Compounds of the invention containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

It shall also be appreciated that compounds of the formula I may also be covalently linked (at any suitable position) to other groups such as, for example, solubilising moieties (for example, PEG polymers), moieties that enable them to be bound to a solid support (such as, for example, biotin-containing moieties), and targeting ligands (such as antibodies or antibody fragments).

### Synthesis

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, “Protecting groups in Organic Synthesis (3rd Ed), John Wiley & Sons, NY (1999)”, T. Greene & P. Wuts. Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable protecting group for an amino or alkylamino group is, for example, a substituted benzyl group such as 4-methoxybenzyl or 2,4-dimethoxybenzyl. Such a protecting group may be removed by, for example, by treatment with by treatment with a suitable acid as hydrochloric, sulfuric or phosphoric acid or trifluoroacetic acid. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the invention may be prepared, in known manner, in a variety of ways. Compounds of Formula I can be prepared by the methods given below, by the methods given in the General Procedures or Synthesis of Examples or by analogous methods. The routes described are merely illustrative of some of the methods that can be employed for the synthesis of compounds of Formula I and the person skilled in the art will appreciate that the order of the reaction steps is not limited to those described. It will also be appreciated that the assignment of nucleophile and electrophile is not limited to that described herein and in some cases it may be appropriate for the assignment to be reversed. Different approaches to synthetic chemistry strategy are described in “Organic Synthesis: The Disconnection Approach”, 2nd edition, S. Warren and P. Wyatt (2008).

Compounds of Formula I, wherein R¹, R², R³, R⁴, R⁵, Re, X¹, X², X³ and X⁴ are as previously defined, can be prepared by the condensation of compounds of Formula II and Formula III in step (a) using a suitable coupling reagent (Scheme A). A suitable coupling reagent includes, for example, phosgene, diphosgene, triphosgene, phenylchloroformate, 4-nitrophenylchloroformate or 1,1-carbonyldimidazole.

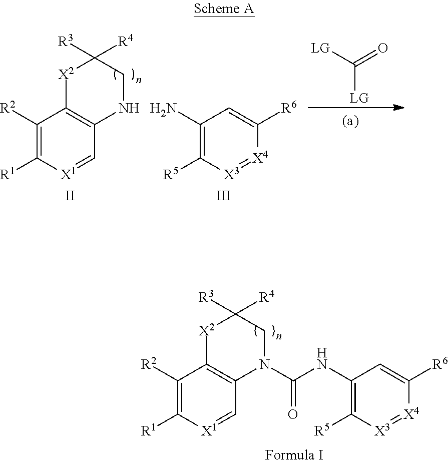

Compounds of Formula I, wherein R¹, R², R³, R⁴, R⁵, R⁶, X¹, X², X³ and X⁴ are as previously defined, may also be prepared by conversion of an acid of Formula IV to the corresponding isocyanate, in step (b), by use of, for example, diphenylphosphoryl azide, and coupling with an amine of Formula II as shown in Scheme B.

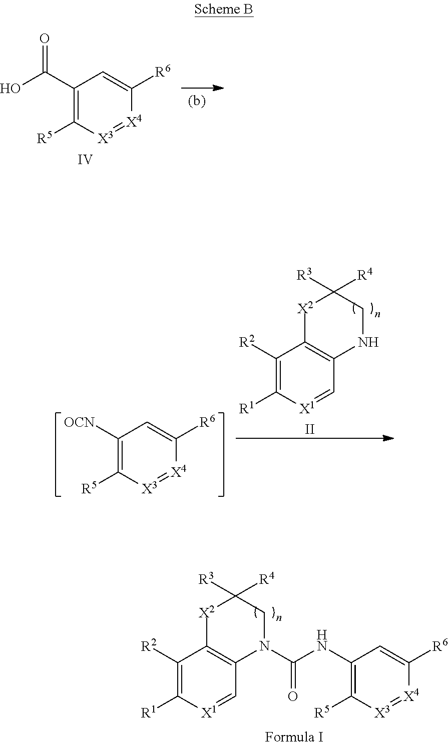

The general preparation of some typical compounds of Formula II are described below and are generally prepared from starting materials which are either commercially available or prepared by standard synthetic processes commonly used by those skilled in the art of organic chemistry. Compounds of Formula III and IV may be prepared using analogous reaction protocols as described in WO2015181747, WO2017081641, WO2018020474 and WO202008222.

A compound of Formula II, wherein R¹, R², R³, R⁴ and X¹ are as previously defined and X²=O, may be formed as shown in Scheme C.

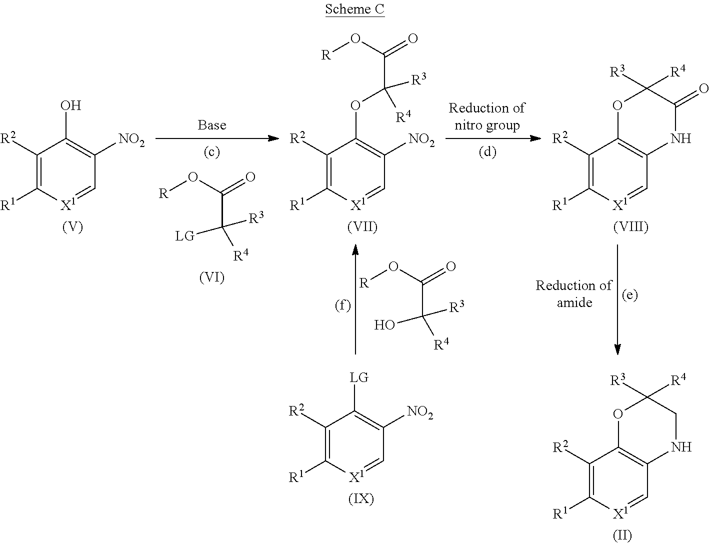

Base promoted alkylation of a suitable phenol of Formula V with a suitable alkylating agent of Formula VI containing a leaving group (LG) step (c) furnishes a compound of Formula VII. Suitable bases include, for example, sodium hydride, sodium bis(trimethylsilyl)amide, lithium diisopropylamide or triethylamine and leaving groups include, for example, chlorine, bromine or iodine. An alternative route to a compound of Formula VII consists of the displacement of a suitable aromatic leaving group step (f) in a compound of Formula IX with the appropriate alcohol. Suitable leaving groups include, for example, fluorine or chlorine. Reduction of the nitro group in a compound of Formula VII with an appropriate reducing agent coupled with subsequent cyclisation affords a cyclic amide compound of Formula VIII step (d). Appropriate reducing agents include, for example, zinc in acetic acid, iron in acetic acid or palladium on charcoal in the presence of hydrogen. Reduction of the compound of Formula VIII with a suitable reducing group step (e) furnishes compound of Formula II. Suitable reducing agents include, for example, BH₃—Me₂S, lithium aluminium hydride or BH₃-THF.

A compound of Formula II, wherein R¹, R², R³, R⁴, R⁷ and X¹ are as previously defined and X²=N7, may be formed as shown in Scheme D. Displacement of a suitable leaving group step (g) in a compound of Formula IX by an appropriate aminoester of Formula X affords a compound of Formula XI. Suitable leaving groups include, for example, fluorine or chlorine. Steps (d) and (e) are described for Scheme C.

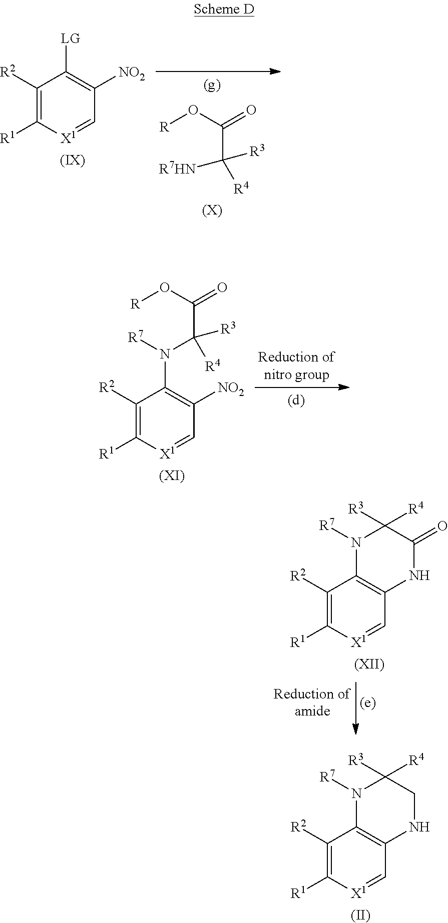

A compound of Formula II, wherein R¹, R², R⁷ and X¹ are as previously defined and X²=NR⁷, may be formed as shown in Scheme E. Displacement of a suitable leaving group by a primary amine of Formula XIII step (h) affords a compound of Formula XIV. Suitable leaving groups include, for example, fluoride or chloride. Step (d) is as described for Scheme C. The ring closure to afford a compound of Formula II can be achieved using a suitable “ethylene synthon”. Suitable ethylene synthons include, for example, glyoxal in the presence of a reducing agent such as NaBH(OAc)₃ or dibromoethane in the presence of a base such as triethylamine.

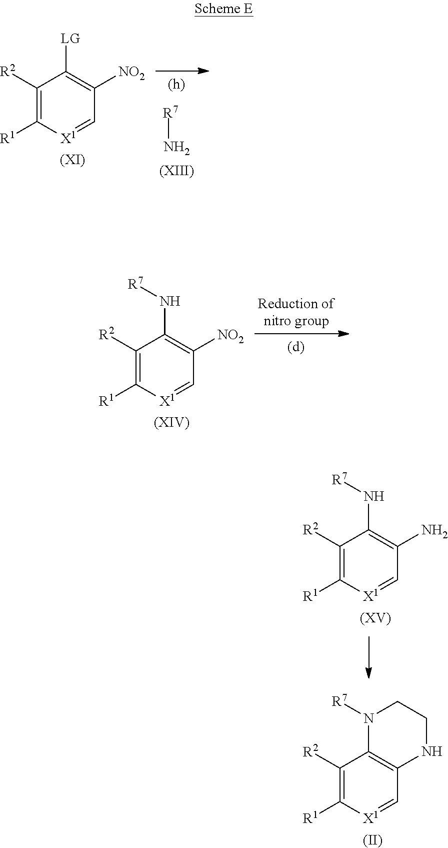

A compound of Formula II, wherein R¹, R², R³, R⁴, X¹ and X² are as previously defined, may be formed as shown in Scheme F.

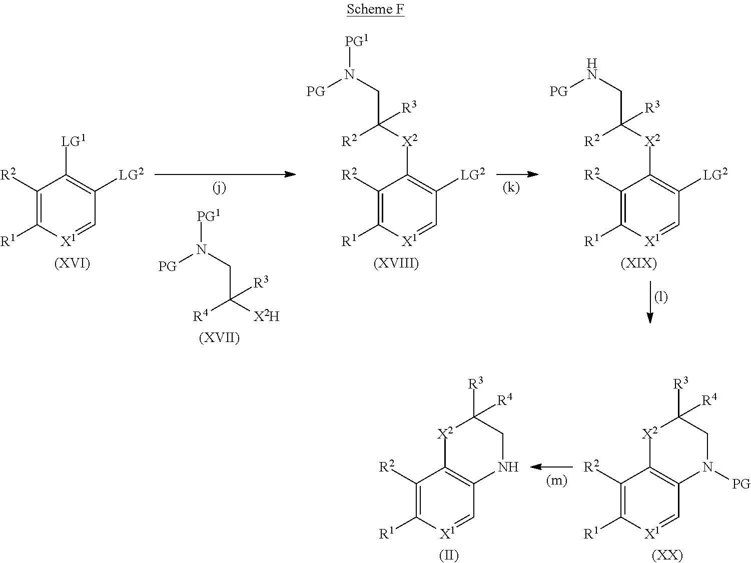

Selective displacement of leaving group LG¹ in a compound of Formula XVI by a suitably protected diamine (wherein X² is NR⁷) or ethanolamine (wherein X² is O) of Formula XVII affords a compound of Formula XVIII step (j). Suitable LG¹ leaving groups include, for example, fluoride or chloride. Selective removal of protecting group PG¹ step (k) affords a compound of Formula XIX. Suitable PG¹ include, for example, tert-butoxycarbonyl which may be removed with acid at room temperature. Transition metal catalysed displacement of leaving group LG² by the nitrogen atom affords a compound of Formula XX step (I). Suitable transition metal catalysed cyclisations include, for example, the use of Pd₂(dba)₃ in the presence of BINAP and an appropriate base such as t-BuONa reacting with a suitable leaving group LG² such as bromine or iodine. Removal of the final protecting group PG step (m) affords a compound of Formula II. Suitable PG include, for example, 2,4-dimethoxybenzyl which may be removed with acid at elevated temperature.

A compound of Formula II, wherein R¹, R², R³, R⁴, R⁸, R⁹ and X¹ are as previously defined and X²=CR⁸R⁹, may be formed as shown in Scheme G. Conversion of a compound of Formula XXI to a suitable organometallic reagent and subsequent addition to a compound of Formula XXII step (n) affords a compound of Formula XXIII. Conversion to a suitable organometallic reagent can be achieved, for example, by reaction with palladium tetrakistriphenylphospine and a suitable base such as sodium carbonate. Steps (d) and (e) are described for Scheme C. A further substituent, such as R⁴, can be incorporated by treating a compound of Formula XXIV with a suitable base and R⁴-LG step (o). Suitable bases include, for example, sodium hydride, sodium bis(trimethylsilyl)amide and lithium diisopropylamide and a suitable LG includes, for example bromine, iodide or triflate.

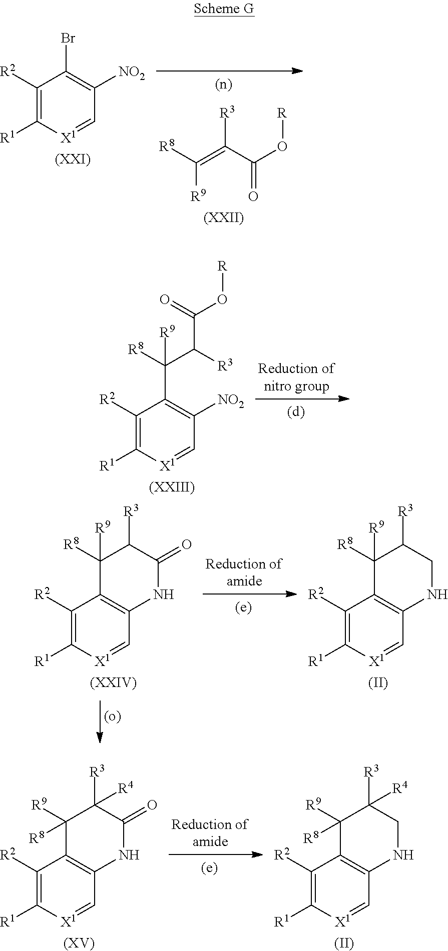

A compound of Formula II, wherein R¹, R², R³, R⁴, X¹ and X² are as previously defined, may be formed as shown in Scheme H. Alkylation step (p) of a compound of Formula XXVI, prepared as described in Schemes C and D where R³ and R⁴ are H, by use of an appropriate base and R³LG and R⁴LG results in the formation of a compound of Formula XXVII-either sequentially (R³≠R⁴) or potentially in a single step (R³=R⁴). Suitable bases include, for example, sodium hydride, sodium bis(trimethylsilyl)amide and lithium diisopropylamide and a suitable LG includes, for example bromine, iodide or triflate. Step (e) as described for Scheme C.

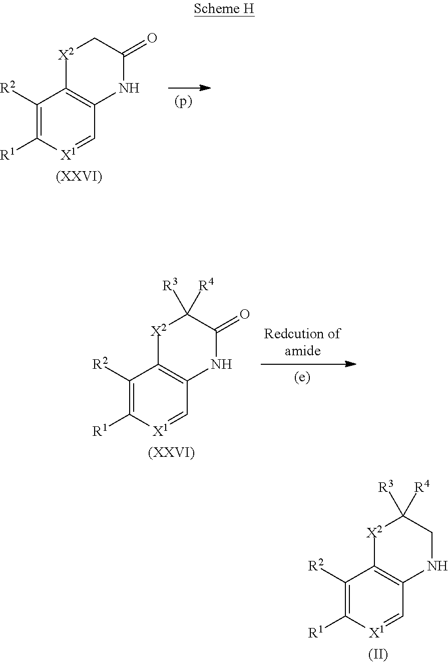

A compound of Formula II, wherein R¹, R², R³, R⁴, X¹ and X² are as previously defined, may be formed as shown in Scheme I. The bromo compound of Formula XXVIII can be converted to a range of R² groups. This conversion includes, for example, conversion of the bromo to the corresponding boronate acid/ester and subsequent reaction with a suitable R²—Br or reaction with a suitable R²—B(OH)₂ (or boronate ester). Step (e) as described for Scheme C.

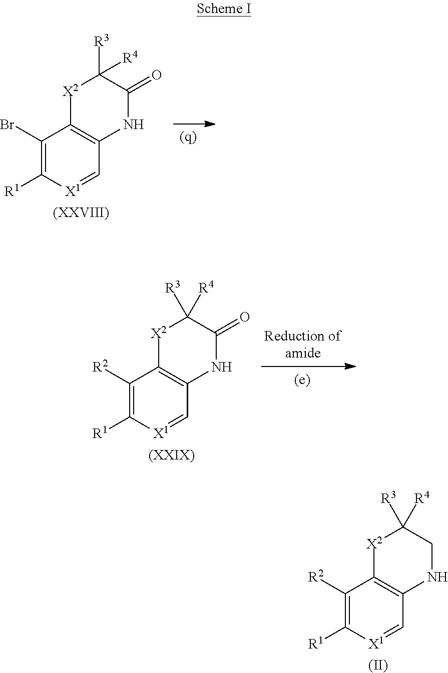

A compound of Formula II, wherein R¹, R², R³, R⁴ and X¹ are as previously defined and X² is absent, may be formed as shown in Scheme J. Alkylation step (q) of a compound of Formula XXX by use of an appropriate base and R³LG and R⁴LG results in the formation of a compound of Formula XXXI-either sequentially (R³≠R⁴) or potentially in a single step (R³=R⁴). Suitable bases include, for example, sodium hydride, sodium bis(trimethylsilyl)amide and lithium diisopropylamide and a suitable LG includes, for example bromine, iodide or triflate. Step (e) as described for Scheme C.

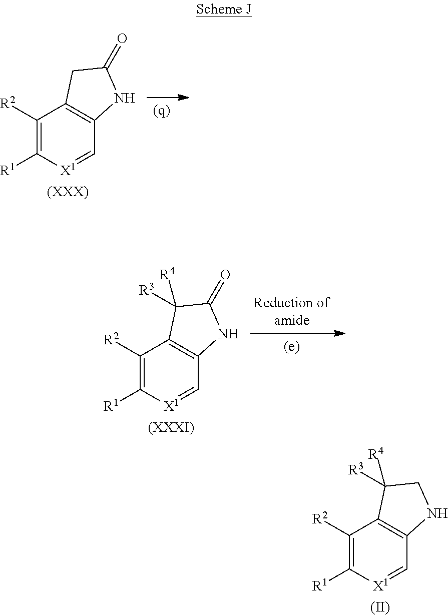

A compound of Formula II, wherein R¹, R², R⁸, R⁹, R³, R⁴, and X¹ are as previously defined and X² is CR⁸ or CR⁹, may be formed as shown in Scheme K. Reduction of the pyridyl ring step (r) of a compound of Formula XXXII furnishes a compound of Formula II.

Suitable reduction conditions include, for example, hydrogen in the presence of platinum (IV) oxide or hydrogen in the presence of IrCl₂(COD).

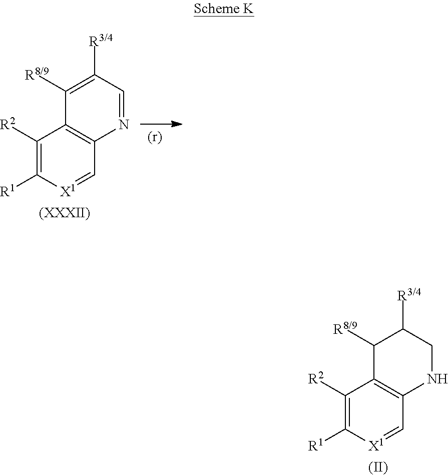

A compound of Formula II, wherein R¹, R², R⁸, R⁹, R³, R⁴, and X¹ are as previously defined, may be formed as shown in Scheme L. Ring expansion step(s) of a ketone of Formula XXXIII can form a compound of Formula XXV. Suitable reduction conditions include, for example, the use of sodium azide in the presence of an acid, for example, sulfuric acid. Step (e) as described for Scheme C.

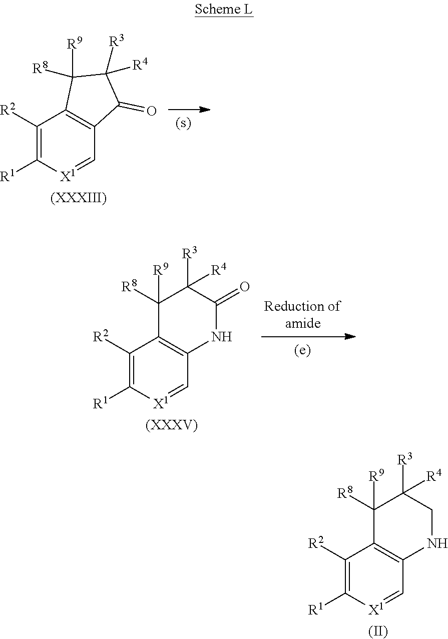

A compound of Formula II, wherein R¹, R², R⁸, R⁹, R³, R⁴, and X¹ are as previously defined and X² is CR⁸R⁹, may be formed as shown in Scheme M. Annulation step (t) of a suitable acrylamide of Formula XXXIV may afford the amide of Formula XXXV. Suitable annulation conditions, include, for example the use of aluminium trichloride. Step (e) as described for Scheme C.

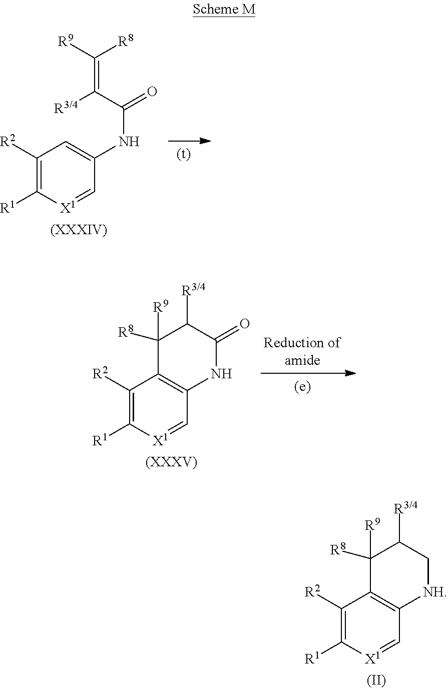

### Pharmaceutical Compositions

The compounds of the invention will normally, but not necessarily, be formulated into pharmaceutical compositions prior to administration to a patient. Therefore, according to a further aspect of the invention there is provided a pharmaceutical composition which comprises a compound of the invention as defined hereinbefore, or a pharmaceutically acceptable salt thereof, and one or more pharmaceutically acceptable excipients, diluent or carrier.

The pharmaceutical compositions of the invention may be prepared and packaged in bulk form wherein a safe and effective amount of a compound of the invention can be extracted and then given to the patient such as with powders or syrups.

Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The compositions of the invention may be in a form suitable for oral use (for example as tablets, capsules, caplets, pills, troches, powders, syrups, elixirs, suspensions, solutions, emulsions, sachets, and cachets), for topical use (for example as creams, ointments, lotions, solutions, pastes, sprays, foams, and gels), for transdermal administration such as via transdermal patches, for administration by inhalation (for example as a dry powders, aerosols, suspensions, and solutions), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must be of sufficiently high purity to render it pharmaceutically-acceptable.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The person skilled in the art will appreciate that certain pharmaceutically-acceptable excipients may serve more than one function and may serve alternative functions depending on how much of the excipient is present in the formulation and what other ingredients are present in the formulation.

Persons skilled in the art possess the knowledge and skill to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Routes of Administration

The compounds of the invention or pharmaceutical composition comprising the active compound may be administered to a subject by any convenient route of administration, whether systemically/peripherally or topically (i.e. at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In a preferred embodiment, a compound of the invention as defined herein, or a pharmaceutically acceptable salt thereof, or a pharmaceutical composition as defined herein, is administered orally or via injection, such as conveniently by oral administration.

### Therapeutic Uses and Applications

The compounds of the invention are inhibitors of MALT1. As a consequence, they are potentially useful therapeutic agents for the treatment of diseases or conditions mediated by MALT1.

Thus, in one aspect, the present invention relates to a compound of the invention as defined herein, or a pharmaceutically acceptable salt thereof, or a pharmaceutical composition as defined herein, for use in therapy.

In another aspect, the present invention relates to a compound of the invention as defined herein, or a pharmaceutically acceptable salt thereof, or a pharmaceutical composition as defined herein, for use in the treatment of diseases or disorders mediated by MALT1.

In another aspect, the present invention relates to a method of treating a disease or disorders mediated by MALT1, said method comprising administering to a subject in need of such treatment a therapeutically effective amount of a compound of the invention as defined herein, or a pharmaceutically acceptable salt thereof, or a pharmaceutical composition as defined herein.

Examples of particular diseases or conditions that the compounds of formula (I) and their pharmaceutically acceptable salts may be used to treat include, but are not limited to:
i) lymphomas, leukaemias, carcinomas, and sarcomas; such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, or GIST (gastrointestinal stromal tumour); and
ii) immunological diseases including autoimmune and inflammatory disorders; such as arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

In particular, the compounds of the invention (including pharmaceutically acceptable salts) may be used in the treatment of lymphomas, such as non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

In another aspect, the present invention provides a compound, or a pharmaceutically acceptable salt thereof, or a pharmaceutical composition as defined herein, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, said method comprising administering to a subject in need of such treatment a therapeutically effective amount of a compound, or a pharmaceutically acceptable salt thereof, or a pharmaceutical composition as defined herein.

In another aspect, the present invention provides a method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL), said method comprising administering to a subject in need of such treatment a therapeutically effective amount of a compound, or a pharmaceutically acceptable salt thereof, or a pharmaceutical composition as defined herein.

In another aspect, the present invention provides a method of inhibiting MALT1 in vitro, said method comprising administering an effective amount of a compound, or a pharmaceutically acceptable salt thereof.

In another aspect, the present invention provides a method of inhibiting MALT1 in vivo, said method comprising administering an effective amount of a compound, or a pharmaceutically acceptable salt thereof.

In another aspect, the present invention provides a method of inhibiting MALT1 in vitro and/or in vivo, said method comprising contacting a cell with an effective amount of a compound as defined herein, or a pharmaceutically acceptable salt thereof.

### Combination Therapies

The compounds of the invention may be administered alone as a monotherapy or may administered in combination with one or more additional therapeutic agents. The selection of the one or more additional therapeutic agents will of course vary depending on the disease or condition to be treated and its severity.

It is commonplace to use combination therapies to treat certain medical conditions.

According to a particular aspect of the invention there is provided a combination suitable for use in the treatment of a disease or condition in which MALT1 is implicated, comprising a compound of the invention as defined hereinbefore, or a pharmaceutically acceptable salt thereof, and another therapeutic agent.

According to this aspect of the invention there is provided a combination suitable for use in the prevention or treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenström macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, the combination comprising a compound of the invention as defined hereinbefore, or a pharmaceutically acceptable salt thereof, and one or more additional therapeutic agents.

In a further aspect of the invention there is provided a compound of the invention or a pharmaceutically acceptable salt thereof, in combination with one or more additional therapeutic agents.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt thereof in combination with one or more additional therapeutic agents in association with a pharmaceutically acceptable diluent or carrier.

The one or more additional therapeutic agents may comprise a further compound of the present invention. Therefore, in an embodiment, there is provided a pharmaceutical composition which comprises two compounds of the invention, or pharmaceutically acceptable salts thereof, in association with a pharmaceutically acceptable diluent or carrier.

According to a particular aspect of the invention there is provided a combination suitable for use in the prevention or treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, chronic lymphocytic leukaemia (CLL), or small lymphocytic lymphoma (SLL).

Examples of other therapeutic agents that may be used as part of a combination therapy with a compound of the present invention (e.g. as one of two or more active agents as part of double or triple combinations) include, but are not limited to, the following:
(i) BTK (Bruton's tyrosine kinase) inhibitors such as ibrutinib, acalabrutinib, zanubrutinib, tirabrutinib, orelabrutinib, evobrutinib, fenebrutinib, rilzabrutinib, tolebrutinib, MK1026 (ARQ-531), LOXO-305, elsubrutinib, poseltinib, branebrutinib, spebrutinib, luxeptinib, DTRM-555, JnJ64264681, BGB-3959, AS-1763 and remibrutinib;
(ii) SYK inhibitors such as fostamatinib, entospletinib, HMPL-523, IC-265, SKI-O-703, cerdulatinib, PRT-2761, GSK-264264, SYHX-1901, MK-8457, HM-43239, R-348 and PUR-1800;
(iii) PKC inhibitors such as darovasertib, MS-553, enzastaurin, safingol, ruboxistaurin and AR-13503;
(iv) PI3K pathway inhibitors such as alpelisib, duvelisib, copanlisib, idelalisib, umbralisib, serabelisib, CHF-6523, BDP-681, zandelisib, ART-001, buparlisib, OP-11, HMPL-689, dezapelisib, seletalisib, epivotide, IOA-244, SHC-014748M, LX-086, inavolisib, MEN-1611, eganelisib, leniolisib, ACP-319, BGB-10188, CYH-33, HS-10352, CMX-2043, ZX-101A, KA-2237, VS-5584, ASN-003, TQ-B325, AL-58805, gedatolisib, HEC-68498, CLL-442, tenalisib, dactolisib, GSK-2126458 and bimiralisib;
(v) BCL family inhibitors such as BCL-201, AZD-0466, navitoclax, pelcitoclax, LP-118, FCN-338, BGB-11417, UBX-1325, LP-108, VOB-560, lisaftoclax, venetoclax, ZN-d5 and ABBV-167;
(vi) JAK inhibitors such as gusacitinib, delgocitinib, tofacitinib, abrocitinib, ruxolitinib, baricitinib, fedratinib, upadacitinib, filgotinib, peficitinib, TD-8236, TD-0903, CEE-321, lorpucitinib, WXSH-0150, SYHX-1901, cerdulatinib, izencitinib, KL-130008, WP-1066, gusacitinib, INCB-52793, AC-1101, ATI-1777, SHR-0302, CPL-409116, momelotinib, brepocitinib, TTL-018, TD-5202, LP-0184, INCB-054707, jaktinib, TQ-05105, itacitinib, AZD-0449, GLPG-0555, LW-104, ARQ-252, WXFL-10203614, golidocitinib, deuruxolitinib, CJ-15314, CS-12192, ritlecitinib, R-348, ATI-2138 and ilginatinib;
(vii) PIM kinase inhibitors such as uzansertib, TP-3654, MEN-1703, ETH-155008, abemaciclib and SF-1126;
(viii) mTORC inhibitors such as rapamycin, sirolimus, novolimus, umirolimus zotarolimus, temsirolimus, everolimus, merilimus, eRapa, ridaforolimus;
(ix) Rituximab or other B cell antigen-binding antibodies as well as immune cell redirection agents (e.g. blinatumomab or CAR-T cells);
(x) Anti-PD1 antibodies such as nivolumab, pembrolizumab, lambrolizumab, pidilzumab, BGB-A317;
(xi) Anti-PD-L1 antibodies such as atezolizemab, avelumab, durvalumab, MEDI-4736 and MPDL3280A;
(xii) Antibodies that inhibit the 4-1BB-ligand interaction such as utomilumab; and
(xiii) Antibodies that inhibit the interaction of CTLA-4 and its ligands such as ipilumumab, tremelimumab, or those disclosed in WO2014/207063.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

Such conjoint/combination treatment may be achieved by way of the simultaneous, sequential or separate dosing of the individual components of the treatment. In one embodiment, the individual compounds will be administered simultaneously in a combined pharmaceutical formulation.

Such combination therapies employ the compounds of this invention within the dosage range described herein and the other pharmaceutically active agent within approved dosage ranges and/or the dosage such as described in the relevant publication reference.

### Examples

### General Procedures:

Methods for preparing the compounds of this invention are illustrated in the following Examples. Starting materials are made according to procedures known in the art or as illustrated herein or are available commercially. Commercial reagents were used without further purification. Where no reaction temperature is included, the reaction was performed at ambient temperature which is typically 17-27° C.

A person skilled in the art will appreciate that reaction temperatures, reaction times & reagent quantities may be varied from those stated herein.

Where compounds described in the invention are characterized by 1H NMR spectroscopy, spectra were recorded on 400 MHZ Bruker instrument. Where no temperature is included, the spectra were recorded at ambient temperature. Chemical shift values are expressed in parts per million (ppm). The following abbreviations are used for the multiplicity of the NMR signals: s=singlet, br=broad, t=triplet, q=quartet, m=multiplet, d=doublet.

Where compounds described in the invention are characterized by LCMS data, retention time and molecular weight are determined using the conditions listed below.
Method 1: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% formic acid in water [eluent A], 0.1% formic acid in MeCN [eluent B] (Flow 0.6 mL/min). Gradient: 5-95% B in 2.2 min, hold at 95% for 1.2 min; column temp 40° C.
Method 2: Agilent 1260 (Waters Acquity PDA 210-400 nm and Waters Acquity SQ detector). Column: Luna Omega C18, 100 Å, 1.6 μm, 50×2.1 mm. Conditions: 0.05% formic acid in water [eluent A], 0.05% formic acid in MeCN [eluent B] (Flow 0.6 mL/min). Gradient: 5-95% B in 2.2 min, hold at 95% for 1.2 min; column temp 40° C.
Method 3: Agilent 1260 (Waters Acquity PDA 210-400 nm and Waters Acquity SQ detector). Column: Waters Acquity BEH C18, 130 Å, 1.7 μm, 50×2.1 mm. Conditions: 0.05% formic acid in water [eluent A], 0.05% formic acid in MeCN [eluent B] (Flow 0.6 mL/min). Gradient: 5-95% B in 6.7 min, hold at 95% for 1.2 min; column temp 40° C.
Method 4: Agilent 1260 (Waters Acquity PDA 210-400 nm and Waters Acquity SQ detector). Column: Waters Acquity BEH C18, 130 Å, 1.7 μm, 50×2.1 mm. Conditions: 0.05% formic acid in water [eluent A], 0.05% formic acid in MeCN [eluent B] (Flow 0.6 mL/min). Gradient: 5-95% B in 2.2 min, hold at 95% for 1.2 min; column temp 40° C.
Method 5: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% formic acid in water [eluent A], 0.1% formic acid in MeCN [eluent B] (Flow 0.6 mL/min). Gradient: 5-95% B in 1.7 min, hold at 95% for 1.5 min; column temp 40° C.
Method 6: Dionex Ultima 3000 (Avalon PDA 210-400 nm and Thermo Scientific LCQ lon Trap detector). Column: Luna Omega PS C18, 100 Å, 3 μm, 50×2.1 mm. Conditions: 95:5 10 mM ammonium formate in water/MeCN [eluent A], 5:95 10 mM ammonium formate in water/MeCN [eluent B] (Flow 1.0 mL/min). Gradient: 2-98% B in 3 min, hold at 98% for 0.5 min; column temp 40° C.
Method 7: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B] (Flow 0.6 mL/min). Gradient: 2-98% B in 2.9 min, hold at 98% for 1.2 min; column temp 40° C.
Method 8: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B] (Flow 0.5 mL/min). Gradient: 2-98% B in 2.9 min, hold at 98% for 1.2 min; column temp 45° C.
Method 9: Dionex Ultima 3000 (Avalon PDA 210-400 nm and Thermo Scientific LCQ lon Trap detector). Column: Luna Omega PS C18, 100 Å, 3 μm, 50×2.1 mm. Conditions: 95:5 10 mM ammonium formate in water/MeCN [eluent A], 5:95 10 mM ammonium formate in water/MeCN [eluent B] (Flow 1.0 mL/min). Gradient: 2-98% B in 1.9 min, hold at 98% for 2.2 min; column temp 40° C.
Method 10: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B] (Flow 0.6 mL/min). Gradient: 2-98% B in 2.9 min, hold at 98% for 1.2 min; column temp 45° C.
Method 11: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM NH₄HCO₃ in water [eluent A], MeCN [eluent B] (Flow 0.5 mL/min). Gradient: 3-100% B in 6.7 min, hold at 100% for 1.2 min; column temp 40° C.
Method 12: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM NH₄HCO₃ in water [eluent A], MeCN [eluent B] (Flow 0.5 mL/min). Gradient: 5-95% B in 2.2 min, hold at 95% for 1.2 min; column temp 40° C.
Method 13: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B] (Flow 0.5 mL/min). Gradient: 5-95% B in 2.9 min, hold at 95% for 1.2 min; column temp 40° C.
Method 14: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% formic acid in water [eluent A], MeCN [eluent B] (Flow 0.6 mL/min). Gradient: 5-95% B in 2.2 min, hold at 95% for 1.2 min; column temp 40° C.
Method 15: Agilent 1260 (Waters Acquity PDA 210-400 nm and Waters Acquity SQ detector). Column: Waters Acquity BEH C18, 130 Å, 1.7 μm, 50×2.1 mm. Conditions: 10 mM ammonium acetate in water [eluent A], MeCN [eluent B] (Flow 0.6 mL/min). Gradient: 2-65% B in 7.7 min, hold at 65% for 1 min, 65-90% over 1 min, hold at 90% 1 min; column temp 40° C.
Method 16: Agilent 1260 (Waters Acquity PDA 210-400 nm and Waters Acquity SQ detector). Column: Waters Acquity BEH C18, 130 Å, 2.5 μm, 50×3.5 mm. Conditions: 5 mM NH₄HCO₃ in water [eluent A], MeCN [eluent B] (Flow 1.0 mL/min). Gradient: 5-95% B in 1.7 min, hold at 95% for 2 min; column temp 40° C.
Method 17: Agilent 1260 (Waters Acquity PDA 210-400 nm and Waters Acquity SQ detector). Column: Waters Acquity BEH C18, 130 Å, 3.5 μm, 50×4.6 mm. Conditions: 5 mM NH₄HCO₃ in water [eluent A], MeCN [eluent B] (Flow 1.0 mL/min). Gradient: 5-95% B in 1.7 min, hold at 95% for 2 min; column temp 40° C.
Method 18: Agilent 1260 (Waters Acquity PDA 210-400 nm and Waters Acquity SQ detector). Column: Waters Acquity BEH C18, 130 Å, 1.7 μm, 50×2.1 mm. Conditions: 0.1% TFA in water [eluent A], MeCN [eluent B] (Flow 0.6 mL/min). Gradient: 5-95% B in 2.2 min, hold at 95% for 1.2 min; column temp 40° C.
Method 19: Agilent 1260 (Waters Acquity PDA 210-400 nm and Waters Acquity SQ detector). Column: Waters Acquity XBridge C18, 130 Å, 3.5 μm, 50×4.6 mm. Conditions: 0.1% formic acid in water [eluent A], MeCN [eluent B] (Flow 1.0 mL/min). Gradient: 5-95% B in 2.2 min, hold at 95% for 1.2 min; column temp 40° C.

Preparative HPLC was performed using Shimadzu SIL 10AP.

### Abbreviations

| | |
|---|---|
| BINAP | 2,2′-Bis(diphenylphosphino)-1,1′-binaphthyl |
| Boc | tert-Butyloxycarbonyl |
| Cs₂CO₃ | Cesium carbonate |
| DCM | Dichloromethane |
| DIPEA | N,N-Diisopropylethylamine |
| DMF | N,N-Dimethylformamide |
| DMSO | Dimethylsulfoxide |
| DPPA | Diphenyl phosphoryl azide |
| EtOAc | Ethyl acetate |
| h | Hour (s) |
| HATU | N-[(Dimethylamino)-1H-1,2,3-triazolo- |
| | [4,5-b]pyridin-1-ylmethylene]- |
| | N-methylmethanaminium |
| | hexafluorophosphate N-oxide |
| HCl | Hydrogen chloride |
| HPLC | High Performance Liquid Chromatography |
| IrCl₂(COD) | Bis(1,5-cyclooctadiene)diiridium(I) dichloride |
| K₃PO₄ | Potassium phosphate |
| LCMS | Liquid Chromatography Mass Spectrometry |
| LDA | Lithium diisopropylethylamide |
| MeCN | Acetonitrile |
| MeOH | Methanol |
| MgSO₄ | Magnesium sulfate |
| min | Minute(s) |
| NaHCO₃ | Sodium bicarbonate |
| Na₂SO₄ | Sodium sulfate |
| NH₄HCO₃ | Ammonium bicarbonate |
| NH₄Cl | Ammonium chloride |
| NMR | Nuclear Magnetic Resonance |
| Pd₂(dba)₃ | Tris(dibenzylideneacetone)dipalladium(0) |
| Pd(dppf)Cl₂ | 1,1′-bis(diphenylphosphino)ferrocenepalladium(II) |
| | dichloride |
| PdCl₂(dppf)DCM | 1,1′-bis(diphenylphosphino)ferrocenepalladium(II) |
| | dichloride complex with DCM |
| PdCl₂(PPh₃)₂ | Bis(triphenylphosphine)palladium(II) dichloride |
| RT | Room temperature |
| s | Second (s) |
| SFC | Supercritical Fluid Chromatography |
| T3P | 1-Propylphosphonic anhydride |
| tBuBrettPhos Pd | [(2-Di-tert-butylphosphino-3,6- |
| G3 | dimethoxy-2′,4′,6′-triisopropyl-1,1′- |
| | biphenyl)-2-(2′-amino-1,1′- |
| | biphenyl)]palladium(II) methanesulfonate |
| TFA | Trifluoroacetic acid |
| THF | Tetrahydrofuran |
| X-Phos | 2-Dicyclohexylphosphino-2′,4′,6′- |
| | triisopropylbiphenyl |
| Xanthphos | 4,5-Bis(diphenylphosphino)-9,9-dimethylxanthene |

### Intermediate Synthesis

### Intermediate 1:8-Bromo-3,4-dihydro-2H-benzo[b] [1,4]oxazine-2-carbonitrile

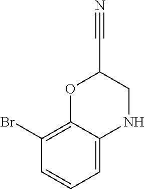 [SMILES: N#CC1CNc2cccc(Br)c2O1]

To a stirred solution of 2-amino-6-bromophenol (1.00 g, 5.32 mmol, CAS 28165-50-6) in DMSO (15 mL) was added potassium carbonate (1.47 g, 10.6 mmol). The reaction mixture was cooled to 0° C. and 2-chloroacrylonitrile (0.70 g, 7.95 mmol, CAS 920-37-6) was added dropwise. The mixture was then heated at 80° C. for 16 h. The mixture was diluted with ice cold water and extracted with ethyl acetate. The combined organic layers were dried over anhydrous Na₂SO₄, filtered and concentrated under reduced pressure. The crude product was purified by flash column chromatography (silica gel, 100-200 mesh, eluting 20-30% ethyl acetate in hexane) to provide the title compound (0.30 g). LCMS (Method 1): 2.00 min, 239.1 [M+H]⁺.

### Intermediate 2: tert-Butyl 5-(pyridin-3-yl)-3.4-dihydroquinoline-1 (2H)-carboxylate

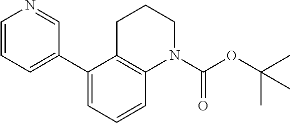 [SMILES: CC(C)(C)OC(=O)N1CCCc2c(-c3cccnc3)cccc21]

To a stirred solution of tert-butyl 5-bromo-3,4-dihydroquinoline-1 (2H)-carboxylate (0.2 g, 0.64 mmol, CAS 1706449-47-9) in THF: water (3:1, 5 mL), were added pyridin-3-ylboronic acid (79 mg, 0.64 mmol, CAS 1692-25-7), Cs₂CO₃ (0.29 g, 0.96 mmol) and the resulting reaction mass was purged under N₂ for 10 minutes. PdCl₂ (PPh₃)₂ (0.021 g, 0.035 mmol), Xanthphos (17 mg, 0.035 mmol) and purged with N₂ for 10 minutes. The mixture was heated at 90° C. for 16 h. The mixture was diluted with water, extracted with EtOAc. The combined organic layers were washed with saturated solution of NaHCO₃, brine, dried over anhydrous Na₂SO₄, filtered and concentrated under reduced pressure. The crude product was purified by flash column chromatography (silica gel, 100-200 mesh, eluting 10-25% ethyl acetate in petroleum ether) to provide the title compound (0.17 g). LCMS (Method 1): 1.73 min, 311.4 [M+H]⁺.

### Intermediate 3:5-(Pyridin-3-yl)-1,2,3,4-tetrahydroquinoline hydrochloride

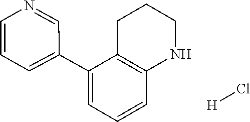 [SMILES: c1cncc(-c2cccc3c2CCCN3)c1 ; Cl]

A stirred solution of Intermediate 2 (0.17 g, 0.54 mmol) in dioxane (2 mL) was cooled to 0° C. and 4M HCl in dioxane (10 mL) was added and stirred at RT for 16 h. The mixture was concentrated under reduced pressure, triturated with n-pentane to provide the title compound (0.11 g). LCMS (Method 1): 1.06 min, 211.2 [M+H]⁺.

### Intermediate 4:2-(Quinolin-5-yl) oxazole

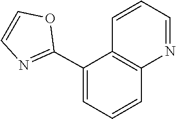 [SMILES: c1cc(-c2ncco2)c2cccnc2c1]

To a stirred solution of quinolin-5-ylboronic acid (1.00 g, 5.78 mmol, CAS 355386-94-6) in dioxane:water (5:1 ratio, 20 mL) was added 2-bromooxazole (1.28 g, 8.67 mmol, CAS 125533-82-6) and potassium carbonate (1.60 g, 11.6 mmol) and the mixture was purged with argon for 15 min. Tetrakis (triphenylphosphine) palladium (0) (0.34 g, 0.29 mmol) was added and purged with argon for 10 min. The reaction mixture was heated to 80° C. for 4 h. The reaction mixture was diluted with water and extracted with ethyl acetate. The organic layers were dried over anhydrous Na₂SO₄, filtered and concentrated under reduced pressure. The crude product was purified by flash column chromatography (silica gel, 100-200 mesh, eluting 20-40% ethyl acetate in hexane) to provide the title compound (0.8 g). LCMS (Method 1): 1.20 min, 197.6 [M+H]⁺.

### Intermediate 5:2-(1,2,3,4-Tetrahydroquinolin-5-yl) oxazole

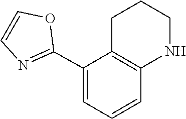 [SMILES: c1cc2c(c(-c3ncco3)c1)CCCN2]

To a stirred solution of Intermediate 4 (0.50 g, 2.55 mmol) in MeOH (10 mL) in a steel pressure vessel was added platinum (IV) oxide (29 mg, 0.13 mmol) and stirred under hydrogen pressure (50 psi) for 16 h at room temperature. The reaction mixture was passed through a celite pad, washed with MeOH and the filtrate was concentrated under reduced pressure. The crude product was purified by flash column chromatography (silica gel, 100-200 mesh silica gel, eluting 15-25% ethyl acetate in petroleum ether) to provide the title compound (0.15 g). LCMS (Method 1): 1.28 min, 201.2 [M+H]⁺.

### Intermediate 6:2-(Quinolin-5-yl) thiazole

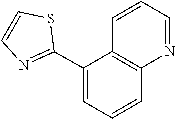 [SMILES: c1cc(-c2nccs2)c2cccnc2c1]

The title compound (0.75 g) was prepared in an analogous manner to Intermediate 4 from quinolin-5-ylboronic acid (1.00 g, 5.78 mmol, CAS 355386-94-6), 2-bromothiazole (1.42 g, 8.67 mmol, CAS 3034-53-5), potassium carbonate (1.60 g, 11.6 mmol), tetrakis(triphenylphosphine) palladium (0) (0.34 g, 0.29 mmol) in dioxane:water (4:1 ratio, 20 mL); purified by flash column chromatography (silica gel, 100-200 mesh, eluting 25-40% ethyl acetate in hexane). LCMS (Method 1): 1.26 min, 213.0 [M+H]⁺.

### Intermediate 7:2-(1.2.3,4-Tetrahydroquinolin-5-yl) thiazole

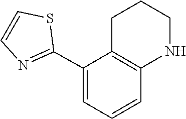 [SMILES: c1cc2c(c(-c3nccs3)c1)CCCN2]

To a stirred solution of Intermediate 6 (0.50 g, 2.35 mmol) in MeOH (10 mL) in a steel pressure vessel was added IrCl₂ (COD) (79 mg, 0.12 mmol) and stirred under hydrogen pressure (50 psi) for 48 h at room temperature. The reaction mixture was passed through a celite pad, washed with MeOH and the filtrate was concentrated under reduced pressure. The crude product was purified by flash column chromatography (silica gel, 100-200 mesh silica gel, eluting 20-35% ethyl acetate in petroleum ether) to provide the title compound (0.20 g). LCMS (Method 1): 1.40 min, 217.2 [M+H]⁺.

### Intermediate 8: (E)-N-(3-Bromophenyl)-2-methylbut-2-enamide

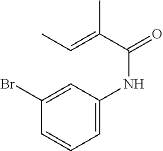

To a stirred solution of 3-bromoaniline (10 g, 58.1 mmol CAS 591-19-5) in DMF (100 mL) was added (E)-2-methylbut-2-enoic acid (5.8 g, 58.1 mmol, CAS 13201-46-2) followed by HATU (33 g, 87.2 mmol) and DIPEA (30 mL, 174 mmol), then the mixture was stirred at RT for 16 h. The reaction mixture was diluted with water and extracted with ethyl acetate. The organic layers were dried over anhydrous Na₂SO₄, filtered and concentrated under reduced pressure. The crude product was purified by flash column chromatography (silica gel, 100-200 mesh silica gel, eluting 15% ethyl acetate in petroleum ether) to provide the title compound (7.5 g). LCMS (Method 1): 1.90 min, 254.2 [M+H]⁺.

### Intermediate 9:5-Bromo-3,4-dimethyl-3,4-dihydroquinolin-2 (1H)-one

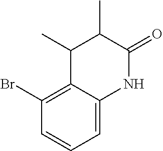

Intermediate 8 (7.5 g, 58.1 mmol) and aluminium trichloride (7.88 g, 59.0 mmol) were heated at 180° C. for 2 h. The mixture was cooled at 0° C. then quenched with 1.5 M hydrochloric acid, diluted with water and extracted with DCM. The organic layers were dried over anhydrous Na₂SO₄, filtered and concentrated under reduced pressure. The crude product was purified by preparative SFC (Chiralpak IE, 30×250 mm×5 μm, flow rate: 90 g/min, 80% CO₂ with 20% MeOH containing 0.2% diethylamine, back pressure 100 bar, Temp 35° C.) to provide the title compound (0.60 g). LCMS (Method 1): 1.85 min, 254.1 [M+H]⁺.

### Intermediate 10:5-Bromo-3,4-dimethyl-1,2,3,4-tetrahydroquinoline

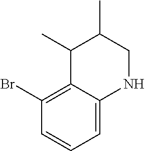

To a solution of Intermediate 9 (0.3 g, 1.2 mmol) in THF (5 mL) cooled at 0° C. was added borane in THF (1 M, 7.2 mL, 7.2 mmol) then the mixture was allowed to RT for 16 h. The mixture was quenched by addition of MeOH at 0° C. then concentrated under reduced pressure. The residue was partitioned with water and EtOAc, organic layer washed with brine, dried over Na₂SO₄, filtered and concentrated under reduced pressure to provide the title compound (0.27 g). LCMS (Method 1): 2.31 min, 240.1 [M+H]⁺.

### Intermediate 11: tert-Butyl 5-(4,4,5,5-tetramethyl-1,3,2-dioxaborolan-2-yl)-3,4-dihydroquinoline-1 (2H)-carboxylate

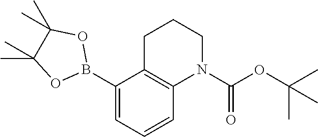 [SMILES: CC(C)(C)OC(=O)N1CCCc2c(B3OC(C)(C)C(C)(C)O3)cccc21]

To a stirred solution of tert-butyl 5-bromo-1,2,3,4-tetrahydroquinoline-1-carboxylate (2.5 g, 8.02 mmol, CAS 1706449-47-9) and bis(pinacolato)diboron (2.03 g, 8.02 mmol) in 1,4-dioxane (50 mL) was added potassium acetate (1.57 g, 16.0 mmol). The reaction mixture was degassed with argon for 10 min. Pd(dppf)Cl₂ (0.29 g, 0.40 mmol) was added and degassed with argon for 5 min. The mixture was then heated at 110° C. for 16 h. The mixture was diluted with water and extracted with ethyl acetate. The combined organic layers were dried over anhydrous Na₂SO₄, filtered and concentrated under reduced pressure. The crude product was purified by flash column chromatography (silica gel, 100-200 mesh, eluting 5-7% ethyl acetate in hexane) to provide the title compound (1.80 g). LCMS (Method 1): 2.64 min, 260.5 [M-Boc+H]⁺.

### Intermediate 12: tert-Butyl 5-(1,3,4-thiadiazol-2-yl)-3,4-dihydroquinoline-1 (2H)-carboxylate

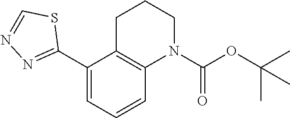 [SMILES: CC(C)(C)OC(=O)N1CCCc2c(-c3nncs3)cccc21]

To a stirred solution of Intermediate 11 (1.8 g, 5.01 mmol) 2-bromo-1,3,4-thiadiazole (0.99 g, 6.01 mmol, CAS 61929-24-6) in THF (40 ml) was added K₃PO₄ (3.32 g, 15.0 mmol) at RT. The reaction mixture was degassed with Argon for 10 min then palladium acetate (0.06 g, 0.25 mmol) and Xanthphos (0.14 g, 0.25 mmol) were added and degassed for 5 min. The mixture was stirred at 110° C. for 16 h. The mixture was cooled, diluted with water and extracted with ethyl acetate. The combined organic layers were dried over anhydrous Na₂SO₄, filtered and concentrated under reduced pressure. The crude product was purified by flash column chromatography (silica gel, 100-200 mesh, eluting 10-15% ethyl acetate in hexane) to provide the title compound (0.35 g). LCMS (Method 1): 2.33 min, 318.3 [M+H]⁺.

### Intermediate 13:2-(1,2,3,4-Tetrahydroquinolin-5-yl)-1,3,4-thiadiazole

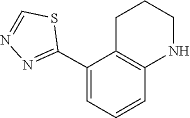 [SMILES: c1cc2c(c(-c3nncs3)c1)CCCN2]

To a stirred solution of Intermediate 12 (0.35 g, 1.10 mmol) in DCM (10 mL) at 0° C. was added 4M HCl in dioxane (5 mL). The mixture was stirred at RT for 6 h. The mixture was concentrated under reduced pressure then the residue partitioned between saturated aqueous NaHCO₃ and EtOAc. The combined organic layers were dried over anhydrous Na₂SO₄, filtered and concentrated under reduced pressure. The crude product was purified by flash column chromatography (silica gel, 100-200 mesh, eluting 30-35% ethyl acetate in hexane) to provide the title compound (0.15 g). LCMS (Method 1): 1.16 min, 218.4 [M+H]⁺.

### Intermediate 14:4-Bromo-2,2-dimethyl-2,3-dihydro-1H-inden-1-one

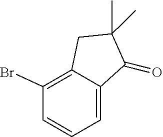

To a stirred solution of 4-bromo-1-indanone (5.0 g, 23.7 mmol, CAS 15115-60-3) in DMF (50 mL) was added sodium hydride (60% dispersion in mineral oil, 1.25 g, 52.1 mmol) and the mixture was stirred at RT for 1 h. The mixture was cooled at 0° C. then methyl iodide (10.3 mL, 166 mmol) was added and the mixture stirred at RT for 2 h. The mixture was quenched with water and extracted with EtOAc. The combined organic layers were dried over anhydrous Na₂SO₄, filtered and concentrated under reduced pressure. The crude product was purified by flash column chromatography (silica gel, 230-400 mesh, eluting 5-10% ethyl acetate in hexane) to provide the title compound (2.70 g). LCMS (Method 1): 2.14 min, 239.2 [M+H]⁺.

### Intermediate 15:5-Bromo-3,3-dimethyl-3,4-dihydroquinolin-2 (1H)-one

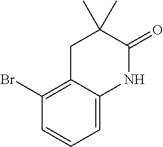

To a stirred solution of Intermediate 14 (2.7 g, 11.3 mmol) in benzene (40 mL) was added 18 M sulfuric acid (0.9 mL, 16.9 mmol) dropwise at RT. Sodium azide (3.67 g, 56.4 mmol) was added and the mixture was stirred at 70° C. for 30 min. The mixture was cooled and concentrated under reduced pressure. The residue was partitioned with water and EtOAc. The combined organic layers were washed with brine, dried over anhydrous Na₂SO₄, filtered and concentrated under reduced pressure. The crude product was purified by flash column chromatography (silica gel, 230-400 mesh, eluting 5-10% ethyl acetate in hexane) to provide the title compound (0.70 g). LCMS (Method 1): 2.21 min, 254.1 [M+H]⁺.

### Intermediate 16:5-Bromo-3,3-dimethyl-1,2,3,4-tetrahydroquinoline

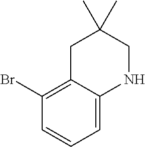

To a solution of Intermediate 15 (0.70 g, 2.75 mmol) in THF (20 mL) cooled at 0° C. was added borane dimethyl sulfide complex (2 M in THF, 6.9 mL, 13.8 mmol) then the mixture was allowed to RT for 16 h. The mixture was quenched by addition of MeOH then concentrated under reduced pressure. The residue was partitioned with water and EtOAc, organic layer washed with brine, dried over Na₂SO₄, filtered and concentrated under reduced pressure. The crude product was purified by flash column chromatography (silica gel, 230-400 mesh, eluting 5-10% ethyl acetate in hexane) to provide the title compound (0.48 g). LCMS (Method 1): 2.33 min, 240.3 [M+H]⁺.

### Intermediate 17:8-Bromo-1-methyl-1,2,3,4-tetrahydroquinoxaline

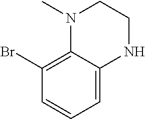

To a stirred solution of 6-bromo-N1-methylbenzene-1,2-diamine (0.5 g, 2.49 mmol, CAS 1150102-47-8) in DMA (15 mL) was added NaHCO₃ (2.09 g, 24.9 mmol) and stirred for 30 min. Then 1,2-dibromoethane (0.56 g, 2.98 mmol) was added and the mixture was heated to reflux for 16 h. The mixture was cooled and diluted with brine and EtOAc. The aqueous was extracted with ethyl acetate then the organic layer washed with brine, dried over Na₂SO₄, filtered and concentrated under reduced pressure. The crude product was purified by flash column chromatography (silica gel, 100-200 mesh, eluting 15-20% ethyl acetate in hexane) to provide the title compound (0.3 g). LCMS (Method 1): 1.21 min, 227.1 [M+H]⁺.

### Intermediate 18: tert-Butyl 8-bromo-2-cyano-2,3-dihydro-4H-benzo[b][1,4]oxazine-4-carboxylate

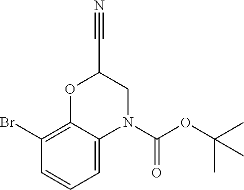 [SMILES: CC(C)(C)OC(=O)N1CC(C#N)Oc2c(Br)cccc21]

To a stirred solution of Intermediate 1 (0.89 g, 3.63 mmol) and triethylamine (0.55 g, 5.44 mmol) in THF (10 mL) was added di-tert-butyl dicarbonate (1.58 g, 7.26 mmol) and DMAP (44 mg, 0.36 mmol) and stirred at RT for 14 h. The solvent was removed under reduced pressure and the residue was partitioned with water and EtOAc. The organic layer was dried over Na₂SO₄, filtered and concentrated under reduced pressure to provide the title compound (0.69 g). LCMS (Method 1): 2.19 min, 337.1 [M−H⁻];

### Intermediate 19: tert-Butyl 8-bromo-2-cyano-2-methyl-2,3-dihydro-4H-benzo[b][1,4]oxazine-4-carboxylate

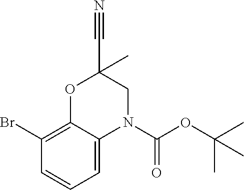 [SMILES: CC(C)(C)OC(=O)N1CC(C)(C#N)Oc2c(Br)cccc21]

To a stirred solution of Intermediate 18 (0.69 g, 2.03 mmol) in DMF (7 mL) was added sodium hydride (60% dispersion in mineral oil, 0.25 g, 6.15 mmol) and the mixture was stirred at RT for 15 min. Then methyl iodide (2.9 g, 20.3 mmol) was added and the mixture stirred at RT for 5 h. The solvent was removed under reduced pressure and the residue was partitioned with water and EtOAc. The organic layer was washed with brine, dried over Na₂SO₄, filtered and concentrated under reduced pressure to provide the title compound (0.62 g). LCMS (Method 1): 2.26 min, 253.1 [M-Boc+H]⁺.

### Intermediate 20:8-Bromo-2-methyl-3,4-dihydro-2H-benzo[b] [1,4]oxazine-2-carbonitrile hydrochloride

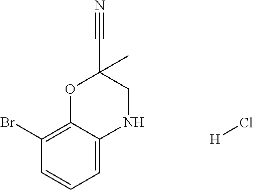

Intermediate 19 (0.35 g, 1.10 mmol) was cooled at 0° C. then 4M HCl in dioxane (6 mL) was added. The mixture was stirred at RT for 3 h. The mixture was concentrated under reduced pressure to provide the title compound (0.46 g). LCMS (Method 1): 1.84 min, 253.1 [M+H]⁺.

### Intermediate 21:3-Amino-5-bromopyridin-4-ol

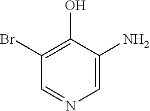

To a solution of 3-bromo-5-nitropyridin-4-ol (10.0 g, 45.6 mmol, CAS 31872-65-8) in ethanol: water (4:1, 100 mL) was added iron powder (12.7 g, 228 mmol) followed by ammonium chloride (12.2 g, 228 mmol). The mixture was stirred at 80° C. for 16 h. The mixture was concentrated under reduced pressure then the crude product was purified by flash column chromatography (neutral alumina, eluting 20-30% MeOH in DCM) to provide the title compound (8 g). LCMS (Method 1): 0.33 min, 188.9 [M+H]⁺.

### Intermediate 22:8-Bromo-2.2-dimethyl-2H-pyrido[4,3-b][1,4]oxazin-3 (4H)-one

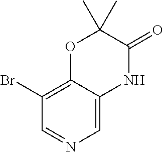

To a solution of Intermediate 21 (8.0 g, 42.3 mmol) in acetonitrile (80 mL) at cooled at 0° C. was added potassium carbonate (35.1 g, 254 mmol) and 2-bromo-2-methylpropanoyl bromide (11.7 g, 50.8 mmol) in a sealed tube. The mixture was stirred at 80° C. for 16 h. The mixture was cooled, quenched with water and extracted with ethyl acetate. The organic layer was dried over sodium sulfate, filtered and concentrated under reduced pressure. The crude product was purified by flash column chromatography (silica gel, 230-400 mesh, eluting 45-50% EtOAc in hexane) to provide the title compound (5.0 g). LCMS (Method 1): 1.44 min, 257.0 [M+H]⁺.

### Intermediate 23:8-Bromo-2,2-dimethyl-3,4-dihydro-2H-pyrido[4,3-b] [1,4]oxazine

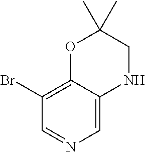

To a solution of Intermediate 22 (5.0 g, 19.5 mmol) in THF (50 mL) cooled at 0° C. was added borane in THF (1 M, 17.5 mL, 17.5 mmol) then the mixture was allowed to RT for 2 h. The mixture was quenched by addition of MeOH, which was then heated at 80° C. for 6 h then concentrated under reduced pressure. The residue was partitioned with water and EtOAc, then the organic layer was dried over Na₂SO₄, filtered and concentrated under reduced pressure. The crude product was purified by flash column chromatography (silica gel, 230-400 mesh, eluting 15-20% EtOAc in hexane) to provide the title compound (3.5 g). LCMS (Method 1): 1.10 min, 243.1 [M+H]⁺.

### Intermediate 24: 4-(tert-Butyl)₂-ethyl 8-bromo-2,3-dihydro-4H-benzo[b][1 4]oxazine-2,4-dicarboxylate

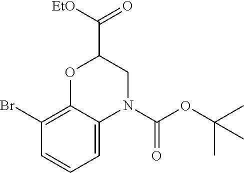 [SMILES: CCOC(=O)C1CN(C(=O)OC(C)(C)C)c2cccc(Br)c2O1]

To a solution of ethyl 8-bromo-3,4-dihydro-2H-benzo[b][1,4]oxazine-2-carboxylate (3.70 g, 12.9 mmol, CAS 1021859-84-6) and triethylamine (4.5 mL, 32.3 mmol) in THF (35 mL) was added di tert-butyl dicarbonate (8.47 g, 38.8 mmol) and DMAP (0.16 g, 1.29 mmol). The mixture was stirred at RT for 14 h. The mixture was diluted with water and extracted with ethyl acetate. The organic layer was dried over sodium sulfate, filtered and concentrated under reduced pressure. The crude product was purified by flash column chromatography (silica gel, 100-200 mesh, eluting 2-5% EtOAc in petroleum ether) to provide the title compound (4.4 g). LCMS (Method 1): 2.31 min, 286.2 [M-Boc+H]⁺.

### Intermediate 25: tert-Butyl 8-bromo-2-(hydroxymethyl)-2,3-dihydro-4H-benzo[b] [1.4]oxazine-4-carboxylate

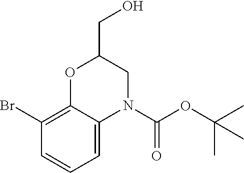 [SMILES: CC(C)(C)OC(=O)N1CC(CO)Oc2c(Br)cccc21]

To a solution of Intermediate 24 (4.35 g, 11.3 mmol) in ethanol (40 mL) at 0° C. was added sodium borohydride (1.28 g, 33.8 mmol). The mixture was stirred at RT for 14 h. The mixture was diluted with saturated aqueous ammonium chloride and extracted with ethyl acetate. The organic layer was dried over sodium sulfate, filtered and concentrated under reduced pressure. The crude product was purified by flash column chromatography (silica gel, 100-200 mesh, eluting 5-7% EtOAc in petroleum ether) to provide the title compound (3.8 g). LCMS (Method 1): 2.00 min, 244.1 [M-Boc+H]⁺.

### Intermediate 26: tert-Butyl 8-bromo-2-formyl-2,3-dihydro-4H-benzo[b] [1,4]oxazine-4-carboxylate

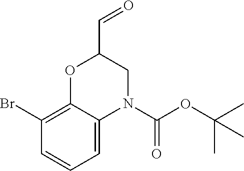 [SMILES: CC(C)(C)OC(=O)N1CC(C=O)Oc2c(Br)cccc21]

To a solution of oxalyl chloride (1.0 mL, 11.7 mmol) in DCM (10 mL) cooled at −78° C. was added DMSO (1.65 mL, 23.24 mmol) dropwise and the mixture was stirred for 1 h. Then a solution of Intermediate 25 (1.0 g, 2.91 mmol) in DCM (3 mL) was added dropwise at −78° C. and stirred for 1 h. Triethylamine (4.05 mL, 29.1 mmol) was added at −78° C. then allowed to RT for 1 h. The mixture was diluted with water and extracted with DCM. The organic layer was dried over sodium sulfate, filtered and concentrated under reduced pressure to provide the title compound (1.07 g). LCMS (Method 1): 1.81 min, 242.1 [M-Boc+H]⁺.

### Intermediate 27: tert-Butyl 8-bromo-2-ethynyl-2,3-dihydro-4H-benzo[b][1,4]oxazine-4-carboxylate

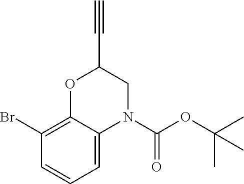 [SMILES: C#CC1CN(C(=O)OC(C)(C)C)c2cccc(Br)c2O1]

To a solution of Intermediate 26 (1.0 g, 2.92 mmol) in MeOH (10 mL) was added dimethyl(1-diazo-2-oxopropyl)phosphonate (10% in MeCN, 0.67 g, 3.50 mmol) and potassium carbonate (0.81 g, 5.84 mmol). The mixture was stirred at 60° C. for 45 min under microwave irradiation. The mixture was concentrated under reduced pressure and the residue was diluted with water and extracted with ethyl acetate. The organic layer was dried over sodium sulfate, filtered and concentrated under reduced pressure. The crude product was purified by flash column chromatography (silica gel, 100-200 mesh, eluting 2-4% EtOAc in petroleum ether) to provide the title compound (0.30 g). LCMS (Method 1): 2.25 min, 238.2 [M-Boc+H]⁺.

### Intermediate 28: 8-Bromo-2-ethynyl-3,4-dihydro-2H-benzo[b][1 4]oxazine hydrochloride

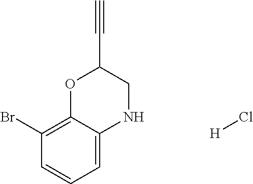

Intermediate 27 (0.29 g, 0.86 mmol) was cooled at 0° C. then 4 M HCl in dioxane (3 mL) was added. The mixture was stirred at RT for 1 h. The mixture was concentrated under reduced pressure to provide the title compound (0.22 g). LCMS (Method 1): 1.80 min, 238.2 [M+H]⁺.

### Intermediate 29:5-Cyclopropyl-1,7-naphthyridine

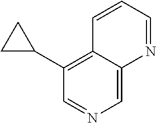 [SMILES: c1cnc2cncc(C3CC3)c2c1]

To a solution of 5-bromo-1,7-naphthyridine (0.2 g, 0.96 mmol, CAS 17965-76-3) and cyclopropylboronic acid (82 mg, 0.95 mmol) in 1,4-dioxane:water (8:1, 4.5 mL) was added potassium carbonate (0.4 g, 2.87 mmol) and the mixture was degassed with nitrogen for 10 min. PdCl₂ (dppf) DCM (78 mg, 0.095 mmol) was added and the mixture degassed with nitrogen for 15 min then stirred at 90° C. for 16 h. The mixture was diluted with ethyl acetate, filtered through Celite. The filtrate was washed with water, brine, dried over sodium sulfate, filtered and concentrated under reduced pressure. The crude product was purified by flash column chromatography (silica gel, 100-200 mesh, eluting 50% EtOAc in petroleum ether) to provide the title compound (0.15 g). LCMS (Method 1): 1.04 min, 171.2 [M+H]⁺.

### Intermediate 30:5-Cyclopropyl-1,2,3,4-tetrahydro-1,7-naphthyridine

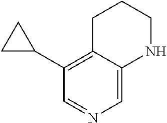 [SMILES: c1ncc(C2CC2)c2c1NCCC2]

To a stirred solution of Intermediate 29 (0.15 g, 0.88 mmol) in ethanol (20 mL) was added platinum (IV) oxide (20 mg, 0.09 mmol) and stirred under an atmosphere of hydrogen for 16 h at RT. The reaction mixture was concentrated under reduced pressure, diluted with EtOAc and filtered through Celite. The filtrate was dried over sodium sulfate, filtered and concentrated under reduced pressure to provide the title compound (0.14 g). LCMS (Method 1): 1.09 min, 175.3 [M+H]⁺.

### Intermediate 31:8-Cyclopropyl-2,2-dimethyl-2H-pyrido[4,3-b][1,4]oxazin-3 (4H)-one

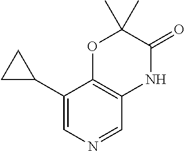 [SMILES: CC1(C)Oc2c(cncc2C2CC2)NC1=O]

To a solution of Intermediate 22 (1.5 g, 5.84 mmol) and 2-cyclopropyl-4,4,5,5-tetramethyl-1,3,2-dioxaborolane (1.47 g, 8.75 mmol) in 1,4-dioxane:water (4:1, 15 mL) was added potassium carbonate (2.01 g, 14.6 mmol) and the mixture was degassed with nitrogen for 5 min. PdCl₂ (dppf) DCM (0.71 g, 0.88 mmol) was added and the mixture stirred at 80° C. for 16 h. The mixture was filtered through Celite and the filtrate was concentrated under reduced pressure. The residue was diluted with EtOAc and washed with water, brine, dried over sodium sulfate, filtered and concentrated under reduced pressure. The crude product was purified by flash column chromatography (silica gel, 230-400 mesh, eluting 30-40% EtOAc in petroleum ether) to provide the title compound (0.65 g). LCMS (Method 2): 0.96 min, 219.2 [M+H]⁺.

### Intermediate 32:8-Cyclopropyl-2.2-dimethyl-3,4-dihydro-2H-pyrido[4,3-b][1,4]oxazine

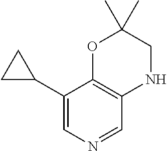

To a solution of Intermediate 31 (0.6 g, 2.75 mmol) in THF (10 mL) at 0° C. was added lithium aluminium hydride (2 M in THF, 4.12 mL, 8.25 mmol). The mixture was stirred at RT for 3 h. The mixture was concentrated under reduced pressure and the residue was diluted with water and extracted with ethyl acetate. The organic layer was dried over sodium sulfate, filtered and concentrated under reduced pressure. The crude product was purified by preparative-HPLC (X SELECT C18, 25×150 mm×10 μm, flow rate: 18 mL/min, 10 mM NH₄HCO₃ in water with MeCN 35% to 65% over 8 min, held at 65% for 1.7 min,) to provide the title compound (0.10 g). LCMS (Method 1): 1.36 min, 205.1 [M+H]⁺.

### Intermediate 33: (3-Bromo-5-nitropyridin-4-yl)-L-proline

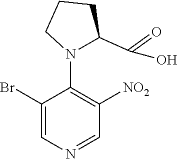 [SMILES: O=C(O)[C@@H]1CCCN1c1c(Br)cncc1[N+](=O)[O-]]

To a solution of 3-bromo-4-chloro-5-nitropyridine (2.0 g, 8.43 mmol, CAS 31872-63-6) and L-proline (1.26 g, 10.9 mmol) in acetonitrile (20 mL) at 0° C. was added triethylamine (3.65 mL, 25.3 mmol). The mixture was stirred at RT for 2 h. The mixture was filtered through Celite and filtrate concentrated under reduced pressure. The crude product was purified by flash column chromatography (silica gel, 100-200 mesh, eluting 5-10% MeOH in DCM) to provide the title compound (2.1 g). LCMS (Method 2): 1.43 min, 316.2 [M+H]⁺.

### Intermediate 34: (S)-1-Bromo-6a,7,8,9-tetrahydropyrido[3,4-e] pyrrolo[1,2-a]pyrazin-6 (5H)-one

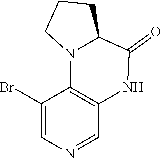 [SMILES: O=C1Nc2cncc(Br)c2N2CCC[C@@H]12]

To a solution of Intermediate 33 (2.0 g, 6.34 mmol) in acetic acid (8 mL) was added iron powder (1.06 g, 19.0 mmol). The mixture was stirred at 80° C. for 4 h. The mixture was concentrated under reduced pressure then the residue diluted with water, basified with sodium bicarbonate then extracted with 10% MeOH in DCM. The combined organic layer was dried over sodium sulfate, filtered and concentrated under reduced pressure to provide the title compound (1.1 g). LCMS (Method 1): 0.99 min, 268.2 [M+H]⁺.

### Intermediate 35: (S)-1-bromo-5,6,6a,7,8,9-hexahydropyrido[3,4-e]pyrrolo[1,2-a]pyrazine

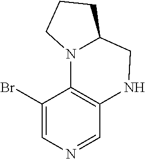 [SMILES: Brc1cncc2c1N1CCC[C@H]1CN2]

To a solution of Intermediate 34 (1.1 g, 4.10 mmol) in THF (10 mL) cooled at 0° C. was added borane in THF (1 M, 12.3 mL, 12.3 mmol) then the mixture was stirred at 80° C. for 3 h. The mixture was quenched by addition of MeOH, which was then heated at 80° C. for 3 h. The mixture was allowed to cool then 1 M aqueous HCl (5 mL) was added and heated at 80° C. for 3 h. The mixture was concentrated under reduced pressure. The residue was basified with aqueous sodium bicarbonate and extracted with 10% MeOH in DCM. The organic layer was dried over Na₂SO₄, filtered and concentrated under reduced pressure. The crude product was purified by flash column chromatography (silica gel, 230-400 mesh, eluting 50-100% EtOAc in hexane) to provide the title compound (0.5 g). LCMS (Method 1): 1.16 min, 254.1 [M+H]⁺.

### Intermediate 36: N-(3-Bromo-5-nitropyridin-4-yl)-N-methyl-D-alanine

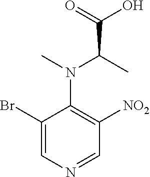 [SMILES: C[C@H](C(=O)O)N(C)c1c(Br)cncc1[N+](=O)[O-]]

To a solution of 3-bromo-4-chloro-5-nitropyridine (2.0 g, 8.42 mmol, CAS 31872-63-6) and N-methyl-D-alanine (1.30 g, 12.6 mmol) in 1,4-dioxane:water (2:1, 20 mL) at 0° C. was added sodium carbonate (1.34 g, 12.6 mmol). The mixture was stirred at RT for 16 h. The mixture was quenched with water and washed with EtOAc. The aqueous was acidified with aqueous sodium bisulfate and extracted with EtOAc. The organic layer was washed with brine, dried over sodium sulfate, filtered and concentrated under reduced pressure. The crude product was purified by flash column chromatography (silica gel, 100-200 mesh, eluting 50% EtOAc in hexane) to provide the title compound (1.25 g). LCMS (Method 1): 1.54 min, 304.3 [M+H]⁺.

### Intermediate 37: (R)-8-Bromo-1.2-dimethyl-1,4-dihydropyrido[3.4-b]pyrazin-3 (2H)-one

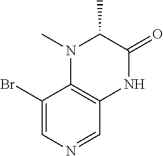

To a solution of Intermediate 36 (1.25 g, 4.11 mmol) in acetic acid (25 mL) was added iron powder (0.69 g, 12.3 mmol). The mixture was stirred at 80° C. for 4 h. The mixture was concentrated under reduced pressure then the residue diluted with water, basified with sodium bibarbonate then extracted with 10% MeOH in DCM. The combined organic layer was dried over sodium sulfate, filtered and concentrated under reduced pressure to provide the title compound (0.6 g). LCMS (Method 2): 0.80 min, 256.1 [M+H]⁺.

### Intermediate 38: (R)-8-bromo-1.2-dimethyl-1,2,3,4-tetrahydropyrido[3,4-b]pyrazine

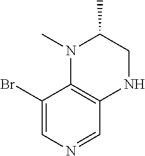

To a solution of Intermediate 37 (0.6 g, 2.34 mmol) in THF (12 mL) cooled at 0° C. was added borane in THF (1 M, 7.02 mL, 7.02 mmol) then the mixture was stirred at 80° C. for 3 h. The mixture was quenched by addition of MeOH, which was then heated at 60° C. for 16 h. The mixture was concentrated under reduced pressure. The residue was basified with aqueous sodium bicarbonate and extracted with 10% MeOH in DCM. The organic layer was dried over Na₂SO₄, filtered and concentrated under reduced pressure to provide the title compound (0.5 g). LCMS (Method 1): 1.10 min, 242.1 [M+H]⁺.

### Intermediate 39: 8′—Bromo-4,5-dihydro-2H-spiro [furan-3,2′-pyrido[4,3-b][1,4]oxazin]-3′ (4′H)-one

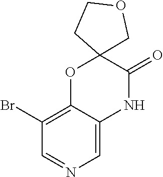 [SMILES: O=C1Nc2cncc(Br)c2OC12CCOC2]

A solution of 3-hydroxytetrahydrofuran-3-carboxylic acid (0.6 g, 4.55 mmol, CAS 183162-36-9) in thionyl chloride (6 mL) was stirred at 80° C. for 12 h. The mixture was concentrated under reduced pressure then flushed with nitrogen to give the acid chloride A. Separately, a mixture of 3-amino-5-bromopyridin-4-ol (0.6 g, 3.17 mmol) and potassium carbonate (2.63 g, 19.0 mmol) in DMF: THF (1:1, 6 mL) was stirred at 0° C. for 30 min. The acid chloride A was added and the mixture was stirred at RT for 1 h, then at 100° C. for 16 h. The mixture was cooled and concentrated under reduced pressure. This was diluted with water and extracted with ethyl acetate. The organic layer was dried over sodium sulfate, filtered and concentrated under reduced pressure. The crude product was purified by flash column chromatography (silica gel, 100-200 mesh, eluting 20-30% EtOAc in hexane) to provide the title compound (0.24 g). LCMS (Method 1): 1.35 min, 285.3 [M+H]⁺.

### Intermediate 40: 8′—Bromo-3′,4,4′,5-tetrahydro-2H-spiro [furan-3,2′-pyrido[4,3-b][1,4]oxazine]

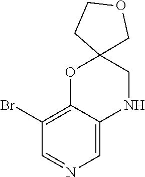 [SMILES: Brc1cncc2c1OC1(CCOC1)CN2]

To a solution of Intermediate 39 (0.24 g, 0.84 mmol) in THF (10 mL) cooled at 0° C. was added borane in THF (1 M, 2.53 mL, 2.53 mmol) then the mixture was stirred at 70° C. for 3 h. The mixture was quenched by addition of MeOH, which was then heated at 70° C. for 3 h. The mixture was concentrated under reduced pressure. The residue was diluted with aqueous sodium bicarbonate and extracted with 10% MeOH in DCM. The organic layer was dried over Na₂SO₄, filtered and concentrated under reduced pressure to provide the title compound (0.2 g). LCMS (Method 1): 0.98 min, 271.2 [M+H]⁺.

### Intermediate 41:1-(3-Bromo-5-nitropyridin-4-yl) piperidine-2-carboxylic acid

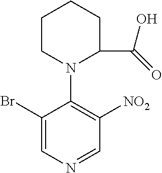 [SMILES: O=C(O)C1CCCCN1c1c(Br)cncc1[N+](=O)[O-]]

To a solution of pipecolinic acid (1.63 g, 12.6 mmol, CAS 535-75-1) in 1,4-dioxane:water (2:1, 40 mL) at 0° C. was added sodium carbonate (1.34 g, 12.6 mmol) and this was stirred for 5 min, then 3-bromo-4-chloro-5-nitropyridine (2.0 g, 8.42 mmol, CAS 31872-63-6) was added. The mixture was stirred at RT for 16 h. The mixture was acidified with aqueous sodium bisulfate and extracted with EtOAc. The organic layer was washed with brine, dried over sodium sulfate, filtered and concentrated under reduced pressure. The crude product was purified by flash column chromatography (silica gel, 100-200 mesh, eluting 30-35% EtOAc in hexane) to provide the title compound (1.5 g). LCMS (Method 1): 1.77 min, 330.2 [M+H]⁺.

### Intermediate 42:1-Bromo-7,8,9, 10-tetrahydro-5H-dipyrido[1,2-a: 3′,4′-e] pyrazin-6 (6aH)-one

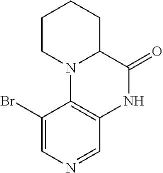 [SMILES: O=C1Nc2cncc(Br)c2N2CCCCC12]

To a solution of Intermediate 41 (1.25 g, 4.11 mmol) in acetic acid (14.5 mL) was added iron powder (0.74 g, 13.2 mmol). The mixture was stirred at 85° C. for 5 h. The mixture was basified carefully with aqueous sodium bicarbonate then extracted with EtOAc. The combined organic layer was dried over sodium sulfate, filtered and concentrated under reduced pressure to provide the title compound (0.50 g). LCMS (Method 2): 1.17 min, 282.1 [M+H]⁺.

### Intermediate 43:1-Bromo-6,6a,7,8,9,10-hexahydro-5H-dipyrido[1,2-a: 3′,4′-e] pyrazine

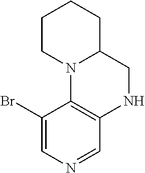 [SMILES: Brc1cncc2c1N1CCCCC1CN2]

To a solution of Intermediate 42 (0.45 g, 1.60 mmol) in THF (5 mL) cooled at 0° C. was added borane in THF (1 M, 4.79 mL, 4.79 mmol) then the mixture was stirred at 80° C. for 3 h. The mixture was quenched by addition of MeOH, which was then heated at 80° C. for 6 h. The mixture was concentrated under reduced pressure. The crude product was purified by flash column chromatography (silica gel, 100-200 mesh, eluting 30-35% EtOAc in hexane) to provide the title compound (0.16 g). LCMS (Method 1): 1.31 min, 268.3 [M+H]⁺.

### Intermediate 44: N-(3-Bromo-5-nitropyridin-4-yl)-N-methylglycine

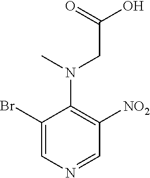 [SMILES: CN(CC(=O)O)c1c(Br)cncc1[N+](=O)[O-]]

To a solution of methyl N-methylglycinate hydrochloride (2.20 g, 15.8 mmol, CAS 13515-93-0) in acetonitrile (25 mL) at 0° C. was added triethylamine (4.43 mL, 31.6 mmol) and this was stirred for 5 min, then 3-bromo-4-chloro-5-nitropyridine (2.5 g, 10.5 mmol, CAS 31872-63-6) was added. The mixture was stirred at RT for 16 h. The mixture was diluted with water and extracted with EtOAc. The organic layer was washed with brine, dried over sodium sulfate, filtered and concentrated under reduced pressure. The crude product was purified by flash column chromatography (silica gel, 100-200 mesh, eluting 35-40% EtOAc in petroleum ether) to provide the title compound (2.0 g). LCMS (Method 1): 1.76 min, 290.9 [M+H]⁺.

### Intermediate 45:8-Bromo-1-methyl-1,4-dihydropyrido[3.4-b]pyrazin-3 (2H)-one

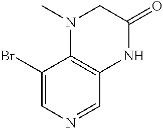

To a solution of Intermediate 44 (2.0 g, 6.89 mmol) in acetic acid (20 mL) was added iron powder (1.15 g, 20.7 mmol). The mixture was stirred at 80° C. for 16 h. The mixture was basified carefully with aqueous sodium bicarbonate then filtered through Celite. The filtrate was extracted with EtOAc. The combined organic layer was dried over sodium sulfate, filtered and concentrated under reduced pressure to provide the title compound (1.50 g). LCMS (Method 3): 0.73 min, 242.2 [M+H]⁺.

### Intermediate 46:8-Bromo-1-methyl-1,2,3,4-tetrahydropyrido[3,4-b]pyrazine

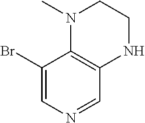

To a solution of Intermediate 45 (1.50 g, 6.19 mmol) in THF (15 mL) cooled at 0° C. was added borane in THF (1 M, 18.5 mL, 18.5 mmol) then the mixture was stirred at 80° C. for 2 h. The mixture was cooled to 0° C. then quenched by addition of MeOH, which was then heated at 80° C. for 16 h. The mixture was concentrated under reduced pressure. The crude product was purified by flash column chromatography (silica gel, 100-200 mesh, eluting 5-10% MeOH in DCM) to provide the title compound (0.35 g). LCMS (Method 4): 0.93 min, 228.2 [M+H]⁺.

### Intermediate 47: Methyl 2-((3-bromo-5-nitropyridin-4-yl)oxy) propanoate

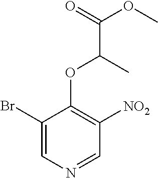 [SMILES: COC(=O)C(C)Oc1c(Br)cncc1[N+](=O)[O-]]

To a stirred suspension of sodium hydride (60% dispersion in mineral oil, 1.01 g, 25.3 mmol) in THF (25 mL) at 0° C. was added methyl 2-hydroxypropanoate (1.31 g, 12.6 mmol, CAS 547-64-8) then the mixture was stirred at RT for 30 min. Then a solution of 3-bromo-4-chloro-5-nitropyridine (2.0 g, 8.42 mmol, CAS 31872-63-6) in THF (25 mL) was added and the mixture was stirred at 80° C. for 2 h. The mixture was cooled and diluted with water and extracted with EtOAc. The organic layer was washed with brine, dried over Na₂SO₄, filtered and concentrated under reduced pressure. The crude product was purified by flash column chromatography (silica gel, 230-400 mesh, eluting 30-35% EtOAc in petroleum ether) to provide the title compound (1.0 g). LCMS (Method 4): 1.73 min, 305.2 [M+H]⁺.

### Intermediate 48:8-Bromo-2-methyl-2H-pyrido[4,3-b][1,4]oxazin-3 (4H)-one

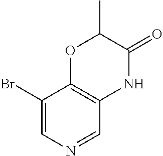

To a solution of Intermediate 47 (1.0 g, 3.28 mmol) in acetic acid (10 mL) was added iron powder (0.55 g, 9.84 mmol). The mixture was stirred at 80° C. for 2 h. The mixture was concentrated under reduced pressure then diluted with water and extracted with EtOAc. The combined organic layer was washed with brine, dried over sodium sulfate, filtered and concentrated under reduced pressure. The crude product was triturated with diethyl ether, filtered and dried in vacuum to provide the title compound (0.60 g). LCMS (Method 4): 1.30 min, 243.1 [M+H]⁺.

### Intermediate 49:8-Bromo-2-methyl-3,4-dihydro-2H-pyrido[4,3-b][1 4]oxazine

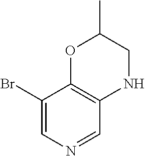

To a solution of Intermediate 48 (0.60 g, 2.47 mmol) in THF (6 mL) cooled at 0° C. was added borane in THF (1 M, 7.41 mL, 7.41 mmol) then the mixture was stirred at 80° C. for 2 h. The mixture was cooled to 0° C. then quenched by addition of MeOH, which was then heated at 80° C. for 2 h. The mixture was concentrated under reduced pressure then diluted with water and extracted with EtOAc. The organic layer was washed with brine, dried over sodium sulfate, filtered and concentrated under reduced pressure. The crude product was purified by flash column chromatography (silica gel, 230-400 mesh, eluting 40-45% EtOAc in petroleum ether) to provide the title compound (0.50 g). 1H NMR (400 MHz; CDCl₃) δ: 8.01 (s, 1H), 7.82 (s, 1H), 4.39-4.43 (m, 1H), 3.88 (br s, 1H), 3.41-3.46 (m, 1H), 3.11-3.16 (m, 1H), 1.45-1.47 (m, 3H).

### Intermediate 50: (R)-8-Bromo-1,2-dimethyl-4-((2-(trimethylsilyl) ethoxy)methyl)-1,4-dihydropyrido[3,4-b]pyrazin-3 (2H)-one

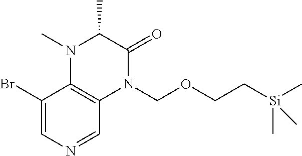 [SMILES: C[C@@H]1C(=O)N(COCC[Si](C)(C)C)c2cncc(Br)c2N1C]

To a stirred solution of Intermediate 37 (1.0 g, 3.90 mmol) in THF (20 mL) at 0° C. was added sodium hydride (60% dispersion in mineral oil, 0.46 g, 11.7 mmol) and the mixture was stirred for 15 min. To this was added (trimethylsilyl) ethoxylmethyl chloride (1.3 g, 7.81 mmol) then the mixture was stirred at RT for 2 h. The mixture was quenched with ice cold water and extracted with EtOAc. The organic layer was washed with water, brine, dried over Na₂SO₄, filtered and concentrated under reduced pressure. The crude product was purified by flash column chromatography (silica gel, 230-400 mesh, eluting 10% EtOAc in petroleum ether) to provide the title compound (0.75 g). LCMS (Method 1): 2.18 min, 386.2 [M+H]⁺.

### Intermediate 51:8-Bromo-1,2,2-trimethyl-4-((2-(trimethylsilyl) ethoxy)methyl)-1,4-dihydropyrido[3,4-b]pyrazin-3 (2H)-one

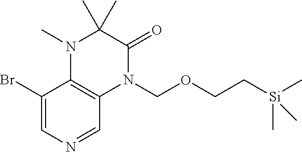 [SMILES: CN1c2c(Br)cncc2N(COCC[Si](C)(C)C)C(=O)C1(C)C]

To a stirred solution of Intermediate 50 (0.75 g, 1.94 mmol) in THF (15 mL) cooled at −40° C. was added LDA (1 M in THF, 5.82 mL, 5.82 mmol) and the mixture was stirred for 15 min. To this was added iodomethane (0.83 g, 5.82 mmol) then the mixture was stirred at −40° C. for 2 h. The mixture was quenched with ice cold water and extracted with EtOAc. The organic layer was washed with water, brine, dried over Na₂SO₄, filtered and concentrated under reduced pressure. The crude product was purified by flash column chromatography (silica gel, 230-400 mesh, eluting 8% EtOAc in petroleum ether) to provide the title compound (0.41 g). LCMS (Method 1): 2.51 min, 400.2 [M+H]⁺.

### Intermediate 52:8-Bromo-1,2,2-trimethyl-1,4-dihydropyrido[3,4-b]pyrazin-3 (2H)-one

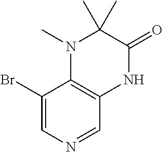

Intermediate 51 (0.40 g, 0.99 mmol) was dissolved in concentrated HCl (37% aqueous solution, 8 mL) and stirred at 60° C. for 3 h. The mixture was basified carefully with saturated aqueous sodium bicarbonate and extracted with EtOAc. The organic layer was washed with brine, dried over Na₂SO₄, filtered and concentrated under reduced pressure to provide the title compound (0.23 g). LCMS (Method 1): 1.29 min, 270.0 [M+H]⁺.

### Intermediate 53:8-Bromo-1,2.2-trimethyl-1,2,3,4-tetrahydropyrido[3,4-b]pyrazine

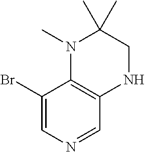

To a solution of Intermediate 52 (0.23 g, 0.85 mmol) in THF (2.3 mL) cooled at 0° C. was added borane in THF (1 M, 4.25 mL, 4.25 mmol) then the mixture was stirred at 80° C. for 3 h. The mixture was quenched by addition of MeOH, which was then heated at 60° C. for 16 h. The mixture was concentrated under reduced pressure then diluted with water and extracted with EtOAc. The organic layer was washed with brine, dried over sodium sulfate, filtered and concentrated under reduced pressure to provide the title compound (0.23 g). LCMS (Method 1): 1.13 min, 256.1 [M+H]⁺.

### Intermediate 54:3-Amino-5-chloropyridin-4-ol

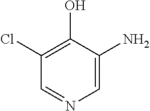

To a solution of 3-chloro-5-nitropyridin-4-ol (2.0 g, 11.5 mmol) in ethanol: water (2:1, 20 mL) was added iron powder (3.2 g, 57.3 mmol) followed by ammonium chloride (3.07 g, 57.3 mmol). The mixture was stirred at 80° C. for 16 h. The mixture was concentrated under reduced pressure. The crude product was purified by flash column chromatography (neutral alumina, eluting 20-30% MeOH in DCM) to provide the title compound (1.0 g). LCMS (Method 1): 0.28 min, 145.2 [M+H]⁺.

### Intermediate 55:8-Chloro-2,2-dimethyl-2H-pyrido[4,3-b] [1,4]oxazin-3 (4H)-one

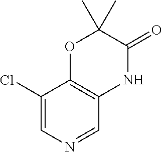

To a solution of Intermediate 54 (1.0 g, 6.92 mmol) in acetonitrile (10 mL) at 0° C. was added potassium carbonate (5.74 g, 41.5 mmol) and this was stirred for 30 min at RT. The mixture was cooled at 0° C. then 2-bromo-2-methylpropanoyl bromide (1.91 g, 8.30 mmol) was added and stirred at RT for 1 h. The mixture was concentrated at reduced pressure then diluted with water and extracted with EtOAc. The organic layer was dried over sodium sulfate, filtered and concentrated under reduced pressure to provide the title compound (0.6 g). LCMS (Method 1): 1.51 min, 212.9 [M+H]⁺.

### Intermediate 56:8-Chloro-2.2-dimethyl-3,4-dihydro-2H-pyrido[4,3-b] [1,4]oxazine

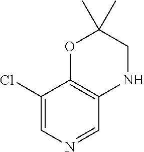

To a solution of Intermediate 55 (0.60 g, 2.82 mmol) in THF (10 mL) cooled at 0° C. was added borane in THF (1 M, 14.1 mL, 14.1 mmol) then the mixture was stirred at RT for 2 h. The mixture was quenched by addition of MeOH, which was then heated at 80° C. for 8 h. The mixture was concentrated under reduced pressure then diluted with water and extracted with EtOAc. The organic layer was dried over sodium sulfate, filtered and concentrated under reduced pressure to provide the title compound (0.45 g). LCMS (Method 1): 1.17 min, 199.0 [M+H]⁺.

### Intermediate 57:3-Bromo-5-nitro-N-(tetrahydro-2H-pyran-4-yl)pyridin-4-amine

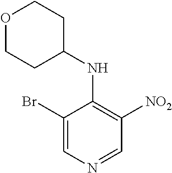 [SMILES: O=[N+]([O-])c1cncc(Br)c1NC1CCOCC1]

To a solution of 4-aminotetrahydropyran (2.55 g, 25.3 mmol, CAS 38041-19-9) in 1,4-dioxane:water (2:1, 40 mL) at 0° C. was added sodium carbonate (3.57 g, 33.7 mmol) followed by 3-bromo-4-chloro-5-nitropyridine (4.0 g, 16.8 mmol, CAS 31872-63-6). The mixture was stirred at RT for 16 h. The mixture was quenched with water and extracted with EtOAc. The organic layer was washed with brine, dried over sodium sulfate, filtered and concentrated under reduced pressure to provide the title compound (4.3 g). LCMS (Method 1): 1.66 min, 302.0 [M+H]⁺.

### Intermediate 58:5-Bromo-N4-(tetrahydro-2H-pyran-4-yl)pyridine-3,4-diamine

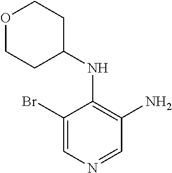 [SMILES: Nc1cncc(Br)c1NC1CCOCC1]

To a solution of Intermediate 57 (4.3 g, 14.2 mmol) in ethanol: water (4:1, 43 mL) was added iron powder (3.97 g, 71.2 mmol) followed by ammonium chloride (3.80 g, 71.2 mmol). The mixture was stirred at 80° C. for 16 h. The mixture was concentrated under reduced pressure, filtered through celite and washed 10% MeOH in DCM. The filtrate was concentrated and the crude product was purified by flash column chromatography (silica gel, 230-400 mesh eluting 5% MeOH in DCM) to provide the title compound (3.4 g). LCMS (Method 1): 0.85 min, 271.9 [M+H]⁺.

### Intermediate 59:8-Bromo-1-(tetrahydro-2H-pyran-4-yl)-1,2,3,4-tetrahydropyrido[3,4-b]pyrazine

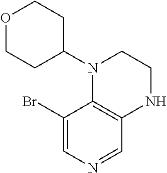 [SMILES: Brc1cncc2c1N(C1CCOCC1)CCN2]

To a solution of Intermediate 58 (3.4 g, 12.5 mmol) in ethanol (68 mL) was added oxalaldehyde (0.87 g, 15.0 mmol) then the mixture was stirred at 100° C. for 16 h. The mixture was concentrated under reduced pressure and extracted with ethyl acetate, dried over sodium sulfate, filtered and concentrated under reduced pressure. The crude material was suspended in THF (68 mL) at 0° C. then TFA (34 mL) and sodium borohydride (1.65 g, 43.7 mmol) were added. The mixture was stirred at RT for 2 h. The mixture was concentrated under reduced pressure and the crude product was purified by preparative HPLC (X-Bridge C18, 25×150 mm×10 μm, flow rate: 19 mL/min, 10 mM NH₄HCO₃ in water with MeCN 20% to 50% over 7 min, held at 50% for 2.2 min then to 98% over 3.8 min) to provide the title compound (2.0 g). LCMS (Method 1): 1.09 min, 298.1 [M+H]⁺.

### Intermediate 60: Methyl (R)-2-((3-bromo-5-nitropyridin-4-yl)(methyl)amino) butanoate

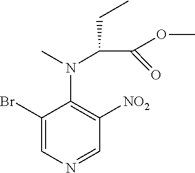 [SMILES: CC[C@H](C(=O)OC)N(C)c1c(Br)cncc1[N+](=O)[O-]]

To a solution of 3-bromo-4-chloro-5-nitropyridine (2.0 g, 8.42 mmol, CAS 31872-63-6) in 1,4-dioxane:water (2:1, 430 mL) was added sodium carbonate (3.57 g, 25.3 mmol) and stirred for 10 min at RT. Then 4 methyl (R)-2-(methylamino) butanoate (2.55 g, 25.3 mmol, CAS 745759-46-0) was added and the mixture was stirred at RT for 16 h. The mixture was quenched with water and extracted with EtOAc. The organic layer was concentrated under reduced pressure. The crude product was purified by flash column chromatography (silica gel, 60-120 mesh eluting 20% EtOAc in petroleum ether) to provide the title compound (0.35 g). LCMS (Method 1): 1.97 min, 332.5 [M+H]⁺.

### Intermediate 61: (R)-8-bromo-2-ethyl-1-methyl-1,4-dihydropyrido[3,4-b]pyrazin-3 (2H)-one

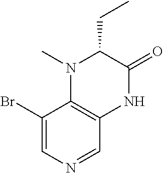

To a solution of Intermediate 60 (0.35 g, 1.06 mmol) in acetic acid (3.5 mL) was added iron powder (0.34 g, 6.04 mmol). The mixture was stirred at 80° C. for 2 h. The mixture was concentrated under reduced pressure, dissolved in 10% MeOH in DCM then filtered through Celite. The filtrate was washed with aqueous sodium bicarbonate. The combined organic layer was concentrated under reduced pressure to provide the title compound (0.32 g). LCMS (Method 4): 1.06 min, 270.2 [M+H]⁺.

### Intermediate 62: (R)-8-Bromo-2-ethyl-1-methyl-1,2,3,4-tetrahydropyrido[3,4-b]pyrazine

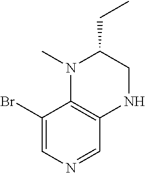

To a solution of Intermediate 61 (0.32 g, 1.19 mmol) in THF (3.2 mL) cooled at 0° C. was added borane in THF (1 M, 5.9 mL, 5.9 mmol) then the mixture was stirred at RT for 2 h. The mixture was quenched by addition of MeOH, which was then heated at 80° C. for 16 h. The mixture was concentrated under reduced pressure. The crude product was purified by flash column chromatography (neutral alumina, eluting 30-40% EtOAc in hexane) to provide the title compound (0.1 g). LCMS (Method 1): 1.23 min, 256.1 [M+H]⁺.

### Intermediate 63: Methyl N-(3-bromo-5-nitropyridin-4-yl)-N-methyl-D-valinate

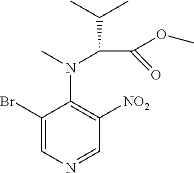 [SMILES: COC(=O)[C@@H](C(C)C)N(C)c1c(Br)cncc1[N+](=O)[O-]]

To a solution of methyl N-methyl-D-valinate (2.50 g, 17.2 mmol, CAS 769890-37-1) in 1,4-dioxane:water (4:1, 25 mL) at 0° C. was added sodium carbonate (3.57 g, 33.7 mmol). This was stirred for 10 min followed by addition of 3-bromo-4-chloro-5-nitropyridine (2.86 g, 12.1 mmol, CAS 31872-63-6). The mixture was stirred at 80° C. for 16 h. The mixture was quenched with water and extracted with EtOAc. The organic layer was washed with brine, dried over sodium sulfate, filtered and concentrated under reduced pressure. The crude product was purified by flash column chromatography (silica gel, 100-200 mesh, eluting 5-10% EtOAc in hexane) to provide the title compound (0.16 g). LCMS (Method 1): 2.13 min, 346.2 [M+H]⁺.

### Intermediate 64: (R)-8-Bromo-2-isopropyl-1-methyl-1,4-dihydropyrido[3,4-b]pyrazin-3 (2H)-one

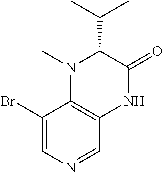 [SMILES: CC(C)[C@@H]1C(=O)Nc2cncc(Br)c2N1C]

To a solution of Intermediate 63 (0.16 g, 0.46 mmol) in acetic acid (2.0 mL) was added iron powder (0.08 g, 1.39 mmol). The mixture was stirred at 80° C. for 4 h. The mixture was filtered through Celite washing with EtOAc and the filtrate concentrated under reduced pressure to provide the title compound (0.16 g). LCMS (Method 1): 1.15 min, 284.5 [M+H]⁺.

### Intermediate 65: (R)-8-Bromo-2-isopropyl-1-methyl-1,2,3,4-tetrahydropyrido[3,4-b]pyrazine

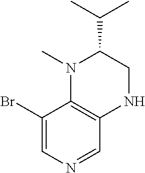

To a solution of Intermediate 64 (0.16 g, 0.56 mmol) in THF (3 mL) cooled at 0° C. was added borane in THF (1 M, 2.8 mL, 2.8 mmol) then the mixture was stirred at RT for 16 h. The mixture was quenched by addition of MeOH, which was then heated at 60° C. for 5 h. The mixture was concentrated under reduced pressure. The crude product was purified by flash column chromatography (silica gel, 100-200 mesh, eluting 50-100% EtOAc in hexane) to provide the title compound (55 mg). LCMS (Method 1): 1.32 min, 270.1 [M+H]⁺.

### Intermediate 66:3-Bromo-4-chloro-5-nitropyridin-2-amine

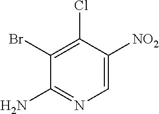 [SMILES: Nc1ncc([N+](=O)[O-])c(Cl)c1Br]

3-Bromo-4-chloropyridin-2-amine (4 g, 19.28 mmol, CAS 221297-82-1) was added in portions to concentrated sulfuric acid (98%, 40 mL) cooled at 0° C. followed by addition of nitric acid (48%, 2 mL) then the mixture was stirred at RT for 1 h. The mixture was poured into ice water and the resulting solid was filtered, washing with water and the solid dried under reduced pressure to provide the title compound (2.6 g). LCMS (Method 1): 1.59 min, 252.1 [M+H]⁺.

### Intermediate 67: Methyl 2-((2-amino-3-bromo-5-nitropyridin-4-yl)oxy)acetate

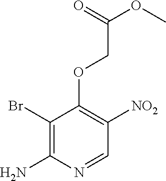 [SMILES: COC(=O)COc1c([N+](=O)[O-])cnc(N)c1Br]

To a stirred solution of methyl 2-hydroxyacetate (1.85 g, 20.60 mmol) in DMF (25 mL) at 0° C. was added sodium hydride (60% dispersion in mineral oil, 0.82 g, 20.6 mmol) and the mixture was stirred for 30 min. To this was added Intermediate 66 (2.6 g, 10.3 mmol) in portions then the mixture was stirred at RT for 16 h. The mixture was quenched with ice cold water and extracted with EtOAc. The organic layer was dried over Na₂SO₄, filtered and concentrated under reduced pressure. The crude product was purified by flash column chromatography (neutral alumina, eluting 5-10% MeOH in DCM) to provide the title compound (0.65 g). LCMS (Method 1): 1.36 min, 306.2 [M+H]⁺.

### Intermediate 68:7-Amino-8-bromo-2H-pyrido[4,3-b][1,4]oxazin-3 (4H)-one

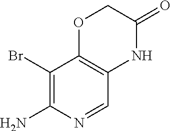 [SMILES: Nc1ncc2c(c1Br)OCC(=O)N2]

To a solution of Intermediate 67 (0.65 g, 2.12 mmol) in acetic acid (10 mL) was added iron powder (0.34 g, 6.09 mmol). The mixture was stirred at 80° C. for 2 h. The mixture was filtered through Celite washing with EtOAc and the filtrate concentrated under reduced pressure to provide the title compound (1.0 g). LCMS (Method 1): 0.55 min, 244.3 [M+H]⁺.

### Intermediate 69:8-Bromo-3,4-dihydro-2H-pyrido[4,3-b][1,4]oxazin-7-amine

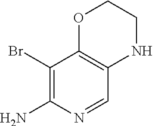 [SMILES: Nc1ncc2c(c1Br)OCCN2]

To a solution of Intermediate 68 (1.0 g, 4.10 mmol) in THF (10 mL) cooled at 0° C. was added borane in THF (1 M, 24.6 mL, 24.6 mmol) then the mixture was stirred at 80° C. for 2 h. The mixture was cooled to 0° C. then quenched by addition of MeOH, which was then heated at 80° C. for 6 h. The mixture was concentrated under reduced pressure. The crude product was purified by preparative HPLC (X SELECT C18, 19×250 mm×5 μm, flow rate: 18 mL/min, 10 mM NH₄HCO₃ in water with MeCN 10% to 30% over 10 min, held at 30% for 1.25 min then ramped to 98% over 0.01 min and held for 0.1 min) to provide the title compound (0.1 g). 1H NMR (400 MHZ; DMSO-d₆) δ: 7.32 (s, 1H), 5.23 (br s, 3H), 4.25 (t, 2H), 3.18 (t, 2H).

### Intermediate 70:2-((3-Bromo-5-nitropyridin-4-yl)amino)-3.3,3-trifluoropropanoic acid

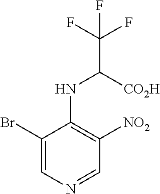 [SMILES: O=C(O)C(Nc1c(Br)cncc1[N+](=O)[O-])C(F)(F)F]

To a solution of 2-amino-3,3,3-trifluoropropanoic acid (0.5 g, 3.49 mmol, CAS 17463-43-3) in acetonitrile (10 mL) was added 3-bromo-4-chloro-5-nitropyridine (0.86 g, 3.67 mmol, CAS 31872-63-6) and DIPEA (1.92 g, 10.5 mmol) and stirred for at RT for 16 h. The mixture was concentrated under reduced pressure. The crude product was purified by flash column chromatography (silica gel, 100-200 mesh eluting 5-10% MeOH in DCM) to provide the title compound (0.70 g). LCMS (Method 1): 1.31 min, 344.0 [M+H]⁺.

### Intermediate 71:8-Bromo-2-(trifluoromethyl)-1,4-dihydropyrido[3.4-b]pyrazin-3 (2H)-one

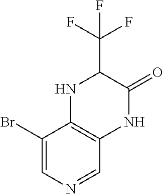 [SMILES: O=C1Nc2cncc(Br)c2NC1C(F)(F)F]

To a solution of Intermediate 70 (0.70 g, 2.04 mmol) in acetic acid (7 mL) was added iron powder (0.34 g, 6.09 mmol). The mixture was stirred at 80° C. for 4 h. The mixture was concentrated under reduced pressure, basified with aqueous sodium bicarbonate and extracted with 10% MeOH in DCM. The combined organic layer was dried over sodium sulfate, filtered and concentrated under reduced pressure. The crude product was purified by flash column chromatography (silica gel, 100-200 mesh eluting 5-10% MeOH in DCM) to provide the title compound (0.45 g). LCMS (Method 1): 0.90 min, 296.0 [M+H]⁺.

### Intermediate 72:8-Bromo-2-(trifluoromethyl)-1,2,3,4-tetrahydropyrido[3.4-b]pyrazine

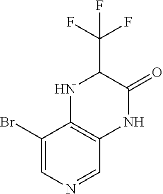 [SMILES: O=C1Nc2cncc(Br)c2NC1C(F)(F)F]

To a solution of Intermediate 71 (0.45 g, 1.52 mmol) in THF (10 mL) cooled at 0° C. was added borane in THF (1 M, 4.57 mL, 4.57 mmol) then the mixture was stirred at RT for 3 h. The mixture was quenched by addition of MeOH, which was then heated at 80° C. for 16 h. The mixture was concentrated under reduced pressure. The crude product was purified by flash column chromatography (silica gel, 100-200 mesh eluting 5-10% MeOH in DCM) to provide the title compound (0.12 g). LCMS (Method 1): 0.95 min, 282.0 [M+H]⁺.

### Intermediate 73:3-((2,4-Dimethoxybenzyl)amino)-1,1,1-trifluoropropan-2-ol

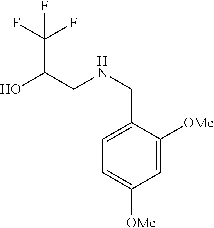 [SMILES: COc1ccc(CNCC(O)C(F)(F)F)c(OC)c1]

To a solution of 3-amino-1, 1, 1-trifluoropropan-2-ol (10 g, 77.5 mmol, CAS 431-38-9) in MeOH (100 mL) was added acetic acid (4.6 mL, 77.5 mmol) followed by 2,4-dimethoxybenzaldehyde (14.2 g, 85.2 mmol) and stirred at RT for 30 min. The mixture was cooled at 0° C. then sodium cyanoborohydride (14.6 g, 232 mmol) was added in portions so the temperature was maintained between 15° C. and 25° C. The mixture was stirred at RT for 16 h. The mixture was concentrated under reduced pressure then quenched by adding water. This was extracted with EtOAc then the organic layer washed with brine, dried over magnesium sulfate, filtered and concentrated under reduced pressure. The crude product was purified by flash column chromatography (neutral alumina, eluting 15-20% EtOAc in petroleum ether) to provide the title compound (11 g). LCMS (Method 1): 1.10 min, 280.2 [M+H]⁺.

### Intermediate 74: tert-Butyl(2,4-dimethoxybenzyl) (3,3,3-trifluoro-2-hydroxypropyl) carbamate

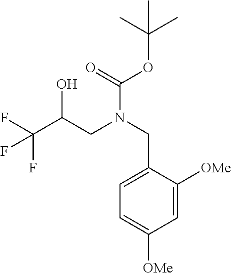 [SMILES: COc1ccc(CN(CC(O)C(F)(F)F)C(=O)OC(C)(C)C)c(OC)c1]

To a stirred solution of Intermediate 73 (11 g, 39.4 mmol) and triethylamine (12.0 mL, 118 mmol) in DCM (10 mL) at 0° C. was added di-tert-butyl dicarbonate (10.3 g, 47.3 mmol) and stirred at RT for 16 h. The mixture was quenched with water then extracted DCM. The organic layer was washed with brine, dried over Na₂SO₄, filtered and concentrated under reduced pressure. The crude product was purified by flash column chromatography (silica gel, 100-200 mesh, eluting 20-25% EtOAc in petroleum ether) to provide the title compound (8.0 g). LCMS (Method 1): 2.17 min, 324.1 [M-tBu+H]⁺.

### Intermediate 75: tert-Butyl(2-((3,5-dibromopyridin-4-yl)oxy)-3,3,3-trifluoropropyl) (2,4-dimethoxybenzyl) carbamate

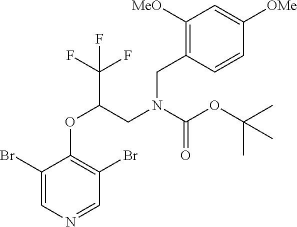 [SMILES: COc1ccc(CN(CC(Oc2c(Br)cncc2Br)C(F)(F)F)C(=O)OC(C)(C)C)c(OC)c1]

To a suspension of Intermediate 74 (8.0 g, 21.1 mmol) Cs₂CO₃ (20.6 g, 63.3 mmol) in DMF (80 mL) at 0° C. was added 3,5-dibromo-4-chloropyridine (5.72 g, 21.1 mmol, CAS 13626-17-0) and the mixture was stirred at RT for 16 h. The mixture was quenched with water and extracted with EtOAc. The organic layer was washed with brine, dried over magnesium sulfate, filtered and concentrated under reduced pressure. The crude product was purified by flash column chromatography (silica gel, 100-200 mesh, eluting 10-15% EtOAc in petroleum ether) to provide the title compound (11 g). LCMS (Method 1): 2.65 min, 615.2 [M+H]⁺.

### Intermediate 76:2-((3,5-Dibromopyridin-4-yl)oxy)-N-(2.4-dimethoxybenzyl)-3,3,3-trifluoropropan-1-amine hydrochloride

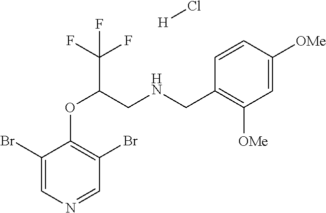

To a stirred solution of Intermediate 75 (4.0 g, 6.5 mmol) in EtOAc (20 mL) at 0° C. was added 4 M HCl in 1,4-dioxane (40 mL) and stirred at RT for 3 h. The mixture was concentrated under reduced pressure to provide the title compound (3.5 g). LCMS (Method 1): 2.20 min, 515.3 [M+H]⁺.

### Intermediate 77:8-Bromo-4-(2,4-dimethoxybenzyl)-2-(trifluoromethyl)-3,4-dihydro-2H-pyrido[4,3-b] [1,4]oxazine

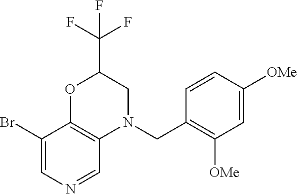 [SMILES: COc1ccc(CN2CC(C(F)(F)F)Oc3c(Br)cncc32)c(OC)c1]

To a solution of Intermediate 76 (1.7 g, 3.08 mmol) in toluene (12.5 mL) was added sodium t-butoxide (2.01 g, 20.9 mmol) and the mixture was degassed with nitrogen for 5 min. Pd₂(dba)₃ (0.28 g, 0.31 mmol) and BINAP (0.39 g, 0.62 mmol) were added and the mixture stirred at 110° C. for 16 h in a sealed tube. The mixture was allowed to cool then diluted water and extracted with EtOAc. The organic layer was dried over sodium sulfate, filtered and concentrated under reduced pressure. The crude product was purified by flash column chromatography (silica gel, 100-200 mesh, eluting 20-25% EtOAc in petroleum ether) to provide the title compound (0.30 g). LCMS (Method 1): 2.06 min, 433.2 [M+H]⁺.

### Intermediate 78: 8-Bromo-2-(trifluoromethyl)-3,4-dihydro-2H-pyrido[4,3-b][1 4]oxazine

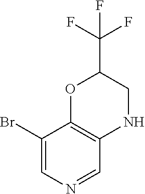 [SMILES: FC(F)(F)C1CNc2cncc(Br)c2O1]

To a stirred solution of Intermediate 77 (0.30 g, 0.69 mmol) in EtOAc (1.5 mL) at 0° C. was added 4 M HCl in 1,4-dioxane (3 mL) and this was then stirred at 60° C. for 2 h. The mixture was concentrated under reduced pressure then the residue was basified with saturated aqueous sodium bicarbonate and extracted with EtOAc. The organic layer was washed with brine, dried over magnesium sulfate, filtered and concentrated under reduced pressure. The crude product was purified by flash column chromatography (silica gel, 100-200 mesh, eluting 35-40% EtOAc in petroleum ether) to provide the title compound (0.19 g). LCMS (Method 1): 1.39 min, 283.1 [M+H]⁺.

### Intermediate 79:3.5-Dibromo-2-methylpyridin-4-ol hydrobromide

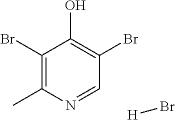

To a stirred solution of 2-methylpyridin-4-ol (20 g, 183 mmol, CAS 18615-86-6) in hydrobromic acid (48% aqueous, 70 mL) at 0° C. was added a solution of bromine (18.6 mL, 367 mmol) in acetic acid (80 mL) dropwise. The mixture was then stirred at RT for 3 h. The mixture was poured onto ice water and the solid was filtered washing with cold water. The solid was dried under reduced pressure to provide the title compound (22 g). 1H NMR (400 MHz; DMSO-d₆) δ: 12.31 (br s, 1H), 8.19 (s, 1H), 3.57 (br s, 1H), 2.39 (s, 3H).

### Intermediate 80:3,5-Dibromo-4-chloro-2-methylpyridine

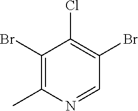

To a stirred suspension of Intermediate 79 (17 g, 48.9 mmol) in DCM (170 mL) at 0° C. was added thionyl chloride (13.9 mL, 191 mmol) and DMF (1.7 mL). The mixture was then stirred at 60° C. for 4 h. The mixture was allowed to cool then poured into cold saturated aqueous sodium bicarbonate. This was extracted with DCM and the organic layer dried over sodium sulfate, filtered and concentrated under reduced pressure to provide the title compound (18 g). LCMS (Method 1): 2.19 min, 285.9 [M+H]⁺.

### Intermediate 81:2-((3,5-Dibromo-2-methylpyridin-4-yl)oxy)-N-(2,4-dimethoxybenzyl) ethan-1-amine

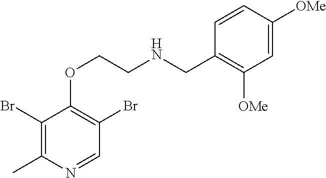 [SMILES: COc1ccc(CNCCOc2c(Br)cnc(C)c2Br)c(OC)c1]

To a stirred solution of 2-((2,4-dimethoxybenzyl)amino) ethan-1-ol (9.98 g, 47.3 mmol, CAS 40171-89-9) in DMF (100 mL) at 0° C. was added sodium hydride (60% dispersion in mineral oil, 2.52 g, 62.1 mmol) and the mixture was stirred for 30 min. To this was added Intermediate 80 (9.0 g, 31.5 mmol) then the mixture was stirred at RT for 16 h. The mixture was quenched with ice cold water and extracted with EtOAc. The organic layer was dried over Na₂SO₄, filtered and concentrated under reduced pressure. The crude product was purified by flash column chromatography (neutral alumina, eluting 50% EtOAc in petroleum ether) to provide the title compound (3.6 g). LCMS (Method 1): 1.40 min, 461.3 [M+H]⁺.

### Intermediate 82:8-Bromo-4-(2,4-dimethoxybenzyl)-7-methyl-3,4-dihydro-2H-pyrido[4,3-b][1,4]oxazine

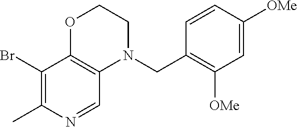 [SMILES: COc1ccc(CN2CCOc3c2cnc(C)c3Br)c(OC)c1]

To a solution of Intermediate 81 (2.5 g, 5.43 mmol) in toluene (25 mL) was added sodium t-butoxide (1.56 g, 16.3 mmol) and the mixture was degassed with nitrogen for 5 min. Pd₂(dba)₃ (0.50 g, 0.54 mmol) and X-Phos (0.39 g, 0.62 mmol) were added and the mixture stirred at 100° C. for 4 h in a sealed tube. The mixture was allowed to cool then filtered through Celite and washed with EtOAc. The filtrate was washed with water, brine and concentrated under reduced pressure. The crude product was purified by flash column chromatography (silica gel, 230-400 mesh, eluting 15% EtOAc in petroleum ether) to provide the title compound (0.28 g). LCMS (Method 1): 1.50 min, 379.2 [M+H]⁺.

### Intermediate 83: 8-Bromo-7-methyl-3,4-dihydro-2H-pyrido[4,3-b][1 4]oxazine hydrochloride

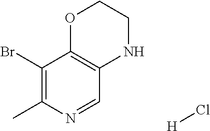

To a stirred solution of Intermediate 82 (0.28 g, 0.74 mmol) in EtOAc (3 mL) at 0° C. was added 4 M HCl in 1,4-dioxane (3 mL) and stirred at 80° C. for 6 h. The mixture was concentrated under reduced pressure and the crude product was triturated with diethyl ether and the solid filtered to provide the title compound (0.15 g). LCMS (Method 1): 0.98 min, 229.0 [M+H]⁺.

### Intermediate 84: tert-Butyl(2-((3,5-dibromo-2-methylpyridin-4-yl)oxy)-3,3,3-trifluoropropyl) (2,4-dimethoxybenzyl) carbamate

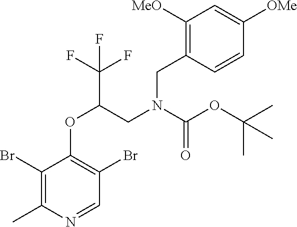 [SMILES: COc1ccc(CN(CC(Oc2c(Br)cnc(C)c2Br)C(F)(F)F)C(=O)OC(C)(C)C)c(OC)c1]

To a suspension of Intermediate 74 (4.5 g, 11.9 mmol) Cs₂CO₃ (11.6 g, 35.7 mmol) in DMF (45 mL) at 0° C. was added 3,5-dibromo-4-chloro-2-methylpyridine (3.37 g, 11.9 mmol, CAS 1188024 Jan. 2) and the mixture was stirred at RT for 16 h. The mixture was quenched with water and extracted with EtOAc. The organic layer was washed with brine, dried over magnesium sulfate, filtered and concentrated under reduced pressure. The crude product was purified by flash column chromatography (silica gel, 100-200 mesh, eluting 30% EtOAc in petroleum ether) to provide the title compound (5.1 g). LCMS (Method 1): 2.71 min, 629.2 [M+H]⁺.

### Intermediate 85:2-((3,5-Dibromo-2-methylpyridin-4-yl)oxy)-N-(2,4-dimethoxybenzyl)-3,3,3-trifluoropropan-1-amine hydrochloride

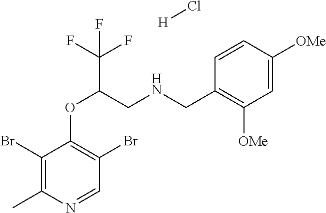

To a stirred solution of Intermediate 84 (3.5 g, 5.57 mmol) in EtOAc (25 mL) at 0° C. was added 4 M HCl in 1,4-dioxane (50 mL) and stirred at RT for 3 h. The mixture was concentrated under reduced pressure to provide the title compound (2.7 g). LCMS (Method 1): 2.24 min, 529.2 [M+H]⁺.

### Intermediate 86:8-Bromo-4-(2,4-dimethoxybenzyl)-7-methyl-2-(trifluoromethyl)-3,4-dihydro-2H-pyrido[4,3-b] [1,4]oxazine

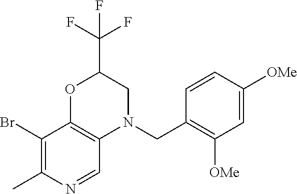 [SMILES: COc1ccc(CN2CC(C(F)(F)F)Oc3c2cnc(C)c3Br)c(OC)c1]

To a solution of Intermediate 85 (2.7 g, 4.78 mmol) in toluene (27 mL) was added sodium t-butoxide (1.37 g, 14.3 mmol) and the mixture was degassed with nitrogen for 5 min. Pd₂(dba)₃ (0.44 g, 0.48 mmol) and BINAP (0.59 g, 0.95 mmol) were added and the mixture stirred at 110° C. for 5 h in a sealed tube. The mixture was allowed to cool then diluted water and extracted with EtOAc. The organic layer was dried over sodium sulfate, filtered and concentrated under reduced pressure. The crude product was purified by flash column chromatography (silica gel, 100-200 mesh, eluting 20% EtOAc in petroleum ether) to provide the title compound (0.60 g). LCMS (Method 16): 3.35 min, 448.7 [M+H]⁺.

### Intermediate 87:8-Bromo-7-methyl-2-(trifluoromethyl)-3.4-dihydro-2H-pyrido[4,3-b][1,4]oxazine hydrochloride

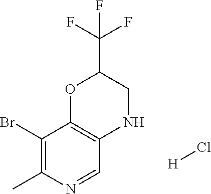 [SMILES: Cc1ncc2c(c1Br)OC(C(F)(F)F)CN2 ; Cl]

To a stirred solution of Intermediate 86 (0.60 g, 1.34 mmol) in EtOAc (3 mL) at 0° C. was added 4 M HCl in 1,4-dioxane (6 mL) and this was then stirred at 80° C. for 2 h.

The mixture was concentrated under reduced pressure then the solid residue was triturated with diethyl ether and filtered to provide the title compound (0.42 g). LCMS (Method 1): 1.26 min, 297.2 [M+H]⁺.

### Intermediate 88:8-Bromo-2-ethyl-2H-pyrido[4,3-b] [1,4]oxazin-3 (4H)-one

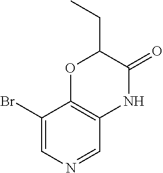

To a solution of 3-amino-5-bromopyridin-4-ol (3.42 g, 17.6 mmol, CAS 13073-25-1) in MeCN (35 mL) was added potassium carbonate (7.28 g, 52.7 mmol) at 0° C. then the mixture was stirred at RT for 30 min. 2-Bromo-butanoyl bromide (2.55 mL, 21.1 mmol) was added dropwise at 0° C. then the mixture was stirred at RT for 30 min before heating to 80° C. for 16 h. The mixture was concentrated under reduced pressure then the crude product was purified by flash column chromatography (silica gel, 100-200 mesh, eluting 0-100% EtOAc in petroleum ether) to provide the title compound (1.32 g). 1H NMR (400 MHZ; DMSO-d₆) δ: 11.06 (br s, 1H), 8.26 (s, 1H), 8.02 (s, 1H), 4.87 (dd, 1H), 1.88-1.78 (m, 2H), 1.00 (t, 3H).

### Intermediate 89:8-Bromo-2-ethyl-3,4-dihydro-2H-pyrido[4,3-b][1,4]oxazine

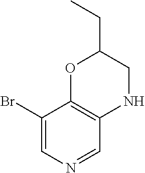

To a solution of Intermediate 88 (1.31 g, 4.38 mmol) in THF (13 mL) cooled at 0° C. was added borane in THF (1 M, 21.9 mL, 21.9 mmol) then the mixture was allowed to RT for 2 h. The mixture was quenched by addition of MeOH at 0° C. then heated at 60° C. for 16 h. The mixture was concentrated under reduced pressure. The residue was partitioned with water and EtOAc, organic layer washed with brine, dried over Na₂SO₄, filtered and concentrated under reduced pressure. The crude was purified by flash column chromatography (silica gel, 100-200 mesh, eluting 0-100% EtOAc in petroleum ether) to provide the title compound (0.50 g). LCMS (Method 1): 1.12 min, 243.1 [M+H]⁺.

### Intermediate 90: Methyl 2-((3-bromo-5-nitropyridin-4-yl)oxy)-3-methylbutanoate

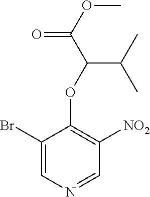 [SMILES: COC(=O)C(Oc1c(Br)cncc1[N+](=O)[O-])C(C)C]

To a stirred solution of methyl 2-hydroxy-3-methylbutanoate (1.31 g, 12.6 mmol, CAS 17417-00-4) and 3-bromo-4-chloro-5-nitropyridine (2.0 g, 8.42 mmol, CAS 31872-63-6) in DMF (25 mL) was added Cs₂CO₃ (11.0 g, 33.7 mmol) was added and the mixture was stirred at RT for 2 h. The mixture was diluted with water and extracted with EtOAc. The organic layer was washed with brine, dried over Na₂SO₄, filtered and concentrated under reduced pressure. The crude product was purified by flash column chromatography (40 g silica gel, eluting 2-5% EtOAc in petroleum ether) to provide the title compound (1.8 g). LCMS (Method 17): 3.13 min, 333.1 [M+H]⁺.

### Intermediate 91:8-Bromo-2-isopropyl-2H-pyrido[4,3-b][1,4]oxazin-3 (4H)-one

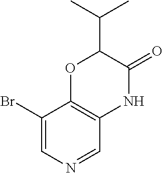

To a solution of Intermediate 90 (1.75 g, 5.25 mmol) in acetic acid (17.5 mL) was added iron powder (1.5 g, 26.7 mmol). The mixture was stirred at 80° C. for 2 h. The mixture was basified carefully with aqueous sodium bicarbonate then extracted with DCM. The organic layer was dried over sodium sulfate, filtered and concentrated under reduced pressure to provide the title compound (1.0 g). LCMS (Method 1): 1.54 min, 271.1 [M+H]⁺.

### Intermediate 92:8-Bromo-2-isopropyl-3,4-dihydro-2H-pyrido[4,3-b][1,4]oxazine

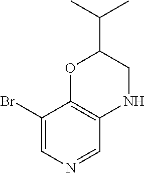

To a solution of Intermediate 91 (1.0 g, 2.58 mmol) in THF (10 mL) cooled at 0° C. was added borane in THF (1 M, 5 mL, 5.0 mmol) then the mixture was allowed to RT over 2 h. The mixture was quenched by addition of MeOH (5 mL) at 0° C. then heated at 60° C. for 16 h. The mixture was concentrated under reduced pressure. The residue was partitioned with water and EtOAc, organic layer washed with brine, dried over Na₂SO₄, filtered and concentrated under reduced pressure. The crude was purified by flash column chromatography (silica gel, 100-200 mesh, eluting 2-5% MeOH in DCM) to provide the title compound (0.65 g). LCMS (Method 1): 1.25 min, 257.1 [M+H]⁺.

### Intermediate 93: Ethyl 2-((3-bromo-5-nitropyridin-4-yl)oxy)-2-cyclopropylacetate

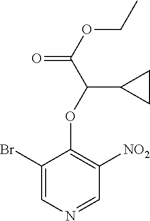 [SMILES: CCOC(=O)C(Oc1c(Br)cncc1[N+](=O)[O-])C1CC1]

To a stirred solution of ethyl 2-cyclopropyl-2-hydroxyacetate (1.56 g, 7.58 mmol, CAS 1185387-66-9) and 3-bromo-4-chloro-5-nitropyridine (1.5 g, 6.32 mmol, CAS 31872-63-6) in DMF (20 mL) was added Cs₂CO₃ (1.03 g, 3.16 mmol) and the mixture was stirred at RT for 16 h. The mixture was diluted with water and extracted with EtOAc. The organic layer was washed with brine, dried over Na₂SO₄, filtered and concentrated under reduced pressure. The crude product was purified by flash column chromatography (40 g silica gel, eluting 15% EtOAc in iso-hexane) to provide the title compound (0.4 g). LCMS (Method 1): 1.92 min, 345.2 [M+H]⁺.

### Intermediate 94:8-Bromo-2-cyclopropyl-2H-pyrido[4,3-b][1,4]oxazin-3 (4H)-one

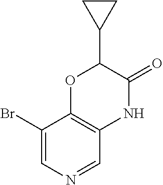 [SMILES: O=C1Nc2cncc(Br)c2OC1C1CC1]

To a solution of Intermediate 93 (0.4 g, 0.75 mmol) in acetic acid (4 mL) was added iron powder (0.21 g, 3.77 mmol). The mixture was stirred at 80° C. for 3 h. The mixture was concentrated under reduced pressure, dissolved in EtOAc and basified carefully with aqueous sodium bicarbonate. The organic layer was washed with water, brine, dried over sodium sulfate, filtered and concentrated under reduced pressure to provide the title compound (0.2 g). LCMS (Method 1): 1.38 min, 269.1 [M+H]⁺.

### Intermediate 95:8-Bromo-2-cyclopropyl-3,4-dihydro-2H-pyrido[4,3-b][1,4]oxazine

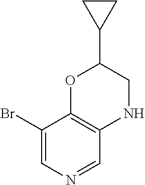 [SMILES: Brc1cncc2c1OC(C1CC1)CN2]

To a solution of Intermediate 94 (0.2 g, 0.56 mmol) in THF (3 mL) cooled at 0° C. was added borane in THF (1 M, 0.56 mL, 0.56 mmol) then the mixture was allowed to RT for 2 h. The mixture was quenched by addition of MeOH at 0° C. then heated at 60° C. for 16 h. The mixture was concentrated under reduced pressure. The crude was purified by automated flash chromatography (10 Biotage Snap neutral alumina, eluting 50% EtOAc in isohexane) to provide the title compound (60 mg). LCMS (Method 1): 1.19 min, 255.1 [M+H]⁺.

### Intermediate 96:8-Bromo-7-methyl-2-(trifluoromethyl)-3.4-dihydro-2H-pyrido[4,3-b][1.4]oxazine

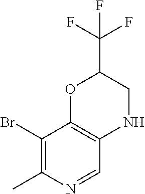 [SMILES: Cc1ncc2c(c1Br)OC(C(F)(F)F)CN2]

To a stirred solution of Intermediate 86 (0.49 g, 1.03 mmol) in EtOAc (3 mL) at 0° C. was added 4 M HCl in 1,4-dioxane (5 mL) and this was then stirred at 60° C. for 3 h. The mixture was concentrated under reduced pressure then the residue was basified with saturated aqueous sodium bicarbonate and extracted with EtOAc. The organic layer was washed with brine, dried over magnesium sulfate, filtered and concentrated under reduced pressure. The crude product was purified by flash column chromatography (silica gel, 100-200 mesh, eluting 0-70% EtOAc in petroleum ether) to provide the title compound (0.21 g). LCMS (Method 1): 1.22 min, 299.1 [M+H]⁺.

### Intermediate 97: 7-Methyl-2-(trifluoromethyl)-3,4-dihydro-2H-pyrido[4,3-b][1 4]oxazine-8-carbonitrile

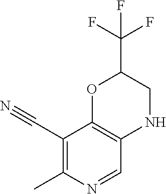 [SMILES: Cc1ncc2c(c1C#N)OC(C(F)(F)F)CN2]

To a solution of Intermediate 96 (0.17 g, 0.44 mmol) in 1,4-dioxane (0.5 mL) was added zinc (II) cyanide (0.10 g, 0.88 mmol) then the mixture was purged with nitrogen gas for 10 min. Cs₂CO₃ (0.43 g, 1.32 mmol) and Pd(dppf)Cl₂ (32 mg, 0.04 mmol) were added and the mixture was stirred at 130° C. for 16 h in a sealed vessel. The mixture was diluted with water and extracted into EtOAc. The organic layer was washed with water, brine, dried over sodium sulfate, filtered and concentrated under reduced pressure. The crude product was purified by flash column chromatography (silica gel, 100-200 mesh, eluting 0-40% EtOAc in petroleum ether) to provide the title compound (0.12 g). LCMS (Method 1): 1.36 min, 244.1 [M+H]⁺.

### Intermediate 98:4-(2,4-Dimethoxybenzyl)-2-(trifluoromethyl)-3,4-dihydro-2H-pyrido[4,3-b][1,4]oxazine-8-carbonitrile

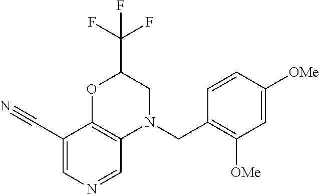 [SMILES: COc1ccc(CN2CC(C(F)(F)F)Oc3c(C#N)cncc32)c(OC)c1]

A mixture of Intermediate 77 (0.50 g, 0.84 mmol), sodium carbonate (89 mg, 0.84 mmol) and potassium hexacyanoferrate (II) (71 mg, 0.17 mmol) in DMF (5 mL) was degassed with argon for 15 min. To this was added palladium acetate (19 mg, 0.08 mmol) and the mixture was stirred at 125° C. for 16 h in a sealed vessel. The mixture was diluted with water and extracted into EtOAc. The organic layer was washed with water, brine, dried over sodium sulfate, filtered and concentrated under reduced pressure. The crude product was purified by flash column chromatography (silica gel, 100-200 mesh, eluting 0-100% EtOAc in petroleum ether) to provide the title compound (0.17 g). LCMS (Method 1): 1.98 min, 380.2 [M+H]⁺.

### Intermediate 99:2-(Trifluoromethyl)-3,4-dihydro-2H-pyrido[4,3-b][1,4]oxazine-8-carbonitrile

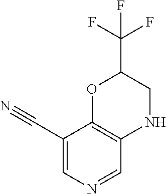 [SMILES: N#Cc1cncc2c1OC(C(F)(F)F)CN2]

To a stirred solution of Intermediate 98 (0.21 g, 0.53 mmol) in EtOAc (1 mL) at 0° C. was added 4 M HCl in 1,4-dioxane (2 mL) and this was then stirred at 60° C. for 3 h. The mixture was concentrated under reduced pressure then the residue was basified with saturated aqueous sodium bicarbonate and extracted with EtOAc. The organic layer was washed with brine, dried over sodium sulfate, filtered and concentrated under reduced pressure. The crude product was purified by flash column chromatography (silica gel, 100-200 mesh, eluting 30-40% EtOAc in petroleum ether) to provide the title compound (0.21 g). LCMS (Method 1): 1.35 min, 230.1 [M+H]⁺.

### Intermediate 100:4-(2,4-Dimethoxybenzyl)-8-methoxy-2-(trifluoromethyl)-3,4-dihydro-2H-pyrido[4,3-b][1,4]oxazine

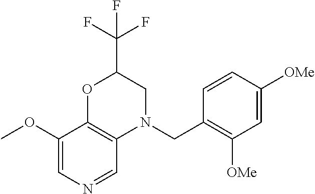 [SMILES: COc1ccc(CN2CC(C(F)(F)F)Oc3c(OC)cncc32)c(OC)c1]

A solution of Intermediate 77 (1.40 g, 2.20 mmol) in toluene (15 mL) was degassed with argon for 10 min. tBuBrettPhos Pd G3 (0.02 g, 0.02 mmol) and sodium methoxide (30% in MeOH, 0.47 g, 8.79 mmol) were added and the mixture was stirred at 80° C. for 16 h. The mixture was diluted with water and extracted into EtOAc. The organic layer was washed with water, brine, dried over sodium sulfate, filtered and concentrated under reduced pressure. The crude product was purified by flash column chromatography (silica gel, 100-200 mesh, eluting 0-30% EtOAc in petroleum ether) to provide the title compound (0.38 g). LCMS (Method 1): 1.50 min, 385.3 [M+H]⁺.

### Intermediate 101:8-Methoxy-2-(trifluoromethyl)-3,4-dihydro-2H-pyrido[4,3-b][1,4]oxazine

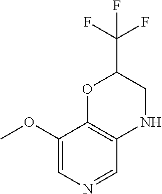 [SMILES: COc1cncc2c1OC(C(F)(F)F)CN2]

To a stirred solution of Intermediate 100 (0.35 g, 0.88 mmol) in EtOAc (1.8 mL) at 0° C. was added 4 M HCl in 1,4-dioxane (0.88 mL) and this was then stirred at 60° C. for 3 h. The mixture was concentrated under reduced pressure then the residue was basified with saturated aqueous sodium bicarbonate and extracted with EtOAc. The organic layer was washed with brine, dried over sodium sulfate, filtered and concentrated under reduced pressure. The crude product was purified by flash column chromatography (silica gel, 100-200 mesh, eluting 0-35% EtOAc in petroleum ether) to provide the title compound (0.20 g). LCMS (Method 1): 0.99 min, 235.1 [M+H]⁺.

### Intermediate 102: Ethyl 3-(3-bromo-5-nitropyridin-4-yl)-2-cyanopropanoate

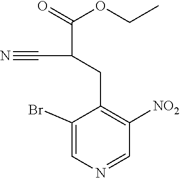 [SMILES: CCOC(=O)C(C#N)Cc1c(Br)cncc1[N+](=O)[O-]]

To a solution of ethyl 2-cyanoacetate (3.1 mL, 29.1 mmol, CAS 105-56-6) in THF (50 mL) was added LDA (2 M in THF, 19.4 mL, 38.8 mmol) at 0° C. and the mixture was stirred for 30 min. A solution of 3-bromo-4-(bromomethyl)-5-nitropyridine (7.0 g, 19.4 mmol, CAS 1807024-35-6) in THF (20 mL) were added at 0° C. then the mixture was stirred at RT for 2 h. The mixture was diluted with saturated aqueous ammonium chloride and extracted into EtOAc. The organic layer was washed with water, brine, dried over sodium sulfate, filtered and concentrated under reduced pressure. The crude product was purified by flash column chromatography (silica gel, 100-200 mesh, eluting 0-10% EtOAc in petroleum ether) to provide the title compound (4.8 g). LCMS (Method 1): 1.81 min, 330.2 [M+H]⁺.

### Intermediate 103:5-Bromo-2-oxo-1,2,3,4-tetrahydro-1,7-naphthyridine-3-carbonitrile

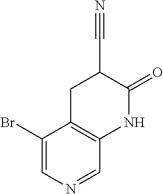 [SMILES: N#CC1Cc2c(Br)cncc2NC1=O]

To a solution of Intermediate 102 (1.8 g, 4.28 mmol) in ethanol (30 mL) and water (12 mL) was added iron powder (1.21 g, 21.7 mmol) and ammonium chloride (1.16 g, 21.7 mmol) and the mixture was stirred at 80° C. for 6 h. The mixture was filtered through Celite and washed with MeOH. The filtrate was concentrated under reduced pressure, dissolved in EtOAc and washed with water, brine, dried over sodium sulfate, filtered and concentrated under reduced pressure to provide the title compound (2.1 g). LCMS (Method 1): 1.25 min, 254.1 [M+H]⁺.

### Intermediate 104:5-Bromo-1,2,3,4-tetrahydro-1,7-naphthyridine-3-carbonitrile

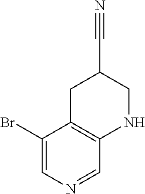 [SMILES: N#CC1CNc2cncc(Br)c2C1]

To a solution of Intermediate 103 (1.6 g, 2.22 mmol) in THF (10 mL) cooled at 0° C. was added borane dimethylsulfide complex (2 M in THF, 2.78 mL, 5.56 mmol) then the mixture was allowed to RT for 2 h. The mixture was quenched by addition of MeOH at 0° C. then heated at 60° C. for 16 h. The mixture was concentrated under reduced pressure then dissolved in DCM and washed with water, brine, dried over sodium sulfate, filtered and concentrated under reduced pressure. The crude was purified by preparative HPLC (X-Select C18, 19×250 mm×5 μm, flow rate: 18 mL/min, 10 mM NH₄HCO₃ in water with MeCN 30% to 38% over 7 min, ramped to 100% over 0.5 min and held for 3.5 min) to provide the title compound (1.2 g). LCMS (Method 1): 0.95 min, 240.0 [M+H]⁺.

### Intermediate 105:5-Bromo-N-cyclopropyl-3-(difluoromethyl)-N-methylpicolinamide

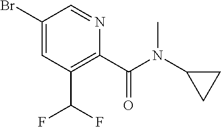 [SMILES: CN(C(=O)c1ncc(Br)cc1C(F)F)C1CC1]

To a solution of 5-bromo-3-(difluoromethyl)pyridine-2-carboxylic acid (0.50 g, 1.98 mmol, CAS 1404337-62-7) in THF (5 mL) cooled at 0° C. was added DIPEA (1.72 mL, 9.92 mmol) then after 5 min, T3P (1.79 mL, 5.95 mmol) and N-methylcyclopropylamine (0.21 g, 2.98 mmol, CAS 5163-20-2) were added and the mixture was stirred at RT for 3 h. The mixture was poured into water and extracted with EtOAc. The organics were dried over Na₂SO₄, filtered and concentrated under reduced pressure. The crude was purified by flash column chromatography (silica gel, 0-100% EtOAc in hexane) to provide the title compound (0.35 g). LCMS (Method 4): 1.80 min, 305.3 [M+H]⁺.

### Intermediate 106: N-Cyclopropyl-3-(difluoromethyl)-5-((diphenylmethylene)amino)-N-methylpicolinamide

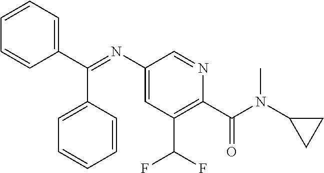 [SMILES: CN(C(=O)c1ncc(N=C(c2ccccc2)c2ccccc2)cc1C(F)F)C1CC1]

To a solution of Intermediate 105 (0.50 g, 1.49 mmol) in toluene (10 mL) was added Cs₂CO₃ (1.46 g, 4.47 mmol) and the mixture was degassed with argon for 20 min. Pd₂(dba)₃ (0.14 g, 0.15 mmol) and X-Phos (0.14 g, 0.30 mmol) were added and the mixture stirred at 110° C. for 16 h. The mixture was allowed to cool diluted with water and extracted with EtOAc. The organics were washed with water, brine, dried over Na₂SO₄, filtered and concentrated under reduced pressure. The crude product was purified by flash column chromatography (silica gel, 230-400 mesh, eluting 0-100% EtOAc in petroleum ether) to provide the title compound (0.50 g). LCMS (Method 4): 2.22 min, 406.4 [M+H]⁺.

### Intermediate 107:5-Amino-N-cyclopropyl-3-(difluoromethyl)-N-methylpicolinamide

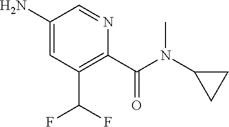 [SMILES: CN(C(=O)c1ncc(N)cc1C(F)F)C1CC1]

To a solution of Intermediate 106 (0.70 g, 1.57 mmol) in DCM (10 mL) at 0° C. was added TFA (0.36 mL, 4.71 mmol) and the mixture was stirred at RT for 3 h. The mixture poured into water and extracted with DCM. The organics were dried over Na₂SO₄, filtered and concentrated under reduced pressure. The crude product was purified by flash column chromatography (silica gel, 230-400 mesh, eluting 0-100% EtOAc in petroleum ether) to provide the title compound (0.20 g). LCMS (Method 19): 2.34 min, 242.0 [M+H]⁺.

### SYNTHESIS OF EXAMPLES

### Example 1:5-Bromo-N-(5-chloro-6-(2H-1,2,3-triazol-2-yl)pyridin-3-yl)-3,4-dihydroquinoline-1 (2H)-carboxamide

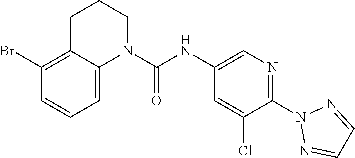 [SMILES: O=C(Nc1cnc(-n2nccn2)c(Cl)c1)N1CCCc2c(Br)cccc21]

To a stirred solution of 5-bromo-1,2,3,4-tetrahydroquinoline (0.25 g, 1.17 mmol, CAS 114744-50-2) in DCM (10 mL) at 0° C. was added 5-chloro-6-(2H-1,2,3-triazol-2-yl)pyridin-3-amine (0.25 g, 1.28 mmol, CAS 1832583-43-3) and triphosgene (0.24 g, 0.82 mmol) and stirred at 0° C. for 15 min. Triethylamine (0.33 mL, 2.35 mmol) was added dropwise then the mixture was stirred at RT for 3 h. The mixture was diluted with DCM and washed with saturated aqueous sodium bicarbonate, water, dried over sodium sulfate, filtered and concentrated under reduced pressure. The crude product was purified by preparative HPLC (Sunfire C18, 19×150 mm×5 μm, flow rate: 20 mL/min, 0.1% TFA in water with MeCN 10% to 60% over 9 min, held at 60% for 5 min then ramped to 100% over 0.01 min and held for 1.9 min) to provide the title compound (39 mg). 1H NMR (400 MHz; DMSO-d₆) δ: 9.69 (br s, 1H), 8.61 (d, 1H), 8.36 (d, 1H), 8.15 (s, 2H), 7.44 (dd, 1H), 7.38 (dd, 1H), 7.13 (t, 1H), 3.76 (dd, 2H), 2.78 (t, 2H), 1.94-2.00 (m, 2H). LCMS (Method 5): 2.22 min, 431.1 [M−H]⁻.

### Example 2: N-(6-(2H-1,2,3-Triazol-2-yl)-5-(trifluoromethyl)pyridin-3-yl)-5-bromo-3,4-dihydroquinoline-1 (2H)-carboxamide

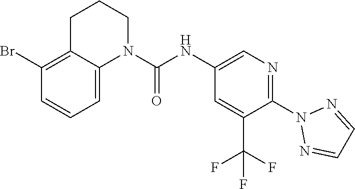 [SMILES: O=C(Nc1cnc(-n2nccn2)c(C(F)(F)F)c1)N1CCCc2c(Br)cccc21]

To a stirred solution of 5-bromo-1,2,3,4-tetrahydroquinoline (0.25 g, 1.17 mmol, CAS 114744-50-2) in DCM (10 mL) at 0° C. was added 6-(2H-1,2,3-triazol-2-yl)-5-(trifluoromethyl)pyridin-3-amine (0.30 g, 1.29 mmol, CAS 1832582-59-8) and triphosgene (0.24 g, 0.82 mmol) and stirred at 0° C. for 15 min. Triethylamine (0.33 mL, 2.35 mmol) was added dropwise then the mixture was stirred at RT for 3 h. The mixture was diluted with DCM and washed with saturated aqueous sodium bicarbonate, water, dried over sodium sulfate, filtered and concentrated under reduced pressure. The crude product was purified by preparative HPLC (X SELECT C18, 19×250 mm×5 μm, flow rate: 18 mL/min, 0.1% TFA in water with MeCN 30% to 80% over 9 min, held at 80% for 1 min then ramped to 100% over 0.1 min and held for 2.9 min) to provide the title compound (57 mg). 1H NMR (400 MHz; DMSO-d₆) δ: 9.82 (br s, 1H), 8.92 (d, 1H), 8.60 (d, 1H), 8.16 (s, 2H), 7.47 (dd, 1H), 7.40 (dd, 1H), 7.13 (t, 1H), 3.77 (dd, 2H), 2.79 (t, 2H), 1.95-2.01 (m, 2H). LCMS (Method 5): 2.29 min, 465.1 [M−H]⁻.

### Example 3: N-(5-Chloro-6-(2H-1,2,3-triazol-2-yl)pyridin-3-yl)-5-methoxy-3,4-dihydroquinoline-1 (2H)-carboxamide

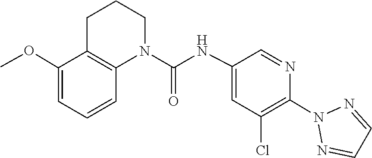 [SMILES: COc1cccc2c1CCCN2C(=O)Nc1cnc(-n2nccn2)c(Cl)c1]

To a stirred solution of 5-chloro-6-(2H-1,2,3-triazol-2-yl)pyridin-3-amine (0.18 g, 0.92 mmol, CAS 1832583-43-3) in DCM (2 mL) at 0° C. was added triethylamine (0.25 mL, 1.84 mmol) and triphosgene (0.19 g, 0.64 mmol) and stirred at 0° C. for 30 min. 5-Methoxy-1,2,3,4-tetrahydroquinoline (0.25 g, 1.17 mmol, CAS 30389-37-8) was added then the mixture was stirred at RT for 16 h. The mixture was diluted with DCM and washed with saturated aqueous sodium bicarbonate, water, dried over sodium sulfate, filtered and concentrated under reduced pressure. The crude product was purified by preparative HPLC (X TERRA C18, 19×250 mm×10 μm, flow rate: 18 mL/min, 0.1% TFA in water with MeCN 20% to 70% over 9 min, held at 70% for 5 min then ramped to 100% over 0.1 min and held for 1.9 min) to provide the title compound (26 mg). 1H NMR (400 MHZ; DMSO-d₆) δ: 9.60 (br s, 1H), 8.62 (d, 1H), 8.36 (d, 1H), 8.14 (s, 2H), 7.15 (t, 1H), 7.02 (d, 1H), 6.74 (d, 1H), 3.81 (s, 3H), 3.72 (dd, 2H), 2.66 (t, 2H), 1.88-1.95 (m, 2H). LCMS (Method 6): 3.45 min, 385.3 [M+H]⁺.

### Example 4: N-(6-(2H-1,2,3-Triazol-2-yl)-5-(trifluoromethyl)pyridin-3-yl)-8-bromo-2,3-dihydro-4H-benzo[b][1,4]oxazine-4-carboxamide

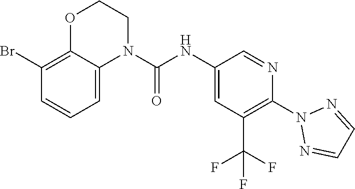 [SMILES: O=C(Nc1cnc(-n2nccn2)c(C(F)(F)F)c1)N1CCOc2c(Br)cccc21]

To a stirred solution of 8-bromo-3,4-dihydro-2H-benzo[b][1,4]oxazine (0.25 g, 1.16 mmol, CAS 625394-65-2) in DCM (2 mL) at 0° C. was added triphosgene (0.34 g, 1.16 mmol) and triethylamine (0.32 mL, 2.32 mmol) stirred at 0° C. for 15 min. Then 6-(2H-1,2,3-triazol-2-yl)-5-(trifluoromethyl)pyridin-3-amine (0.26 g, 1.16 mmol, CAS 1832582-59-8) was added and the mixture was stirred at RT for 16 h. The mixture was diluted with saturated aqueous sodium bicarbonate and extracted with EtOAc. The organic layer was washed with water, dried over sodium sulfate, filtered and concentrated under reduced pressure. The crude product was purified by preparative HPLC (X TERRA C18, 19×250 mm×10 μm, flow rate: 20 mL/min, 0.1% TFA in water with MeCN 20% to 70% over 9 min, held at 70% for 5 min then ramped to 100% over 0.1 min and held for 2.9 min) to provide the title compound (40 mg). 1H NMR (400 MHZ; DMSO-d₆) δ: 10.01 (br s, 1H), 8.94 (d, 1H), 8.61 (d, 1H), 8.17 (s, 2H), 7.61 (dd, 1H), 7.34 (dd, 1H), 6.85 (t, 1H) 4.43 (t, 2H), 3.95 (t, 2H). LCMS (Method 1): 2.01 min, 467.2 [M−H]⁻.

### Example 5: N-(6-(2H-1,2,3-Triazol-2-yl)-5-(trifluoromethyl)pyridin-3-yl)-8-bromo-2-methyl-2,3-dihydro-4H-benzo[b][1,4]oxazine-4-carboxamide

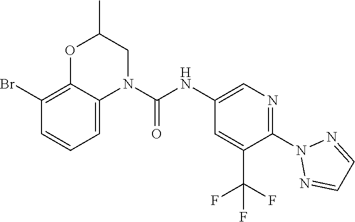 [SMILES: CC1CN(C(=O)Nc2cnc(-n3nccn3)c(C(F)(F)F)c2)c2cccc(Br)c2O1]

To a stirred solution of 8-bromo-2-methyl-3,4-dihydro-2H-benzo[b][1,4]oxazine (0.20 g, 0.87 mmol, CAS 1267095-78-2) in THF (5 mL) at 0° C. was added 6-(2H-1,2,3-triazol-2-yl)-5-(trifluoromethyl)pyridin-3-amine (0.20 g, 0.87 mmol, CAS 1832582-59-8) and triphosgene (0.13 g, 0.44 mmol) and stirred at 0° C. for 15 min. Then triethylamine (0.25 mL, 1.75 mmol) was added dropwise and the mixture was stirred at RT for 16 h. The mixture was diluted with DCM and washed with saturated aqueous sodium bicarbonate, water, dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN 25% to 50% over 7 min, held at 50% for 6.1 min then ramped to 100% over 0.1 min and held for 3.8 min) to provide the title compound (37 mg). 1H NMR (400 MHZ; DMSO-d₆) δ: 10.02 (s, 1H), 8.94 (d, 1H), 8.60 (d, 1H), 8.17 (s, 2H), 7.60 (dd, 1H), 7.34 (dd, 1H), 6.85 (t, 1H), 4.48-4.52 (m, 1H), 4.18 (dd, 1H), 3.45 (dd, 1H), 1.38 (d, 3H). LCMS (Method 7): 2.47 min, 483.1 [M+H]⁺.

### Example 6: N-(6-(2H-1,2,3-Triazol-2-yl)-5-(trifluoromethyl)pyridin-3-yl)-8-bromo-2-cyano-2,3-dihydro-4H-benzo[b][1,4]oxazine-4-carboxamide

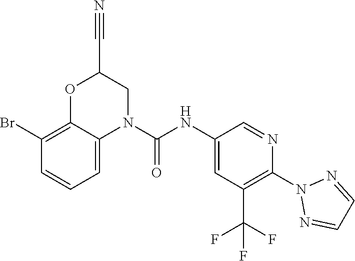 [SMILES: N#CC1CN(C(=O)Nc2cnc(-n3nccn3)c(C(F)(F)F)c2)c2cccc(Br)c2O1]

To a stirred solution of Intermediate 1 (0.30 g, 1.25 mmol) in DCM (2 mL) at 0° C. was added 6-(2H-1,2,3-triazol-2-yl)-5-(trifluoromethyl)pyridin-3-amine (0.29 g, 1.25 mmol, CAS 1832582-59-8) and triphosgene (0.19 g, 0.63 mmol) and stirred at 0° C. for 15 min. Then triethylamine (0.35 g, 3.50 mmol) was added dropwise and the mixture was stirred at RT for 4 h. The mixture was diluted with DCM and washed with saturated aqueous sodium bicarbonate, water, dried over sodium sulfate, filtered and concentrated under reduced pressure. The crude product was purified by preparative HPLC (X-Bridge C18, 19×250 mm×5 μm, flow rate: 20 mL/min, 10 mM NH₄HCO₃ in water with MeCN 30% to 50% over 4 min, held at 50% for 16 min then ramped to 100% over 0.1 min and held for 2.9 min) to provide the title compound (50 mg). 1H NMR (400 MHZ; DMSO-d₆) δ: 10.29 (s, 1H), 8.95 (br s, 1H), 8.60 (d, 1H), 8.17 (s, 2H), 7.63 (br d, 1H), 7.45 (dd, 1H), 7.01 (t, 1H), 5.94 (t, 1H), 4.66 (dd, 1H), 3.93 (dd, 1H). LCMS (Method 1): 1.94 min, 492.1 [M−H]⁻.

### Example 7: N-(5-Chloro-6-(2H-1,2,3-triazol-2-yl)pyridin-3-yl)-5-cyano-3,4-dihydroquinoline-1 (2H)-carboxamide

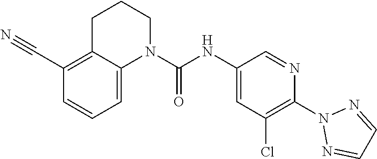 [SMILES: N#Cc1cccc2c1CCCN2C(=O)Nc1cnc(-n2nccn2)c(Cl)c1]

To a stirred solution of 5-chloro-6-(2H-1,2,3-triazol-2-yl)pyridin-3-amine (0.20 g, 1.02 mmol, CAS 1832583-43-3) in DCM (2 mL) at 0° C. was added triethylamine (0.28 mL, 2.04 mmol) and triphosgene (0.37 g, 1.02 mmol) and stirred at 0° C. for 30 min. 1,2,3,4-Tetrahydroquinoline-5-carbonitrile (0.17 g, 1.02 mmol, CAS 939758-72-2) was added then the mixture was stirred at RT for 16 h. The mixture was diluted with DCM and washed with saturated aqueous sodium bicarbonate, water, dried over sodium sulfate, filtered and concentrated under reduced pressure. The crude product was purified by preparative HPLC (X TERRA C18, 19×250 mm×10 μm, flow rate: 20 mL/min, 0.1% TFA in water with MeCN 20% to 70% over 9 min, held at 70% for 5 min then ramped to 100% over 0.1 min and held for 1.9 min) to provide the title compound (39 mg). 1H NMR (400 MHZ; DMSO-d₆) δ: 9.76 (s, 1H), 8.64 (d, 1H), 8.37 (d, 1H), 8.15 (s, 2H), 7.81 (dd, 1H), 7.55 (dd, 1H), 7.35 (t, 1H), 3.81 (t, 2H), 2.95 (t, 2H), 1.98-2.06 (m, 2H). LCMS (Method 1): 1.75 min, 378.3 [M−H]⁻.

### Example 8: N-(6-(2H-1,2,3-Triazol-2-yl)-5-(trifluoromethyl)pyridin-3-yl)-5-(pyridin-3-yl)-3,4-dihydroquinoline-1 (2H)-carboxamide

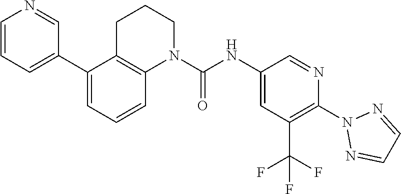 [SMILES: O=C(Nc1cnc(-n2nccn2)c(C(F)(F)F)c1)N1CCCc2c(-c3cccnc3)cccc21]

To a stirred solution of Intermediate 3 (0.10 g, 0.47 mmol) in DCM (10 mL) at 0° C. was added 6-(2H-1,2,3-triazol-2-yl)-5-(trifluoromethyl)pyridin-3-amine (0.11 g, 0.47 mmol, CAS 1832582-59-8), triphosgene (75 mg, 0.23 mmol) and triethylamine (0.1 mL, 0.72 mmol) then the mixture was stirred at RT for 2 h. The mixture was diluted with water and extracted with EtOAc. The organic layer was washed with saturated aqueous sodium bicarbonate, brine, dried over sodium sulfate, filtered and concentrated under reduced pressure. The crude product was purified by preparative HPLC (X-Bridge Phenyl, 19×250 mm×5 μm, flow rate: 18 mL/min, 0.1% TFA in water with MeCN 10% to 40% over 7 min, held at 40% for 4.5 min then ramped to 100% over 0.1 min and held for 2.4 min) to provide the title compound (22 mg). 1H NMR (400 MHZ; DMSO-d₆) δ: 9.76 (br s, 1H), 8.99 (d, 1H), 8.66-8.69 (m, 3H), 8.17 (s, 2H), 7.96 (d, 1H), 7.63 (dd, 1H), 7.57 (d, 1H), 7.33 (t, 1H), 7.09 (d, 1H), 3.79 (t, 2H), 2.62 (t, 2H), 1.87-1.91 (m, 2H). LCMS (Method 8): 2.23 min, 466.4 [M+H]⁺.

### Example 9: N-(6-(2H-1,2,3-Triazol-2-yl)-5-(trifluoromethyl)pyridin-3-yl)-5-(oxazol-2-yl)-3,4-dihydroquinoline-1 (2H)-carboxamide

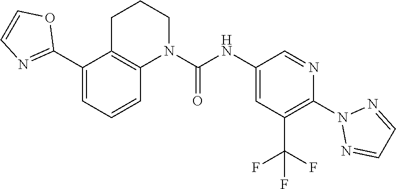 [SMILES: O=C(Nc1cnc(-n2nccn2)c(C(F)(F)F)c1)N1CCCc2c(-c3ncco3)cccc21]

To a stirred solution of Intermediate 5 (0.13 g, 0.65 mmol) in DCM (5 mL) at 0° C. was added 6-(2H-1,2,3-triazol-2-yl)-5-(trifluoromethyl)pyridin-3-amine (0.15 g, 0.65 mmol, CAS 1832582-59-8) and triphosgene (135 mg, 0.45 mmol) and the mixture stirred at 0° C. for 15 min. Triethylamine (0.27 mL, 1.95 mmol) was added dropwise then the mixture was stirred at RT for 16 h. The mixture was diluted with DCM then washed with saturated aqueous sodium bicarbonate, water, dried over sodium sulfate, filtered and concentrated under reduced pressure. The crude product was purified by preparative HPLC (X-SELECT C18, 19×250 mm×5 μm, flow rate: 18 mL/min, 10 mM NH₄HCO₃ in water with MeCN, 35% to 65% over 4 min, held at 65% for 12 min then ramped to 100% over 1 min and held for 5 min) to provide the title compound (46 mg). 1H NMR (400 MHZ; DMSO-d₆) δ: 9.76 (br s, 1H), 8.93 (d, 1H), 8.62 (d, 1H), 8.27 (d, 1H), 8.16 (s, 2H), 7.68 (dd, 1H), 7.61 (d, 1H), 7.44 (d, 1H), 7.35 (t, 1H), 3.80 (t, 2H), 3.32 (t, 2H), 1.94-1.99 (m, 2H). LCMS (Method 1): 2.63 min, 454.0 [M−H]⁻.

### Example 10: N-(6-(2H-1,2,3-Triazol-2-yl)-5-(trifluoromethyl)pyridin-3-yl)-5-(thiazol-2-yl)-3,4-dihydroquinoline-1 (2H)-carboxamide

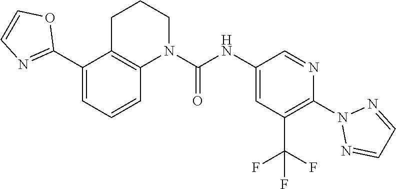 [SMILES: O=C(Nc1cnc(-n2nccn2)c(C(F)(F)F)c1)N1CCCc2c(-c3ncco3)cccc21]

To a stirred solution of Intermediate 7 (0.15 g, 0.69 mmol) in DCM (5 mL) at 0° C. was added 6-(2H-1,2,3-triazol-2-yl)-5-(trifluoromethyl)pyridin-3-amine (0.15 g, 0.65 mmol, CAS 1832582-59-8) and triphosgene (0.14 g, 0.48 mmol) and the mixture stirred at 0° C. for 15 min. Triethylamine (0.29 mL, 2.08 mmol) was added dropwise then the mixture was stirred at RT for 16 h. The mixture was diluted with DCM then washed with saturated aqueous sodium bicarbonate, water, dried over sodium sulfate, filtered and concentrated under reduced pressure. The crude product was purified by preparative HPLC (X-SELECT C18, 19×250 mm×5 μm, flow rate: 18 mL/min, 10 mM NH₄HCO₃ in water with MeCN, 30% to 55% over 4 min, held at 55% for 8.52 min then ramped to 100% over 0.1 min and held for 3.4 min) to provide the title compound (45 mg). 1H NMR (400 MHZ; DMSO-d₆) δ: 9.77 (br s, 1H), 8.95 (d, 1H), 8.63 (d, 1H), 8.16 (s, 2H), 8.01 (d, 1H), 7.89 (d, 1H), 7.59 (dd, 1H), 7.44 (dd, 1H), 7.33 (t, 1H), 3.79 (t, 2H), 3.03 (t, 2H), 1.89-1.96 (m, 2H). LCMS (Method 8): 2.38 min, 472.3 [M+H]⁺.

### Example 11: cis-N-(6-(2H-1,2,3-Triazol-2-yl)-5-(trifluoromethyl)pyridin-3-yl)-5-bromo-3,4-dimethyl-3,4-dihydroquinoline-1 (2H)-carboxamide

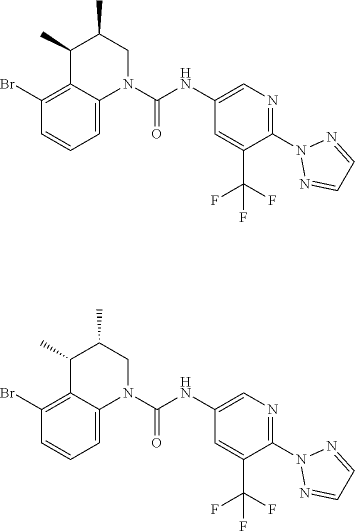 [SMILES: C[C@@H]1c2c(Br)cccc2N(C(=O)Nc2cnc(-n3nccn3)c(C(F)(F)F)c2)C[C@@H]1C ; C[C@@H]1CN(C(=O)Nc2cnc(-n3nccn3)c(C(F)(F)F)c2)c2cccc(Br)c2[C@@H]1C]

To a stirred solution of Intermediate 10 (0.27 g, 1.10 mmol) in DCM (10 mL) at 0° C. was added 6-(2H-1,2,3-triazol-2-yl)-5-(trifluoromethyl)pyridin-3-amine (0.25 g, 1.10 mmol, CAS 1832582-59-8) and triphosgene (0.24 g, 0.82 mmol) and the mixture stirred at 0° C. for 15 min. Triethylamine (0.22 mL, 1.65 mmol) was added dropwise then the mixture was stirred at RT for 2 h. The mixture was diluted with DCM then washed with saturated aqueous sodium bicarbonate, brine, dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 40% to 70% over 5 min, held at 70% for 8 min then ramped to 100% over 0.1 min and held for 2.9 min) to provide the title compound (60 mg). 1H NMR (400 MHZ; DMSO-d₆) δ: 9.67 (br s, 1H), 8.99 (d, 1H), 8.63 (d, 1H), 8.16 (s, 2H), 7.55 (d, 1H), 7.34 (dd, 1H), 7.12 (t, 1H), 3.90 (dd, 1H), 3.37 (d, 1H), 3.10-3.18 (m, 1H), 2.01-2.12 (m, 1H), 1.12 (d, 3H), 0.96 (d, 3H). LCMS (Method 1): 2.36 min, 493.2 [M−H]⁻.

### Example 12: N-(6-(2H-1,2,3-Triazol-2-yl)-5-(trifluoromethyl)pyridin-3-yl)-5-phenyl-3,4-dihydroquinoline-1 (2H)-carboxamide

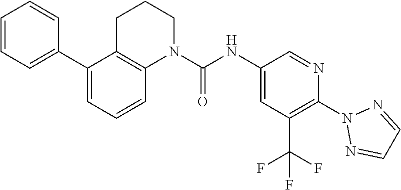 [SMILES: O=C(Nc1cnc(-n2nccn2)c(C(F)(F)F)c1)N1CCCc2c(-c3ccccc3)cccc21]

To a stirred solution of Example 2 (0.27 g, 1.10 mmol) in 1,4-dioxane:water (4:1 10 mL) was added Cs₂CO₃ (0.39 g, 1.2 mmol) and phenyl boronic acid (70 mg, 0.64 mmol, CAS 98-80-6) and the mixture was degassed with nitrogen for 10 min. PdCl₂ (PPh₃)₂ (21 mg, 0.03 mmol) and X-Phos (17 mg, 0.03 mmol) were added and the mixture was degassed with nitrogen for 10 min. The mixture stirred at 90° C. for 16 h. The mixture was diluted with water and extracted with EtOAc. The organic layer was washed with saturated aqueous sodium bicarbonate, brine, dried over sodium sulfate, filtered and concentrated under reduced pressure. The crude product was purified by preparative HPLC (X-SELECT

C18, 19×250 mm×5 μm, flow rate: 18 mL/min, 10 mM NH₄HCO₃ in water with MeCN, 40% to 60% over 4 min, held at 60% for 16.5 min then ramped to 100% over 0.1 min and held for 3.4 min) to provide the title compound (21 mg). 1H NMR (400 MHZ; DMSO-d₆) δ: 9.71 (br s, 1H), 8.98 (d, 1H), 8.65 (d, 1H), 8.16 (s, 2H), 7.48 (t, 3H), 7.41 (d, 1H), 7.37 (d, 2H), 7.27 (t, 1H), 7.04 (dd, 1H), 3.76 (t, 2H), 2.61 (t, 2H), 1.83-1.90 (m, 2H). LCMS (Method 1): 2.29 min, 463.3 [M−H]⁻.

### Example 13:8-Bromo-N-(5-chloro-6-(2H-1,2,3-triazol-2-yl)pyridin-3-yl)-2,2-dimethyl-2,3-dihydro-4H-benzo[b][1,4]oxazine-4-carboxamide

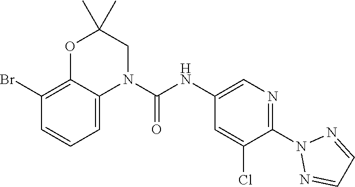 [SMILES: CC1(C)CN(C(=O)Nc2cnc(-n3nccn3)c(Cl)c2)c2cccc(Br)c2O1]

To a stirred solution of 5-chloro-6-(2H-1,2,3-triazol-2-yl) nicotinic acid (0.25 g, 1.03 mmol, CAS 2244109-97-3) in toluene (10 mL) was added triethylamine (0.28 mL, 2.04 mmol) and DPPA (0.3 mL, 1.24 mmol) and the mixture was stirred at 100° C. for 30 min. 8-Bromo-2,2-dimethyl-3,4-dihydro-2H-benzo[b][1,4]oxazine (0.20 g, 0.83 mmol, CAS 853683-76-8) was added then the mixture was stirred at 100° C. for 16 h. The mixture was cooled and quenched with water then extracted with EtOAc. The organic layer was dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 10% to 67% over 1.14 min, then to 75% over 8.44 min, then to 100% over 1.5 min and held for 2 min) to provide the title compound (45 mg). 1H NMR (400 MHZ; DMSO-d₆) δ: 9.95 (s, 1H), 8.63 (d, 1H), 8.36 (d, 1H), 8.15 (s, 2H), 7.52 (dd, 1H), 7.35 (dd, 1H), 6.85 (t, 1H), 3.73 (s, 2H), 1.36 (s, 6H). LCMS (Method 9): 3.71 min, 463.5 [M+H]⁺.

### Example 14: N-(6-(2H-1,2,3-triazol-2-yl)-5-(trifluoromethyl)pyridin-3-yl)-5-(1,3,4-thiadiazol-2-yl)-3,4-dihydroquinoline-1 (2H)-carboxamide

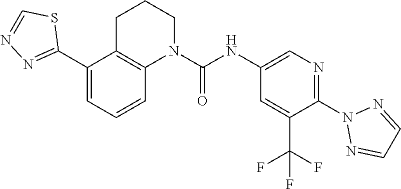 [SMILES: O=C(Nc1cnc(-n2nccn2)c(C(F)(F)F)c1)N1CCCc2c(-c3nncs3)cccc21]

To a stirred solution of Intermediate 13 (0.13 g, 0.58 mmol) in DCM (10 mL) at 0° C. was added DIPEA (0.22 g, 1.73 mmol) and 6-(2H-1,2,3-triazol-2-yl)-5-(trifluoromethyl)pyridin-3-amine (0.13 g, 0.58 mmol, CAS 1832582-59-8) and mixture stirred at 0° C. for 15 min. Phosgene (0.28 g, 0.57 mmol) was added at 0° C. and then the mixture was stirred at RT for 5 h. The mixture was quenched with water and extracted with DCM. The organic layer was dried over sodium sulfate, filtered and concentrated under reduced pressure. The crude product was purified by preparative HPLC (X-SELECT C18, 19×250 mm×5 μm, flow rate: 18 mL/min, 10 mM NH₄HCO₃ in water with MeCN, 45% to 85% over 9 min, then to 90% over 5 min and ramped to 100% over 0.1 min and held for 2.9 min) to provide the title compound (16 mg). 1H NMR (400 MHZ; DMSO-d₆) δ: 9.81 (br s, 1H), 9.75 (s, 1H), 8.95 (d, 1H), 8.63 (d, 1H), 8.16 (s, 2H), 7.67 (dd, 1H), 7.43 (dd, 1H), 7.36 (t, 1H), 3.81 (t, 2H), 2.94 (t, 2H), 1.91-1.98 (m, 2H). LCMS (Method 10): 2.12 min, 473.3 [M+H]⁺.

### Example 15:5-Bromo-N-(5-chloro-6-(2H-1,2,3-triazol-2-yl)pyridin-3-yl)-3,3-dimethyl-3,4-dihydroquinoline-1 (2H)-carboxamide

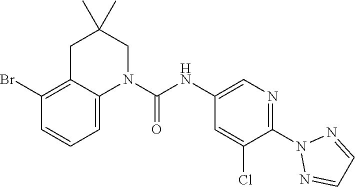 [SMILES: CC1(C)Cc2c(Br)cccc2N(C(=O)Nc2cnc(-n3nccn3)c(Cl)c2)C1]

To a stirred solution of Intermediate 16 (0.30 g, 1.25 mmol) and 5-chloro-6-(2H-1,2,3-triazol-2-yl)pyridin-3-amine (0.15 g, 0.75 mmol, CAS 1832583-43-3) in DCM (10 mL) at 0° C. was added triethylamine (0.28 mL, 2.04 mmol). Triphosgene (0.28 g, 0.94 mmol) was added then the mixture was stirred at RT for 3 h. The mixture diluted with DCM and washed with water, dried over sodium sulfate, filtered and concentrated under reduced pressure. The crude product was purified by preparative HPLC (X-Bridge C18, 19×150 mm×5 μm, flow rate: 18 mL/min, 10 mM NH₄HCO₃ in water with MeCN, 40% to 75% over 4 min, held at 75% for 6 min) to provide the title compound (56 mg). 1H NMR (400 MHZ; CDCl₃) δ: 8.51 (d, 1H), 8.26 (d, 1H), 7.91 (s, 2H), 7.47 (dd, 1H), 7.30 (d, 2H), 7.17 (t, 1H), 3.62 (s, 2H), 2.66 (s, 2H), 1.09 (s, 6H). LCMS (Method 14): 2.24 min, 459.1 [M−H]⁻.

### Example 16:5-Bromo-N-(5-chloro-6-(2H-1,2,3-triazol-2-yl)pyridin-3-yl)-4-methyl-3,4-dihydroquinoxaline-1 (2H)-carboxamide

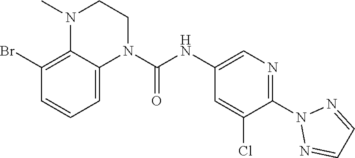 [SMILES: CN1CCN(C(=O)Nc2cnc(-n3nccn3)c(Cl)c2)c2cccc(Br)c21]

To a stirred solution of Intermediate 17 (0.30 g, 1.32 mmol) and 5-chloro-6-(2H-1,2,3-triazol-2-yl)pyridin-3-amine (0.18 g, 0.92 mmol, CAS 1832583-43-3) in DCM (10 mL) at 0° C. was added triethylamine (0.53 g, 5.28 mmol). Triphosgene (0.27 g, 0.92 mmol) was added then the mixture was stirred at RT for 3 h. The mixture diluted with DCM and washed with water, brine, dried over sodium sulfate, filtered and concentrated under reduced pressure. The crude product was purified by flash column chromatography (silica gel, 100-22 mesh, eluting 15-20% EtOAc in hexane) followed by preparative HPLC (X-Bridge C18, 19×250 mm×5 μm, flow rate: 18 mL/min, 10 mM NH₄HCO₃ in water with MeCN, 25% to 55% over 4 min, held at 55% for 6.8 min, then to 100% over 4 min) to provide the title compound (30 mg). 1H NMR (400 MHz; DMSO-d₆) δ: 9.78 (s, 1H), 8.65 (d, 1H), 8.39 (d, 1H), 8.15 (s, 2H), 7.57 (dd, 1H), 7.30 (dd, 1H), 6.87 (t, 1H), 3.84 (dd, 2H), 3.20 (dd, 2H), 2.87 (s, 3H). LCMS (Method 1): 1.97 min, 446.2 [M−H]⁻.

### Example 17:5-Bromo-N-(5-chloro-6-(2H-1,2,3-triazol-2-yl)pyridin-3-yl)-3,4-dihydro-1,7-naphthyridine-1 (2H)-carboxamide

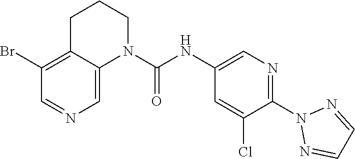 [SMILES: O=C(Nc1cnc(-n2nccn2)c(Cl)c1)N1CCCc2c(Br)cncc21]

To a stirred solution of 5-bromo-1,2,3,4-tetrahydro-1,7-naphthyridine (95 mg, 0.44 mmol, CAS 351457-97-1) and 5-chloro-6-(2H-1,2,3-triazol-2-yl)pyridin-3-amine (51 mg, 0.26 mmol, CAS 1832583-43-3) in DCM (2 mL) at 0° C. was added triethylamine (0.18 mL, 1.32 mmol). Triphosgene (91 mg, 0.31 mmol) was added then the mixture was stirred at RT for 16 h. The mixture was quenched with water and extracted with 10% MeOH in DCM. The combined organic layer was dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 20% to 60% over 7 min, held at 60% for 4.8 min, ramped to 100% over 0.2 min and held at 100% for 5 min) to provide the title compound (9 mg). 1H NMR (400 MHZ; CDCl₃) δ: 8.59 (s, 1H), 8.54 (s, 1H), 8.47 (d, 1H), 8.31 (d, 1H), 7.92 (s, 2H), 7.15 (s, 1H), 3.89 (t, 2H), 2.90 (t, 2H), 2.06-2.13 (m, 2H). LCMS (Method 1): 1.66 min, 434.2 [M+H]⁺.

### Example 18:8-Bromo-N-(5-chloro-6-(2H-1,2,3-triazol-2-yl)pyridin-3-yl)-2-cyano-2-methyl-2,3-dihydro-4H-benzo[b][1,4]oxazine-4-carboxamide

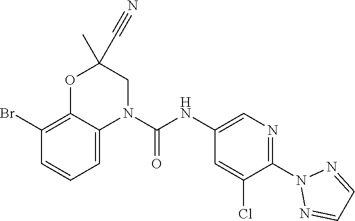 [SMILES: CC1(C#N)CN(C(=O)Nc2cnc(-n3nccn3)c(Cl)c2)c2cccc(Br)c2O1]

To a stirred solution of Intermediate 20 (0.30 g, 1.18 mmol) and 5-chloro-6-(2H-1,2,3-triazol-2-yl)pyridin-3-amine (0.16 g, 0.83 mmol, CAS 1832583-43-3) in DCM (7 mL) at 0° C. was added triethylamine (0.36 g, 3.54 mmol). Triphosgene (0.25 g, 0.83 mmol) was added then the mixture was stirred at RT for 2 h. Solvent was removed under reduced pressure and the residue partitioned between EtOAc and water. The organic layer was dried over sodium sulfate, filtered and concentrated under reduced pressure. The crude product was purified by preparative HPLC (X-Bridge Phenyl, 19×250 mm×5 μm, flow rate: 18 mL/min, 10 mM NH₄HCO₃ in water with MeCN, 35% to 50% over 4 min, held at 50% for 10 min) to provide the title compound (50 mg). 1H NMR (400 MHZ; DMSO-d₆) δ: 10.19 (s, 1H), 8.65 (d, 1H), 8.35 (d, 1H), 8.16 (s, 2H), 7.60 (d, 1H), 7.46 (dd, 1H), 7.01 (t, 1H), 4.89 (d, 1H), 3.60 (d, 1H), 1.88 (s, 3H). LCMS (Method 11): 2.38 min, 474.1 [M+H]⁺.

### Example 19:8-Bromo-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide

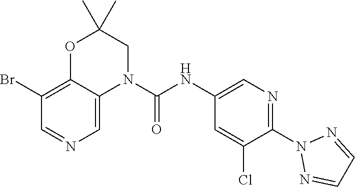 [SMILES: CC1(C)CN(C(=O)Nc2cnc(-n3nccn3)c(Cl)c2)c2cncc(Br)c2O1]

To a stirred solution of Intermediate 23 (1.50 g, 6.17 mmol) and 5-chloro-6-(2H-1,2,3-triazol-2-yl)pyridin-3-amine (1.20 g, 6.17 mmol, CAS 1832583-43-3) in DCM (15 mL) at 0° C. was added triethylamine (3.44 mL, 24.7 mmol) then the mixture was allowed to RT for 30 min. The mixture was cooled again to 0° C. and triphosgene (1.83 g, 6.17 mmol) was added as a solution in DCM (10 mL) dropwise then the mixture was stirred at RT for 2 h. Solvent was removed under reduced pressure. The crude product was purified by preparative HPLC (Symmetry C18, 19×250 mm×5 μm, flow rate: 17 mL/min, water with MeCN, Isocratic 50:50) to provide the title compound (0.51 g). 1H NMR (400 MHZ; DMSO-d₆) δ: 10.08 (br s, 1H), 8.67 (s, 1H), 8.61 (s, 1H), 8.37 (d, 1H), 8.35 (s, 1H), 8.15 (s, 2H), 3.82 (s, 2H), 1.39 (s, 6H). LCMS (Method 4): 1.78 min, 464.4 [M+H]⁺.

### Example 20:8-Bromo-N-(5-chloro-6-(2H-1,2,3-triazol-2-yl)pyridin-3-yl)-2-ethynyl-2,3-dihydro-4H-benzo[b][1,4]oxazine-4-carboxamide

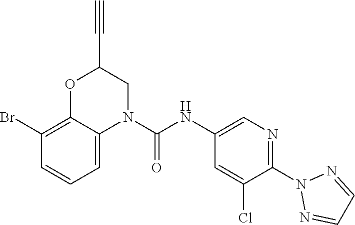 [SMILES: C#CC1CN(C(=O)Nc2cnc(-n3nccn3)c(Cl)c2)c2cccc(Br)c2O1]

To a stirred solution of Intermediate 28 (0.19 g, 0.67 mmol) and 5-chloro-6-(2H-1,2,3-triazol-2-yl)pyridin-3-amine (92 mg, 0.47 mmol, CAS 1832583-43-3) in DCM (7 mL) at 0° C. was added triethylamine (0.28 mL, 2.01 mmol). Triphosgene (0.14 g, 0.47 mmol) was added then the mixture was stirred at RT for 1 h. The mixture diluted with DCM and washed with brine, dried over sodium sulfate, filtered and concentrated under reduced pressure. The crude product was purified by preparative HPLC (X-Bridge C18, 30×150 mm×5 μm, flow rate: 18 mL/min, 10 mM NH₄HCO₃ in water with MeCN, 30% to 50% over 6 min, held at 50% for 5 min, ramped to 100% over 0.1 min, held at 100% 5.9 min) to provide the title compound (10 mg). 1H NMR (400 MHZ; DMSO-d₆) δ: 9.97 (br s, 1H), 8.65 (s, 1H), 8.36 (d, 1H), 8.15 (s, 2H), 7.55 (d, 1H), 7.38 (d, 1H), 6.91 (t, 1H), 5.47 (s, 1H), 4.24 (dd, 1H), 3.83 (d, 1H), 3.76 (d, 1H). LCMS (Method 12): 2.05 min, 459.3 [M+H]⁺.

### Example 21:8-Bromo-N-(5-chloro-6-(2H-1,2,3-triazol-2-yl)pyridin-3-yl)-2,3-dihydro-4H-pyrido[4,3-b][1,4]oxazine-4-carboxamide

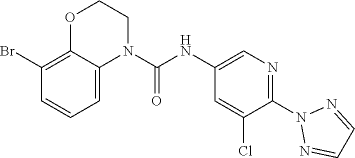 [SMILES: O=C(Nc1cnc(-n2nccn2)c(Cl)c1)N1CCOc2c(Br)cccc21]

To a stirred solution of 8-bromo-3,4-dihydro-2H-pyrido[4,3-b][1,4]oxazine (0.13 g, 0.58 mmol, CAS 1379320-13-4) and 5-chloro-6-(2H-1,2,3-triazol-2-yl)pyridin-3-amine (79 mg, 0.40 mmol, CAS 1832583-43-3) in DCM (5 mL) at 0° C. was added triethylamine (0.24 g, 1.74 mmol). Triphosgene (0.12 g, 0.40 mmol) was added then the mixture was stirred at RT for 16 h. The mixture was quenched with water and extracted with 10% MeOH in DCM. The combined organic layer was dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 20% to 60% over 7 min, held at 60% for 4.8 min, ramped to 100% over 0.2 min, held at 100% 5 min) to provide the title compound (30 mg). 1H NMR (400 MHZ; DMSO-d₆) δ: 10.01 (br s, 1H), 8.84 (s, 1H), 8.50 (s, 1H), 8.39 (d, 1H), 8.27 (s, 1H), 8.13 (s, 2H), 4.48 (t, 2H), 4.03 (t, 2H). LCMS (Method 13): 1.94 min, 436.1 [M+H]⁺.

### Example 22: N-(5-Chloro-6-(2H-1,2,3-triazol-2-yl)pyridin-3-yl)-5-cyclopropyl-3,4-dihydro-1,7-naphthyridine-1 (2H)-carboxamide

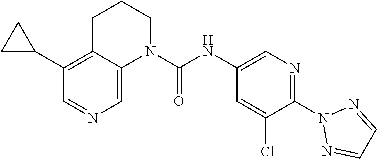 [SMILES: O=C(Nc1cnc(-n2nccn2)c(Cl)c1)N1CCCc2c(C3CC3)cncc21]

To a stirred solution of Intermediate 30 (0.17 g, 0.97 mmol) and 5-chloro-6-(2H-1,2,3-triazol-2-yl)pyridin-3-amine (0.19 g, 0.97 mmol, CAS 1832583-43-3) in DCM (10 mL) at 0° C. was added triethylamine (0.30 g, 2.92 mmol). Triphosgene (0.22 g, 0.73 mmol) was added then the mixture was stirred at RT for 3 h. The mixture was quenched with saturated aqueous sodium bicarbonate and extracted with DCM. The combined organic layer was washed with brine, dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 10% to 70% over 14 min, ramped to 100% over 0.1 min, held at 100% 1.9 min) to provide the title compound (10 mg). 1H NMR (400 MHZ; DMSO-d₆) δ: 9.75 (br s, 1H), 8.62 (d, 1H), 8.51 (s, 1H), 8.38 (d, 1H), 8.14 (s, 2H), 7.94 (s, 1H), 3.80 (t, 2H), 2.91 (t, 2H), 1.97-2.01 (m, 2H), 1.84-1.88 (m, 1H), 0.94-0.98 (m, 2H), 0.70-0.73 (m, 2H). LCMS (Method 1): 1.30 min, 396.2 [M+H]⁺.

### Example 23: N-(6-(2H-1,2,3-Triazol-2-yl)-5-(trifluoromethyl)pyridin-3-yl)-8-bromo-2,2-dimethyl-2,3-dihydro-4H-benzo[b][1,4]oxazine-4-carboxamide

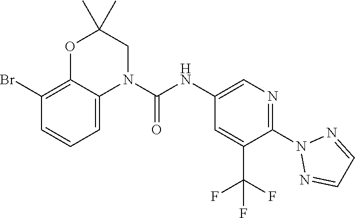 [SMILES: CC1(C)CN(C(=O)Nc2cnc(-n3nccn3)c(C(F)(F)F)c2)c2cccc(Br)c2O1]

To a stirred solution of 6-(2H-1,2,3-triazol-2-yl)-5-(trifluoromethyl)pyridin-3-amine (0.19 g, 0.82 mmol, CAS 1832582-59-8) and 8-bromo-2,2-dimethyl-3,4-dihydro-2H-benzo[b][1,4]oxazine (0.25 g, 1.03 mmol, CAS 853683-76-8) in DCM (15 mL) at 0° C. was added triethylamine (0.43 mL, 3.09 mmol) and triphosgene (0.21 g, 0.72 mmol). The mixture was stirred at RT for 1 h. The mixture was diluted with water then extracted with DCM. The organic layer was dried over sodium sulfate, filtered and concentrated under reduced pressure. The crude product was purified by preparative HPLC (X-Bridge C18, 19×250 mm×5 μm, flow rate: 18 mL/min, 10 mM NH₄HCO₃ in water with MeCN, 10% to 67% over 1.14 min, then to 75% over 8.44 min, then to 100% over 1.5 min and held for 2 min) to provide the title compound (50 mg). 1H NMR (400 MHZ; DMSO-d₆) δ: 10.08 (s, 1H), 8.94 (d, 1H), 8.59 (d, 1H), 8.16 (s, 2H), 7.55 (dd, 1H), 7.36 (dd, 1H), 6.86 (t, 1H), 3.75 (s, 2H), 1.36 (s, 6H). LCMS (Method 1): 2.21 min, 495.2 [M−H]⁻.

### Example 24: N-(5-Chloro-6-(2H-1,2,3-triazol-2-yl)pyridin-3-yl)-8-cyclopropyl-2,2-dimethyl-2,3-dihydro-4H-pyrido[4,3-b][1,4]oxazine-4-carboxamide

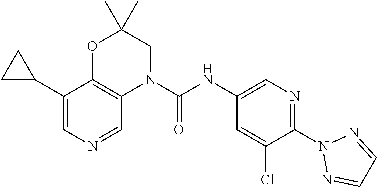 [SMILES: CC1(C)CN(C(=O)Nc2cnc(-n3nccn3)c(Cl)c2)c2cncc(C3CC3)c2O1]

To a stirred solution of Intermediate 32 (0.10 g, 0.49 mmol) and 5-chloro-6-(2H-1,2,3-triazol-2-yl)pyridin-3-amine (67 mg, 0.34 mmol, CAS 1832583-43-3) in DCM (5 mL) at 0° C. was added pyridine (0.12 mL, 1.47 mmol). Triphosgene (0.22 g, 0.73 mmol) was added as a solution in DCM (1 mL) and the reaction was stirred at 0° C. for 15 min then at RT for 16 h. The mixture was concentrated under reduced pressure. The crude product was purified by preparative HPLC (X-Bridge C18, 19×250 mm×5 μm, flow rate: 17 mL/min, 10 mM NH₄HCO₃ in water with MeCN, 30% to 50% over 8 min, ramped to 98% over 0.1 min, held at 98% 1.9 min) to provide the title compound (39 mg). 1H NMR (400 MHz; DMSO-d₆) δ: 10.03 (br s, 1H), 8.60 (br s, 1H), 8.48 (br s, 1H), 8.37 (d, 1H), 8.12 (s, 2H), 7.72 (s, 1H), 3.79 (s, 2H), 1.93-2.00 (m, 1H), 1.35 (s, 6H), 0.91-0.94 (m, 2H), 0.74-0.77 (m, 2H). LCMS (Method 14): 1.52 min, 426.5 [M+H]⁺.

### Example 25: (S)-1-Bromo-N-(5-chloro-6-(2H-1,2,3-triazol-2-yl)pyridin-3-yl)-6a,7,8,9-tetrahydropyrido[3,4-e] pyrrolo[1,2-a]pyrazine-5 (6H)-carboxamide

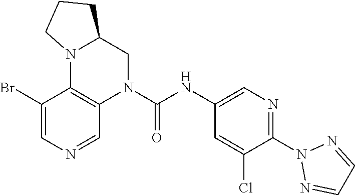 [SMILES: O=C(Nc1cnc(-n2nccn2)c(Cl)c1)N1C[C@@H]2CCCN2c2c(Br)cncc21]

To a stirred solution of Intermediate 35 (0.30 g, 1.18 mmol) and 5-chloro-6-(2H-1,2,3-triazol-2-yl)pyridin-3-amine (0.18 g, 0.92 mmol, CAS 1832583-43-3) in DCM (5 mL) at 0° C. was added pyridine (0.29 mL, 3.54 mmol). Triphosgene (0.35 g, 1.18 mmol) was added as a solution in DCM (1 mL) and stirred at 0° C. for 15 min then at RT for 3 h. The mixture was concentrated under reduced pressure. The crude product was purified by preparative HPLC (X-Select C18, 25×150 mm×5 μm, flow rate: 18 mL/min, 10 mM NH₄HCO₃ in water with MeCN, 25% to 45% over 6 min, held at 45% for 8 min, ramped to 98% over 0.1 min, held at 98% 1.9 min) to provide the title compound (50 mg). 1H NMR (400 MHz; DMSO-d₆) δ: 9.75 (br s, 1H), 8.58 (s, 1H), 8.35 (d, 1H), 8.33 (s, 1H), 8.18 (s, 1H), 8.14 (s, 2H), 4.43 (d, 1H), 4.17-4.23 (m, 1H), 3.40-3.54 (m, 2H), 2.70 (dd, 1H), 2.11-2.20 (m, 1H), 1.83-1.90 (m, 2H), 1.69-1.69 (m, 1H). LCMS (Method 14): 1.41 min, 475.5 [M+H]⁺.

### Example 26: (R)-8-Bromo-N-(5-chloro-6-(2H-1,2,3-triazol-2-yl)pyridin-3-yl)-1.2-dimethyl-2,3-dihydropyrido[3,4-b]pyrazine-4 (1H)-carboxamide

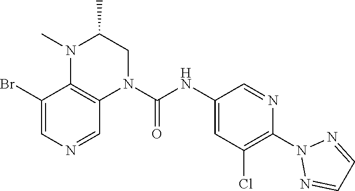 [SMILES: C[C@@H]1CN(C(=O)Nc2cnc(-n3nccn3)c(Cl)c2)c2cncc(Br)c2N1C]

To a stirred solution of Intermediate 38 (0.25 g, 1.03 mmol) and 5-chloro-6-(2H-1,2,3-triazol-2-yl)pyridin-3-amine (0.14 g, 0.72 mmol, CAS 1832583-43-3) in DCM (5 mL) at 0° C. was added triethylamine (0.43 mL, 3.09 mmol). Triphosgene (0.33 g, 1.13 mmol) was added as a solution in DCM (1 mL) and stirred at 0° C. for 15 min then at RT for 3 h. The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 20% to 45% over 7 min, held at 45% for 7.5 min, ramped to 98% over 0.1 min, held at 98% 2.4 min) to provide the title compound (28 mg). 1H NMR (400 MHz; DMSO-d₆) δ: 9.82 (br s, 1H), 8.60 (d, 1H), 8.40 (s, 1H), 8.37 (d, 1H), 8.22 (s, 1H), 8.14 (s, 2H), 3.94 (q, 1H), 3.52-3.57 (m, 2H), 3.14 (s, 3H), 1.09 (d, 3H). LCMS (Method 14): 1.62 min, 463.3 [M+H]⁺.

### Example 27: 8′—Bromo-N-(5-chloro-6-(2H-1,2,3-triazol-2-yl)pyridin-3-yl)-4,5-dihydro-2H-spiro [furan-3,2′-pyrido[4,3-b][1,4]oxazine]-4′ (3′H)-carboxamide

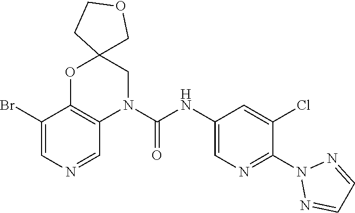 [SMILES: O=C(Nc1cnc(-n2nccn2)c(Cl)c1)N1CC2(CCOC2)Oc2c(Br)cncc21]

To a stirred solution of Intermediate 40 (0.14 g, 0.52 mmol) and 5-chloro-6-(2H-1,2,3-triazol-2-yl)pyridin-3-amine (0.18 g, 0.94 mmol, CAS 1832583-43-3) in DCM (5 mL) at 0° C. was added triethylamine (0.22 mL, 1.55 mmol). Triphosgene (0.35 g, 1.18 mmol) was added as a solution in DCM (1 mL) and stirred at 0° C. for 15 min then at RT for 3 h. The mixture was concentrated under reduced pressure. The crude product was purified by preparative HPLC (X-Select C18, 25×150 mm×5 μm, flow rate: 18 mL/min, 10 mM NH₄HCO₃ in water with MeCN, 25% to 45% over 6 min, held at 45% for 8 min, ramped to 98% over 0.1 min, held at 98% 1.9 min) to provide the title compound (24 mg). 1H NMR (400 MHz; DMSO-d₆) δ: 10.11 (br s, 1H), 8.75 (s, 1H), 8.58 (s, 1H), 8.35-8.39 (m, 2H), 8.14 (s, 2H), 3.90-4.12 (m, 4H), 3.78 (s, 2H), 2.02-2.17 (m, 2H). LCMS (Method 14): 1.65 min, 492.1 [M+H]⁺.

### Example 28:1-Bromo-N-(5-chloro-6-(2H-1,2,3-triazol-2-yl)pyridin-3-yl)-6,6a,7,8,9,10-hexahydro-5H-dipyrido[1,2-a: 3′,4′-e] pyrazine-5-carboxamide

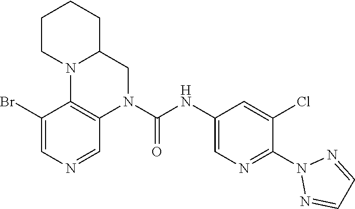 [SMILES: O=C(Nc1cnc(-n2nccn2)c(Cl)c1)N1CC2CCCCN2c2c(Br)cncc21]

To a stirred solution of Intermediate 43 (50 mg, 0.19 mmol) and 5-chloro-6-(2H-1,2,3-triazol-2-yl)pyridin-3-amine (29 mg, 0.15 mmol, CAS 1832583-43-3) in DCM (2 mL) at 0° C. was added triethylamine (0.08 mL, 0.56 mmol). Triphosgene (39 mg, 0.13 mmol) was added then the mixture was stirred at RT for 1 h. The mixture was concentrated under reduced pressure. The crude product was purified by preparative HPLC (X-Bridge Phenyl, 19×250 mm×5 μm, flow rate: 17 mL/min, 10 mM NH₄HCO₃ in water with MeCN, 30% to 60% over 9 min, held at 60% for 5 min, ramped to 100% over 0.1 min, held at 100% 2.9 min). The racemic compound was purified by chiral preparative SFC (Chiralcel-OJ-H, 30×250 mm×5 μm, flow rate: 100 g/min, 60% CO₂ with 40% MeOH: MeCN (1:1), back pressure 100 bar, Temp 30° C.)-Peak 1 isolated to provide the title compound (10 mg). 1H NMR (400 MHZ; DMSO-d₆) δ: 9.70 (s, 1H), 8.63 (s, 1H), 8.37 (d, 1H), 8.30 (s, 1H), 8.23 (s, 1H), 8.14 (s, 2H), 4.55 (d, 1H), 4.31 (dd, 1H), 3.40-3.49 (m, 1H) 3.17-3.23 (m, 1H), 2.93 (t, 1H), 1.52-1.90 (m, 5H), 1.20-1.35 (m, 1H). LCMS (Method 14): 1.50 min, 489.1 [M+H]⁺.

### Example 29:8-Bromo-N-(5-chloro-6-(2H-1,2,3-triazol-2-yl)pyridin-3-yl)-1-methyl-2,3-dihydropyrido[3,4-b]pyrazine-4 (1H)-carboxamide

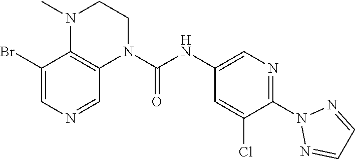 [SMILES: CN1CCN(C(=O)Nc2cnc(-n3nccn3)c(Cl)c2)c2cncc(Br)c21]

To a stirred solution of Intermediate 46 (150 mg, 0.66 mmol) and 5-chloro-6-(2H-1,2,3-triazol-2-yl)pyridin-3-amine (103 mg, 0.53 mmol, CAS 1832583-43-3) in DCM (2 mL) at 0° C. was added triethylamine (0.28 mL, 1.98 mmol) and stirred at 0° C. for 5 min. Triphosgene (137 mg, 0.46 mmol) was added then the mixture was stirred at RT for 1 h.

The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 25% to 40% over 4 min, held at 40% for 7.6 min, ramped to 98% over 0.1 min, held at 98% 2.3 min) to provide the title compound (45 mg). 1H NMR (400 MHz; DMSO-d₆) δ: 9.79 (s, 1H), 8.60 (s, 1H), 8.42 (s, 1H), 8.37 (d, 1H), 8.22 (s, 1H), 8.14 (s, 2H), 3.89 (t, 2H), 3.37 (t, 2H), 3.18 (s, 3H). LCMS (Method 14): 1.26 min, 449.1 [M+H]⁺.

### Example 30:8-Bromo-N-(5-chloro-6-(2H-1,2,3-triazol-2-yl)pyridin-3-yl)-2-methyl-2,3-dihydro-4H-pyrido[4,3-b][1,4]oxazine-4-carboxamide

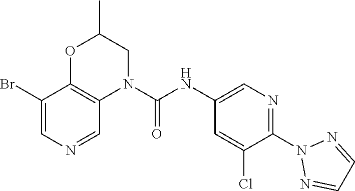 [SMILES: CC1CN(C(=O)Nc2cnc(-n3nccn3)c(Cl)c2)c2cncc(Br)c2O1]

To a stirred solution of Intermediate 49 (0.30 g, 1.31 mmol) in DCM (6 mL) at 0° C. was added triethylamine (0.54 mL, 3.93 mmol) and triphosgene (312 mg, 1.05 mmol) stirred at 0° C. for 15 min. 5-Chloro-6-(2H-1,2,3-triazol-2-yl)pyridin-3-amine (0.21 g, 1.05 mmol, CAS 1832583-43-3) was added then the mixture was stirred at RT for 2 h. The mixture was diluted with DCM and washed with brine, dried over sodium sulfate, filtered and concentrated under reduced pressure. The crude product was purified by flash column chromatography (silica gel, 230-400 mesh, eluting 60-65% EtOAc in petroleum ether) then by preparative HPLC (X-Bridge C18, 19×250 mm×5 μm, flow rate: 18 mL/min, 10 mM NH₄HCO₃ in water with MeCN, 10% to 75% over 14 min, held at 75% for 1 min) to provide the title compound (31 mg). 1H NMR (400 MHZ; DMSO-d₆) δ: 10.01 (s, 1H), 8.72 (s, 1H), 8.62 (s, 1H), 8.38 (d, 1H), 8.33 (s, 1H), 8.15 (s, 2H), 4.61 (t, 1H), 4.25 (d, 1H), 3.50 (dd, 1H), 1.41 (t, 3H). LCMS (Method 14): 1.61 min, 450.2 [M+H]⁺.

### Example 31:8-Bromo-N-(5-chloro-6-(2H-1,2,3-triazol-2-yl)pyridin-3-yl)-1,2,2-trimethyl-2,3-dihydropyrido[3,4-b]pyrazine-4 (1H)-carboxamide

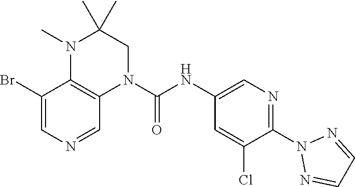 [SMILES: CN1c2c(Br)cncc2N(C(=O)Nc2cnc(-n3nccn3)c(Cl)c2)CC1(C)C]

To a stirred solution of Intermediate 53 (0.23 g, 0.89 mmol) and 5-chloro-6-(2H-1,2,3-triazol-2-yl)pyridin-3-amine (0.12 g, 0.61 mmol, CAS 1832583-43-3) in DCM (7 mL) at 0° C. was added triethylamine (0.50 mL, 3.59 mmol) and stirred at 0° C. for 15 min. Triphosgene (0.26 g, 0.89 mmol) was added as a solution in DCM (1 mL) then the mixture was stirred at RT for 2 h. The mixture was concentrated under reduced pressure. The crude product was purified by preparative HPLC (Symmetry C18, 19×300 mm×5 μm, flow rate: 18 mL/min, 10 mM NH₄HCO₃ in water with MeCN, 40% to 50% over 5 min, held at 50% for 7.6 min, ramped to 98% over 0.1 min, held at 98% for 5.3 min) to provide the title compound (24 mg). 1H NMR (400 MHZ; DMSO-d₆) δ: 9.92 (br s, 1H), 8.53 (br s, 1H), 8.44 (br s, 1H), 8.36 (d, 1H), 8.17 (s, 1H), 8.13 (s, 2H), 3.70 (s, 2H), 3.05 (s, 3H), 1.23 (s, 6H). LCMS (Method 14): 1.42 min, 477.1 [M+H]⁺.

### Example 32:8-Chloro-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide

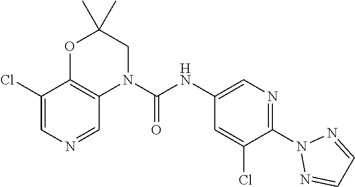 [SMILES: CC1(C)CN(C(=O)Nc2cnc(-n3nccn3)c(Cl)c2)c2cncc(Cl)c2O1]

To a stirred solution of Intermediate 56 (0.25 g, 1.26 mmol) and 5-chloro-6-(2H-1,2,3-triazol-2-yl)pyridin-3-amine (0.17 g, 0.88 mmol, CAS 1832583-43-3) in DCM (5 mL) at 0° C. was added triethylamine (0.70 mL, 5.03 mmol) and stirred at 0° C. for 15 min. Triphosgene (0.37 g, 1.26 mmol) was added as a solution in DCM (1 mL) then the mixture was stirred at RT for 3 h. The mixture was concentrated under reduced pressure. The crude product was purified by preparative HPLC (X-Select C18, 19×250 mm×5 μm, flow rate: 16 mL/min, 10 mM NH₄HCO₃ in water with MeCN, 20% to 55% over 7 min, held at 55% for 5.5 min, ramped to 98% over 0.1 min, held at 98% for 5.4 min) to provide the title compound (53 mg). 1H NMR (400 MHZ; DMSO-d₆) δ: 10.09 (br s, 1H), 8.68 (s, 1H), 8.60 (s, 1H), 8.37 (d, 1H), 8.26 (s, 1H), 8.15 (s, 2H), 3.83 (s, 2H), 1.39 (s, 6H). LCMS (Method 14): 1.76 min, 418.1 [M−H]⁻.

### Example 33:8-Bromo-N-(5-chloro-6-(2H-1,2,3-triazol-2-yl)pyridin-3-yl)-1-(tetrahydro-2H-pyran-4-yl)-2,3-dihydropyrido[3,4-b]pyrazine-4 (1H)-carboxamide

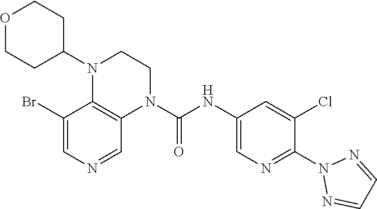 [SMILES: O=C(Nc1cnc(-n2nccn2)c(Cl)c1)N1CCN(C2CCOCC2)c2c(Br)cncc21]

To a stirred solution of Intermediate 59 (0.10 g, 0.33 mmol) and 5-chloro-6-(2H-1,2,3-triazol-2-yl)pyridin-3-amine (66 mg, 0.37 mmol, CAS 1832583-43-3) in DCM (2 mL) at 0° C. was added triethylamine (0.18 mL, 1.34 mmol) and stirred at 0° C. for 15 min. Triphosgene (50 mg, 0.16 mmol) was added as a solution in DCM (0.5 mL) then the mixture was stirred at RT for 2 h. The mixture was concentrated under reduced pressure. The crude product was purified by preparative HPLC (X-Bridge C18, 19×150 mm×5 μm, flow rate: 18 mL/min, 10 mM NH₄HCO₃ in water with MeCN, 25% to 50% over 8 min, held at 50% for 3 min, ramped to 100% over 0.1 min, held at 100% for 2.9 min) to provide the title compound (38 mg). 1H NMR (400 MHZ; DMSO-d₆) δ: 9.75 (s, 1H), 8.63 (d, 1H), 8.45 (s, 1H), 8.37 (d, 1H), 8.28 (s, 1H), 8.15 (s, 2H), 4.15-4.24 (m, 1H), 3.95 (dd, 2H), 3.79 (t, 2H), 3.37-3.40 (m, 4H), 1.86-1.91 (m, 2H), 1.67 (d, 2H). LCMS (Method 14): 1.35 min, 519.4 [M+H]⁺.

### Example 34: (R)-8-Bromo-N-(5-chloro-6-(2H-1,2,3-triazol-2-yl)pyridin-3-yl)-2-ethyl-1-methyl-2,3-dihydropyrido[3,4-b]pyrazine-4 (1H)-carboxamide

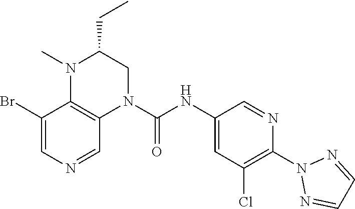 [SMILES: CC[C@@H]1CN(C(=O)Nc2cnc(-n3nccn3)c(Cl)c2)c2cncc(Br)c2N1C]

To a stirred solution of Intermediate 62 (0.10 g, 0.27 mmol) in DCM (1 mL) at 0° C. was added triethylamine (0.16 mL, 1.17 mmol) followed by triphosgene (60 mg, 0.20 mmol) and 5-chloro-6-(2H-1,2,3-triazol-2-yl)pyridin-3-amine (60 mg, 0.31 mmol, CAS 1832583-43-3) then the mixture was stirred at 0° C. for 1 h. The mixture was concentrated under reduced pressure. The crude product was purified by preparative HPLC (X-Select C18, 19×250 mm×5 μm, flow rate: 18 mL/min, 10 mM NH₄HCO₃ in water with MeCN, 50% to 80% over 7 min, held at 80% for 5 min, ramped to 98% over 0.1 min, held at 98% for 4.9 min) to provide the title compound (100 mg). 1H NMR (400 MHZ; DMSO-d₆) δ: 9.88 (s, 1H), 8.63 (d, 1H), 8.41 (s, 1H), 8.36 (d, 1H), 8.24 (s, 1H), 8.15 (s, 2H), 3.76 (dd, 1H), 3.70 (dd, 1H), 3.11 (s, 3H), 1.38-1.47 (m, 1H), 1.29-1.38 (m, 1H), 0.89 (t, 3H)-1H obscured by water signal. LCMS (Method 14): 1.47 min, 477.4 [M+H]⁺.

### Example 35: (R)-8-Bromo-N-(5-chloro-6-(2H-1,2,3-triazol-2-yl)pyridin-3-yl)-2-isopropyl-1-methyl-2,3-dihydropyrido[3,4-b]pyrazine-4 (1H)-carboxamide

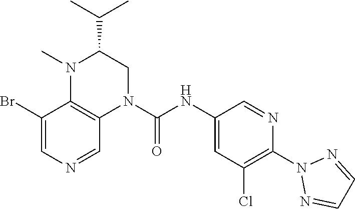 [SMILES: CC(C)[C@@H]1CN(C(=O)Nc2cnc(-n3nccn3)c(Cl)c2)c2cncc(Br)c2N1C]

To a stirred solution of Intermediate 65 (55 mg, 0.20 mmol) in DCM (1 mL) at 0° C. was added triethylamine (0.16 mL, 1.17 mmol) followed by 5-chloro-6-(2H-1,2,3-triazol-2-yl)pyridin-3-amine (28 mg, 0.20 mmol, CAS 1832583-43-3) and the mixture was stirred at 0° C. for 15 min. Triphosgene (60 mg, 0.20 mmol) was added as a solution in DCM (0.5 mL) and the mixture was stirred at RT for 3 h. The mixture was concentrated under reduced pressure then DCM (1 mL) was added followed by triethylamine (0.16 mL, 1.17 mmol) and 5-chloro-6-(2H-1,2,3-triazol-2-yl)pyridin-3-amine (28 mg, 0.20 mmol, CAS 1832583-43-3) and the mixture stirred at RT for 16 h. The mixture was concentrated under reduced pressure. The crude product was purified by preparative HPLC (Gemini NX C18, 21×250 mm×5 μm, flow rate: 18 mL/min, 10 mM NH₄HCO₃ in water with MeCN, 30% to 70% over 7 min, held at 70% for 5.5 min) to provide the title compound (31 mg). 1H NMR (400 MHZ; DMSO-d₆) δ: 10.00 (br s, 1H), 8.60 (br s, 1H), 8.44 (br s, 1H), 8.35 (d, 1H), 8.16 (s, 1H), 8.11 (s, 2H), 4.22 (br s, 1H), 3.39-3.43 (m, 1H), 3.06 (s, 3H), 2.94 (br s, 1H), 1.38-1.47 (m, 1H), 0.97 (d, 3H), 0.84 (d, 3H). LCMS (Method 14): 1.59 min, 491.4 [M+H]⁺.

### Example 36:7-Amino-8-bromo-N-(5-chloro-6-(2H-1,2,3-triazol-2-yl)pyridin-3-yl)-2,3-dihydro-4H-pyrido[4.3-b][1.4]oxazine-4-carboxamide

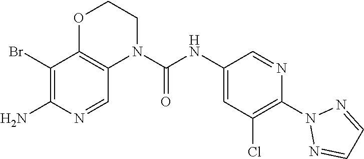 [SMILES: Nc1ncc2c(c1Br)OCCN2C(=O)Nc1cnc(-n2nccn2)c(Cl)c1]

To a stirred solution of Intermediate 69 (100 mg, 0.43 mmol) and 5-chloro-6-(2H-1,2,3-triazol-2-yl)pyridin-3-amine (85 mg, 0.43 mmol, CAS 1832583-43-3) in DCM (2 mL) at 0° C. was added triethylamine (0.15 mL, 1.09 mmol) and the mixture was stirred at 0° C. for 5 min. Triphosgene (60 mg, 0.20 mmol) was added as a solution in DCM (0.5 mL) and the mixture was stirred at RT for 16 h. The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 15% to 40% over 11 min, ramped to 98% over 0.1 min, held at 98% for 2.9 min) to provide the title compound (5 mg). 1H NMR (400 MHZ; DMSO-d₆) δ: 9.69 (br s, 1H), 8.60 (br s, 1H), 8.36 (d, 1H), 8.13 (s, 3H), 5.96 (s, 2H), 4.42 (t, 2H), 3.92 (t, 2H). LCMS (Method 14): 1.31 min, 451.3 [M+H]⁺.

### Example 37:8-Bromo-N-(5-chloro-6-(2H-1,2,3-triazol-2-yl)pyridin-3-yl)-2-(trifluoromethyl)-2,3-dihydropyrido[3,4-b]pyrazine-4 (1H)-carboxamide

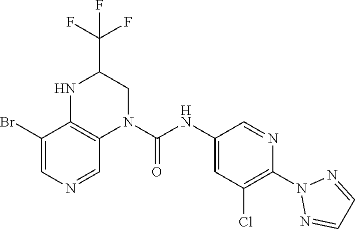 [SMILES: O=C(Nc1cnc(-n2nccn2)c(Cl)c1)N1CC(C(F)(F)F)Nc2c(Br)cncc21]

To a stirred solution of Intermediate 72 (20 mg, 0.07 mmol) in DCM (2 mL) at 0° C. was added triethylamine (0.03 mL, 0.21 mmol) and 5-chloro-6-(2H-1,2,3- triazol-2-yl)pyridin-3-amine (11 mg, 0.06 mmol, CAS 1832583-43-3) and the mixture was stirred at 0° C. for 15 min. Triphosgene (20 mg, 0.07 mmol) was added as a solution in DCM (0.5 mL) and the mixture was stirred at RT for 2 h. The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 30% to 50% over 7 min, held at 50% for 3.3 min, ramped to 98% over 0.1 min, held at 98% for 3.6 min) to provide the title compound (8 mg). 1H NMR (400 MHZ; DMSO-d₆) δ: 9.85 (br s, 1H), 8.46 (br s, 2H), 8.33 (d, 1H), 8.16 (br s, 1H), 8.11 (s, 2H), 7.25 (br s, 1H), 4.98 (br s, 1H), 4.49 (s, 1H), 3.10 (d, 1H). LCMS (Method 15): 4.47 min, 503.2 [M+H]⁺.

### Example 38:8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide

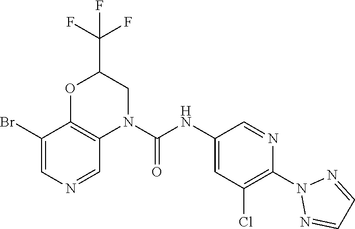 [SMILES: O=C(Nc1cnc(-n2nccn2)c(Cl)c1)N1CC(C(F)(F)F)Oc2c(Br)cncc21]

To a stirred solution of Intermediate 78 (0.10 g, 0.35 mmol) and 5-chloro-6-(2H-1,2,3-triazol-2-yl)pyridin-3-amine (69 mg, 0.35 mmol, CAS 1832583-43-3) in DCM (2 mL) at 0° C. was added triethylamine (0.30 mL, 2.10 mmol) and the mixture was stirred at 0° C. for 5 min. Triphosgene (0.10 g, 0.35 mmol) was added as a solution in DCM (0.5 mL) and the mixture was stirred at RT for 2 h. The mixture was concentrated under reduced pressure. The crude product was purified by preparative HPLC (Gemini-NX C18, 21×250 mm×5 μm, flow rate: 18 mL/min, 10 mM NH₄HCO₃ in water with MeCN, 20% to 60% over 7 min, held at 60% for 6.5 min, then to 98% over 5.5 min, held at 98% for 2.5 min) to provide the title compound (41 mg). 1H NMR (400 MHZ; DMSO-d₆) δ: 10.10 (br s, 1H), 8.76 (br s, 1H), 8.60 (br s, 1H), 8.44 (s, 1H), 8.36 (d, 1H), 8.15 (s, 2H), 5.57 (br s, 1H), 4.31 (d, 1H), 4.18 (d, 1H). LCMS (Method 14): 1.85 min, 504.3 [M+H]⁺.

### Example 39:8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide; Example 40:8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide

Example 38 (35 mg) was purified by chiral preparative SFC (Chiralcel-OJ-H, 46×250 mm×5 μm, flow rate: 100 g/min, 70% CO₂ with 30% MeOH, back pressure 100 bar, Temp 30° C.): Peak 1 provided Example 39 (13 mg) and Peak 2 provided Example 40 (13 mg).

Example 39: ¹H NMR (400 MHZ; DMSO-d₆) δ: 10.13 (br s, 1H), 8.87 (br s, 1H), 8.48 (br s, 1H), 8.36 (d, 2H), 8.12 (s, 2H), 5.50 (br s, 1H), 4.49 (br s, 1H), 4.15 (dd, 1H). LCMS (Method 14): 1.85 min, 504.3 [M+H]⁺.

Example 40: ¹H NMR (400 MHZ; DMSO-d₆) δ: 10.17 (br s, 1H), 8.93 (br s, 1H), 8.38 (d, 2H), 8.34 (br s, 1H), 8.11 (s, 2H), 5.46 (br s, 1H), 4.44 (br s, 1H), 4.13 (dd, 1H). LCMS (Method 14): 1.85 min, 504.4 [M+H]⁺.

### Example 41:8-Bromo-N-(5-chloro-6-(2H-1,2,3-triazol-2-yl)pyridin-3-yl)-7-methyl-2,3-dihydro-4H-pyrido[4,3-b][1,4]oxazine-4-carboxamide

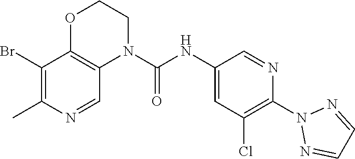 [SMILES: Cc1ncc2c(c1Br)OCCN2C(=O)Nc1cnc(-n2nccn2)c(Cl)c1]

To a stirred solution of Intermediate 83 (0.20 g, 0.75 mmol) and 5-chloro-6-(2H-1,2,3-triazol-2-yl)pyridin-3-amine (0.13 g, 0.68 mmol, CAS 1832583-43-3) in DCM (5 mL) at 0° C. was added triethylamine (0.42 mL, 3.10 mmol) and the mixture was stirred at 0° C. for 5 min. Triphosgene (0.22 g, 0.75 mmol) was added as a solution in DCM (1 mL) and the mixture was stirred at RT for 1 h. The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 30% to 50% over 7 min, held at 50% for 5 min, ramped to 98% in 0.1 min, held at 98% for 1.9 min) to provide the title compound (52 mg). 1H NMR (400 MHZ; DMSO-d₆) δ: 9.92 (br s, 1H), 8.61 (s, 2H), 8.38 (d, 1H), 8.14 (s, 2H), 4.50 (t, 2H), 3.98 (t, 2H), 2.54 (s, 3H). LCMS (Method 1): 1.36 min, 450.2 [M+H]⁺.

### Example 42:8-Bromo-N-(5-chloro-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide

### Example 43:8-Bromo-N-(5-chloro-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide

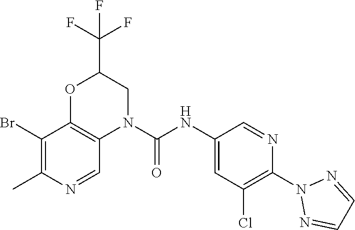 [SMILES: Cc1ncc2c(c1Br)OC(C(F)(F)F)CN2C(=O)Nc1cnc(-n2nccn2)c(Cl)c1]

To a stirred solution of Intermediate 87 (0.42 g, 1.26 mmol) and 5-chloro-6-(2H-1,2,3-triazol-2-yl)pyridin-3-amine (0.24 g, 1.26 mmol, CAS 1832583-43-3) in DCM (5 mL) at 0° C. was added triethylamine (0.53 mL, 3.78 mmol) and the mixture was stirred at 0° C. for 5 min. Triphosgene (0.37 g, 1.26 mmol) was added as a solution in DCM (1 mL) and the mixture was stirred at RT for 1 h. The mixture was concentrated under reduced pressure. The crude product was purified by preparative HPLC (X-Bridge C18, 19×250 mm×5 μm, flow rate: 17 mL/min, 10 mM NH₄HCO₃ in water with MeCN, 45% to 70% over 13 min, held at 70% for 2 min, ramped to 98% over 0.1 min, held at 98% for 1.9 min) to provide the racemic compound (94 mg). The racemic compound was purified by chiral preparative SFC (Chiralpak-AD-H, 30×250 mm×5 μm, flow rate: 100 g/min, 73% CO₂ with 27% MeOH, back pressure 100 bar, Temp 30° C.): Peak 1 provided Example 42 (36 mg) and Peak 2 provided Example 43 (32 mg).

Example 42: ¹H NMR (400 MHZ; DMSO-d₆) δ: 10.02 (br s, 1H), 8.62 (br s, 2H), 8.36 (d, 1H), 8.15 (s, 2H), 5.56 (br s, 1H), 4.21 (dd, 2H), 2.58 (s, 3H). LCMS (Method 18): 1.66 min, 520.1 [M+H]⁺.

Example 43: ¹H NMR (400 MHZ; DMSO-d₆) δ: 10.02 (br s, 1H), 8.66 (br s, 1H), 8.57 (br s, 1H), 8.37 (d, 1H), 8.14 (s, 2H), 5.54 (br s, 1H), 4.23 (dd, 2H), 2.57 (s, 3H). LCMS (Method 18): 1.66 min, 520.2 [M+H]⁺.

### Example 44:8-Bromo-N-(5-chloro-6-(2H-1,2,3-triazol-2-yl)pyridin-3-yl)-2-methyl-2,3-dihydro-4H-pyrido[4,3-b][1,4]oxazine-4-carboxamide

### Example 45:8-Bromo-N-(5-chloro-6-(2H-1,2,3-triazol-2-yl)pyridin-3-yl)-2-methyl-2,3-dihydro-4H-pyrido[4,3-b][1,4]oxazine-4-carboxamide

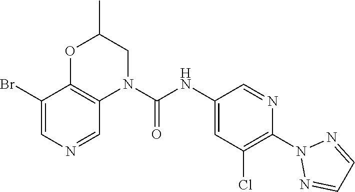 [SMILES: CC1CN(C(=O)Nc2cnc(-n3nccn3)c(Cl)c2)c2cncc(Br)c2O1]

Example 30 (25 mg) was purified by chiral preparative SFC (Chiralpak-AS-H, 30×250 mm×5 μm, flow rate: 100 g/min, 60% CO₂ with 40% MeOH, back pressure 100 bar, Temp 30° C.): Peak 1 provided Example 44 (12 mg) and Peak 2 provided Example 45 (12 mg).

Example 44: ¹H NMR (400 MHZ; DMSO-d₆) δ: 10.01 (br s, 1H), 8.71 (br s, 1H), 8.62 (br s, 1H), 8.37 (d, 1H), 8.33 (s, 1H), 8.15 (br s, 2H), 4.62 (t, 1H), 4.24 (d, 1H) 3.50 (dd, 1H), 1.40 (d, 3H). LCMS (Method 14): 1.55 min, 452.1 [M+H]⁺.

Example 45: ¹H NMR (400 MHZ; DMSO-d₆) δ: 10.01 (br s, 1H), 8.73 (br s, 1H), 8.60 (br s, 1H), 8.37 (br d, 1H), 8.32 (br s, 1H), 8.15 (br s, 2H), 4.61 (t, 1H), 4.24 (d, 1H) 3.50 (dd, 1H), 1.39 (d, 3H). LCMS (Method 14): 1.55 min, 452.1 [M+H]⁺.

### Example 46:8-Bromo-N-(5-chloro-6-(2H-1,2,3-triazol-2-yl)pyridin-3-yl)-2-ethyl-2,3-dihydro-4H-pyrido[4,3-b][1,4]oxazine-4-carboxamide

### Example 47:8-Bromo-N-(5-chloro-6-(2H-1,2,3-triazol-2-yl)pyridin-3-yl)-2-ethyl-2,3-dihydro-4H-pyrido[4,3-b][1,4]oxazine-4-carboxamide

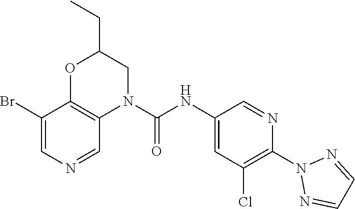 [SMILES: CCC1CN(C(=O)Nc2cnc(-n3nccn3)c(Cl)c2)c2cncc(Br)c2O1]

To a stirred solution of Intermediate 89 (0.27 g, 1.10 mmol) and 5-chloro-6-(2H-1,2,3-triazol-2-yl)pyridin-3-amine (0.34 g, 1.65 mmol, CAS 1832583-43-3) in DCM (3 mL) was added 4-nitrophenyl chloroformate (0.33 g, 1.65 mmol) and stirred for 15 min at RT. The mixture was cooled at 0° C. then triethylamine (0.46 mL, 3.30 mmol) was added dropwise and the mixture was stirred at RT for 5 h. The mixture was diluted with water and extracted with EtOAc. The organics were washed with water, dried over Na₂SO₄, filtered and concentrated under reduced pressure. The crude product was purified by preparative HPLC (X-Bridge Phenyl, 19×250 mm×5 μm, flow rate: 17 mL/min, 10 mM NH₄HCO₃ in water with MeCN, 30% to 60% over 10 min, then to 65% over 3.5 min, ramped to 100% over 0.1 min, held at 100% for 2.4 min) to provide the racemic compound (310 mg). The racemic compound was purified by chiral preparative SFC (Chiralpak-AS-H, 30×250 mm×5 μm, flow rate: 100 g/min, 70% CO₂ with 30% MeOH, back pressure 100 bar, Temp 30° C.): Peak 1 provided Example 46 (127 mg) and Peak 2 provided Example 47 (150 mg).

Example 46: ¹H NMR (400 MHZ; DMSO-d₆) δ: 10.00 (br s, 1H), 8.69 (br s, 1H), 8.64 (d, 1H), 8.37 (d, 1H), 8.35 (s, 1H), 8.16 (s, 2H), 4.41-4.47 (m, 1H), 4.18-4.24 (m, 1H), 3.59 (dd, 1H), 1.75-1.66 (m, 2H), 1.05 (t, 3H). LCMS (Method 14): 1.72 min, 466.2 [M+H]⁺. Example 47: ¹H NMR (400 MHZ; DMSO-d₆) δ: 10.00 (br s, 1H), 8.69 (br s, 1H), 8.63 (d, 1H), 8.37 (d, 1H), 8.35 (s, 1H), 8.16 (s, 2H), 4.41-4.47 (m, 1H), 4.18-4.24 (m, 1H), 3.59 (dd, 1H), 1.75-1.66 (m, 2H), 1.05 (t, 3H). LCMS (Method 14): 1.72 min, 466.2 [M+H]⁺.

### Example 48:8-Bromo-N-(5-chloro-6-(2H-1,2,3-triazol-2-yl)pyridin-3-yl)-2-isopropyl-2,3-dihydro-4H-pyrido[4,3-b][1,4]oxazine-4-carboxamide

### Example 49:8-Bromo-N-(5-chloro-6-(2H-1,2,3-triazol-2-yl)pyridin-3-yl)-2-isopropyl-2,3-dihydro-4H-pyrido[4,3-b][1,4]oxazine-4-carboxamide

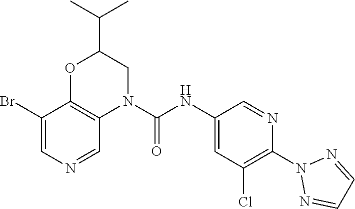 [SMILES: CC(C)C1CN(C(=O)Nc2cnc(-n3nccn3)c(Cl)c2)c2cncc(Br)c2O1]

To a stirred solution of Intermediate 92 (0.30 g, 0.99 mmol) and 5-chloro-6-(2H-1,2,3-triazol-2-yl)pyridin-3-amine (0.21 g 0.99 mmol, CAS 1832583-43-3) in DCM (10 mL) at 0° C. was added triethylamine (0.55 mL, 3.97 mmol) and the mixture was stirred at 0° C. for 5 min. Triphosgene (0.21 g, 0.69 mmol) was added as a solution in DCM (1 mL) and the mixture was stirred at RT for 2 h. The mixture was concentrated under reduced pressure. The crude product was purified by preparative HPLC (X-Bridge C18, 19×250 mm×5 μm, flow rate: 18 mL/min, 10 mM NH₄HCO₃ in water with MeCN, 35% to 60% over 9 min, held at 60% for 2 min, ramped to 100% over 0.1 min, held at 100% for 3.9 min) to provide the racemic compound (120 mg). The racemic compound was purified by chiral preparative SFC (Chiralpak-AD-H, 30×250 mm×5 μm, flow rate: 100 g/min, 60% CO₂ with 40% MeOH, back pressure 100 bar, Temp 30° C.): Peak 1 provided Example 48 (32 mg) and Peak 2 provided Example 49 (40 mg).

Example 48: ¹H NMR (400 MHZ; DMSO-d₆) δ: 9.98 (br s, 1H), 8.70 (br s, 1H), 8.62 (br s, 1H), 8.39 (d, 1H), 8.35 (s, 1H), 8.15 (s, 2H), 4.18-4.28 (m, 2H), 3.59 (dd, 1H), 1.90-1.98 (m, 1H), 1.08 (d, 3H), 1.02 (d, 3H). LCMS (Method 18): 1.65 min, 480.2 [M+H]⁺.

Example 49: ¹H NMR (400 MHZ; DMSO-d₆) δ: 9.99 (br s, 1H), 8.70 (br s, 1H), 8.62 (br s, 1H), 8.39 (d, 1H), 8.34 (s, 1H), 8.15 (s, 2H), 4.18-4.28 (m, 2H), 3.59 (dd, 1H), 1.90-1.98 (m, 1H), 1.07 (d, 3H), 1.03 (d, 3H). LCMS (Method 18): 1.65 min, 480.2 [M+H]⁺.

### Example 50:8-Bromo-N-(5-chloro-6-(2H-1,2,3-triazol-2-yl)pyridin-3-yl)-2-cyclopropyl-2,3-dihydro-4H-pyrido[4,3-b][1,4]oxazine-4-carboxamide

### Example 51:8-Bromo-N-(5-chloro-6-(2H-1,2,3-triazol-2-yl)pyridin-3-yl)-2-cyclopropyl-2,3-dihydro-4H-pyrido[4,3-b][1,4]oxazine-4-carboxamide

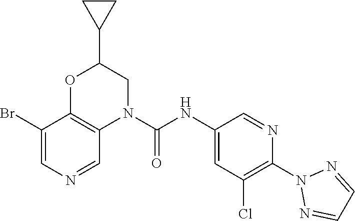 [SMILES: O=C(Nc1cnc(-n2nccn2)c(Cl)c1)N1CC(C2CC2)Oc2c(Br)cncc21]

To a stirred solution of Intermediate 95 (0.10 g, 0.33 mmol) and 5-chloro-6-(2H-1,2,3-triazol-2-yl)pyridin-3-amine (0.10 g, 0.50 mmol, CAS 1832583-43-3) in DCM (2 mL) was added 4-nitrophenyl chloroformate (67 mg, 0.33 mmol) and stirred for 30 min at RT. The mixture was cooled at 0° C. then triethylamine (0.28 mL, 1.99 mmol) was added dropwise and the mixture was stirred at RT for 4 h. The mixture was diluted with water and extracted with EtOAc. The organics were washed with water, dried over Na₂SO₄, filtered and concentrated under reduced pressure. The crude product was purified by preparative HPLC (X-Bridge C18, 19×250 mm×5 μm, flow rate: 17 mL/min, 10 mM NH₄HCO₃ in water with MeCN, 20% to 60% over 8 min, then to 98% over 2.1 min, held at 98% for 1.9 min) to provide the racemic compound (65 mg). The racemic compound was purified by chiral preparative SFC (Chiralpak-AS-H, 30×250 mm×5 μm, flow rate: 100 g/min, 60% CO₂ with 40% MeOH, back pressure 100 bar, Temp 30° C.): Peak 1 provided Example 50 (24 mg) and Peak 2 provided Example 51 (24 mg).

Example 50: ¹H NMR (400 MHz; DMSO-d₆) δ: 10.00 (br s, 1H), 8.69 (br s, 1H), 8.64 (br d, 1H), 8.37 (d, 1H), 8.34 (s, 1H), 8.16 (s, 2H), 4.22 (d, 1H), 3.91-3.98 (m, 1H), 3.72 (dd, 1H), 1.15-1.06 (m, 1H), 0.68-0.48 (m, 4H). LCMS (Method 14): 1.77 min, 478.2 [M+H]⁺.

Example 51: ¹H NMR (400 MHZ; DMSO-d₆) δ: 10.00 (br s, 1H), 8.69 (br s, 1H), 8.64 (br d, 1H), 8.37 (d, 1H), 8.34 (s, 1H), 8.15 (s, 2H), 4.22 (d, 1H), 3.91-3.98 (m, 1H), 3.72 (dd, 1H), 1.15-1.06 (m, 1H), 0.68-0.48 (m, 4H). LCMS (Method 14): 1.77 min, 478.2 [M+H]⁺.

### Example 52: N-(5-Chloro-6-(2H-1,2,3-triazol-2-yl)pyridin-3-yl)-8-cyano-7-methyl-2-(trifluoromethyl)-2,3-dihydro-4H-pyrido[4,3-b][1,4]oxazine-4-carboxamide

### Example 53: N-(5-Chloro-6-(2H-1,2,3-triazol-2-yl)pyridin-3-yl)-8-cyano-7-methyl-2-(trifluoromethyl)-2,3-dihydro-4H-pyrido[4,3-b][1,4]oxazine-4-carboxamide

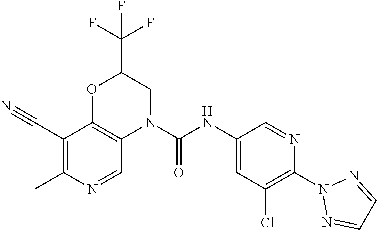 [SMILES: Cc1ncc2c(c1C#N)OC(C(F)(F)F)CN2C(=O)Nc1cnc(-n2nccn2)c(Cl)c1]

To a stirred solution of Intermediate 97 (0.10 g, 0.37 mmol) and 5-chloro-6-(2H-1,2,3-triazol-2-yl)pyridin-3-amine (0.12 g, 0.56 mmol, CAS 1832583-43-3) in DCM (1 mL) was added 4-nitrophenyl chloroformate (0.11 g, 0.56 mmol) and stirred for 30 min at RT. The mixture was cooled at 0° C. then triethylamine (0.15 mL, 1.11 mmol) was added dropwise and the mixture was stirred at RT for 5 h. The mixture was diluted with water and extracted with EtOAc. The organics were washed with water, dried over Na₂SO₄, filtered and concentrated under reduced pressure. The crude product was purified by preparative HPLC (X-Bridge C18, 19×250 mm×5 μm, flow rate: 17 mL/min, 10 mM NH₄HCO₃ in water with MeCN, 40% to 45% over 8 min, held at 45% for 3 min, ramped to 98% over 0.1 min, held at 98% for 1.9 min) to provide the racemic compound (54 mg). The racemic compound was purified by chiral preparative SFC (Chiralcel-OJ-H, 30×250 mm×5 μm, flow rate: 100 g/min, 65% CO₂ with 35% MeOH, back pressure 100 bar, Temp 30° C.): Peak 1 provided Example 52 (15 mg) and Peak 2 provided Example 53 (15 mg).

Example 52: ¹H NMR (400 MHZ; DMSO-d₆) δ: 10.09 (br s, 1H), 8.89 (br s, 1H), 8.63 (br s, 1H), 8.36 (d, 1H), 8.16 (s, 2H), 5.71-5.63 (m, 1H), 4.35-4.20 (m, 2H), 2.64 (s, 3H). LCMS (Method 14): 1.77 min, 465.2 [M+H]⁺.

Example 53: ¹H NMR (400 MHZ; DMSO-d₆) δ: 10.10 (br s, 1H), 8.90 (br s, 1H), 8.62 (br s, 1H), 8.36 (d, 1H), 8.16 (s, 2H), 5.71-5.63 (m, 1H), 4.35-4.20 (m, 2H), 2.64 (s, 3H). LCMS (Method 14): 1.77 min, 465.2 [M+H]⁺.

### Example 54: N-(5-Chloro-6-(2H-1,2,3-triazol-2-yl)pyridin-3-yl)-8-cyano-2-(trifluoromethyl)-2,3-dihydro-4H-pyrido[4,3-b][1,4]oxazine-4-carboxamide

### Example 55: N-(5-Chloro-6-(2H-1,2,3-triazol-2-yl)pyridin-3-yl)-8-cyano-2-(trifluoromethyl)-2,3-dihydro-4H-pyrido[4,3-b][1,4]oxazine-4-carboxamide

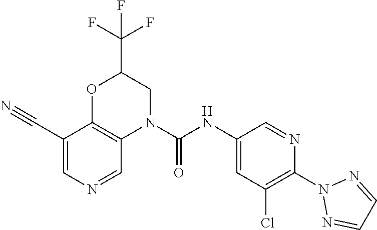 [SMILES: N#Cc1cncc2c1OC(C(F)(F)F)CN2C(=O)Nc1cnc(-n2nccn2)c(Cl)c1]

To a stirred solution of Intermediate 99 (0.16 g, 0.57 mmol) and 5-chloro-6-(2H-1,2,3-triazol-2-yl)pyridin-3-amine (0.17 g, 0.86 mmol, CAS 1832583-43-3) in DCM (2 mL) was added 4-nitrophenyl chloroformate (0.17 g, 0.86 mmol) and stirred for 5 min at RT. The mixture was cooled at 0° C. then triethylamine (0.24 mL, 1.72 mmol) was added dropwise and the mixture was stirred at RT for 5 h. The mixture was diluted with water and extracted with EtOAc. The organics were washed with water, dried over Na₂SO₄, filtered and concentrated under reduced pressure. The crude product was purified by preparative HPLC (X-Bridge C18, 19×150 mm×5 μm, flow rate: 17 mL/min, 10 mM NH₄HCO₃ in water with MeCN, 40% to 45% over 8 min, held at 45% for 3 min, ramped to 98% over 0.1 min, held at 98% for 1.9 min) to provide the racemic compound (95 mg). The racemic compound was purified by chiral preparative SFC (Chiralpak-IJ-H, 30×250 mm×5 μm, flow rate: 100 g/min, 70% CO₂ with 30% MeOH, back pressure 100 bar, Temp 30° C.): Peak 1 provided Example 54 (15 mg) and Peak 2 provided Example 55 (15 mg).

Example 54: ¹H NMR (400 MHZ; DMSO-d₆) δ: 10.18 (br s, 1H), 9.04 (br s, 1H), 8.72 (br s, 1H), 8.62 (br s, 1H), 8.36 (d, 1H), 8.16 (s, 2H), 5.71-5.63 (m, 1H), 4.39-4.20 (m, 2H). LCMS (Method 18): 1.73 min, 451.2 [M+H]⁺.

Example 55: ¹H NMR (400 MHZ; DMSO-d₆) δ: 10.18 (br s, 1H), 9.05 (br s, 1H), 8.71 (br s, 1H), 8.62 (br s, 1H), 8.36 (d, 1H), 8.16 (s, 2H), 5.71-5.63 (m, 1H), 4.39-4.20 (m, 2H). LCMS (Method 18): 1.73 min, 451.2 [M+H]⁺.

### Example 56: N-(5-Chloro-6-(2H-1,2,3-triazol-2-yl)pyridin-3-yl)-8-methoxy-2-(trifluoromethyl)-2,3-dihydro-4H-pyrido[4,3-b][1,4]oxazine-4-carboxamide

### Example 57: N-(5-Chloro-6-(2H-1,2,3-triazol-2-yl)pyridin-3-yl)-8-methoxy-2-(trifluoromethyl)-2,3-dihydro-4H-pyrido[4,3-b][1,4]oxazine-4-carboxamide

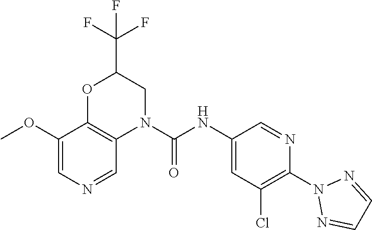 [SMILES: COc1cncc2c1OC(C(F)(F)F)CN2C(=O)Nc1cnc(-n2nccn2)c(Cl)c1]

To a stirred solution of Intermediate 101 (0.18 g, 0.46 mmol) and 5-chloro-6-(2H-1,2,3-triazol-2-yl)pyridin-3-amine (96 mg, 0.46 mmol, CAS 1832583-43-3) in DCM (2 mL) was added 4-nitrophenyl chloroformate (93 mg, 0.46 mmol) and stirred for 15 min at RT. The mixture was cooled at 0° C. then triethylamine (0.19 mL, 1.38 mmol) was added dropwise and the mixture was stirred at RT for 5 h. The mixture was diluted with water and extracted with EtOAc. The organics were washed with water, dried over Na₂SO₄, filtered and concentrated under reduced pressure. The crude product was purified by preparative HPLC (X-Bridge Phenyl, 19×250 mm×5 μm, flow rate: 17 mL/min, 10 mM NH₄HCO₃ in water with MeCN, 20% to 50% over 9 min, held at 50% for 4 min, ramped to 100% over 0.1 min, held at 100% for 2.9 min) to provide the racemic compound (240 mg). The racemic compound was purified by chiral preparative SFC (Chiralpak-IC, 30×250 mm×5 μm, flow rate: 100 g/min, 60% CO₂ with 40% MeOH, back pressure 100 bar, Temp 30° C.): Peak 1 provided Example 56 (46 mg) and Peak 2 provided Example 57 (53 mg).

Example 56: ¹H NMR (400 MHZ; DMSO-d₆) δ: 10.04 (br s, 1H), 8.65 (br d, 1H), 8.42 (br s, 1H), 8.36 (d, 1H), 8.16 (s, 2H), 8.12 (s, 1H), 5.49-5.42 (m, 1H), 4.23-4.10 (m, 2H), 3.93 (s, 3H). LCMS (Method 14): 1.41 min, 456.2 [M+H]⁺.

Example 57: ¹H NMR (400 MHZ; DMSO-d₆) δ: 10.04 (br s, 1H), 8.65 (br d, 1H), 8.42 (br s, 1H), 8.36 (d, 1H), 8.16 (s, 2H), 8.12 (s, 1H), 5.49-5.42 (m, 1H), 4.23-4.10 (m, 2H), 3.93 (s, 3H). LCMS (Method 14): 1.41 min, 456.2 [M+H]⁺.

### Example 58:5-Bromo-N-(5-chloro-6-(2H-1,2,3-triazol-2-yl)pyridin-3-yl)-3-cyano-3,4-dihydro-1,7-naphthyridine-1 (2H)-carboxamide

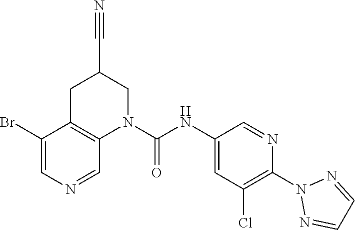 [SMILES: N#CC1Cc2c(Br)cncc2N(C(=O)Nc2cnc(-n3nccn3)c(Cl)c2)C1]

To a stirred solution of Intermediate 104 (55 mg, 0.22 mmol) and 5-chloro-6-(2H-1,2,3-triazol-2-yl)pyridin-3-amine (44 mg, 0.22 mmol, CAS 1832583-43-3) in DCM (2.8 mL) at 0° C. was added triethylamine (0.09 mL, 0.64 mmol) and the mixture was stirred at 0° C. for 5 min. Triphosgene (70 mg, 0.24 mmol) was added as a solution in DCM (1 mL) and the mixture was stirred at RT for 3 h. The mixture was concentrated under reduced pressure. The crude product was purified by preparative HPLC (X-Select C18, 19×250 mm×5 μm, flow rate: 18 mL/min, 10 mM NH₄HCO₃ in water with MeCN, 30% to 45% over 8 min, held at 45% for 2.2 min, ramped to 100% in 0.2 min) to provide the title compound (13 mg). 1H NMR (400 MHz; DMSO-d₆) δ: 10.16 (br s, 1H), 8.72 (br s, 1H), 8.58 (br s, 1H), 8.47 (br s, 1H), 8.34 (d, 1H), 8.15 (s, 2H), 4.30 (dd, 1H), 3.94 (d, 1H), 3.72-3.66 (m, 1H), 3.21 (dd, 1H), 3.07 (dd, 1H). LCMS (Method 14): 1.55 min, 461.1 [M+H]⁺.

### Example 59:8-Cyano-7-methyl-2-(trifluoromethyl)-N-(2-(trifluoromethyl)pyridin-4-yl)-2,3-dihydro-4H-pyrido[4,3-b][1,4]oxazine-4-carboxamide

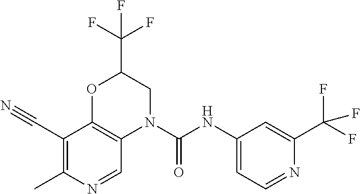 [SMILES: Cc1ncc2c(c1C#N)OC(C(F)(F)F)CN2C(=O)Nc1ccnc(C(F)(F)F)c1]

To a stirred solution of Intermediate 97 (0.10 g, 0.36 mmol) and 2-(trifluoromethyl)pyridin-4-amine (70 mg, 0.43 mmol, CAS 147149-98-2) in DCM (2.8 mL) at 0° C. was added triethylamine (0.24 mL, 1.73 mmol) and the mixture was stirred at 0° C. for 15 min. Triphosgene (137 mg, 0.43 mmol) was added as a solution in DCM (1 mL) and the mixture was stirred at RT for 5 h. The mixture was diluted with water and extracted with EtOAc. The combined organics were washed with water, brine, dried over anhydrous Na₂SO₄, filtered and concentrated under reduced pressure. The crude product was purified by flash column chromatography (silica gel, 230-400 mesh, eluting 0-30% ethyl acetate in hexane) to provide the racemic compound (78 mg). The racemic compound was purified by chiral preparative SFC (Lux Cellulose, 30×250 mm×5 μm, flow rate: 100 g/min, 80% CO₂ with 20% MeOH, back pressure 100 bar, Temp 30° C.): Peak 2 provided Example 59 (29 mg). 1H NMR (400 MHz; DMSO-d₆) δ: 10.14 (br s, 1H), 8.83 (s, 1H), 8.58 (d, 1H), 7.98 (d, 1H), 7.72 (d, 1H), 5.69-5.61 (m, 1H), 4.28-4.20 (m, 2H), 2.63 (s, 3H). LCMS (Method 14): 1.80 min, 432.3 [M+H]⁺.

### Example 60: N-(5-Chloro-6-(difluoromethoxy)pyridin-3-yl)-8-cyano-7-methyl-2-(trifluoromethyl)-2,3-dihydro-4H-pyrido[4,3-b][1,4]oxazine-4-carboxamide

### Example 61: N-(5-Chloro-6-(difluoromethoxy)pyridin-3-yl)-8-cyano-7-methyl-2-(trifluoromethyl)-2,3-dihydro-4H-pyrido[4,3-b][1,4]oxazine-4-carboxamide

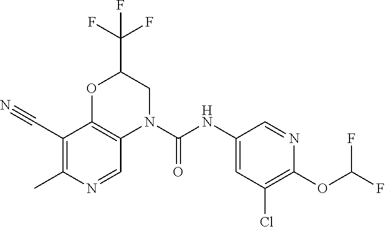 [SMILES: Cc1ncc2c(c1C#N)OC(C(F)(F)F)CN2C(=O)Nc1cnc(OC(F)F)c(Cl)c1]

To a stirred solution of Intermediate 97 (0.12 g, 0.38 mmol) and 5-chloro-6-(difluoromethoxy)pyridin-3-amine (92 mg, 0.45 mmol, CAS 1832583-48-8) in DCM (1.2 mL) at 0° C. was added triethylamine (0.16 mL, 1.13 mmol) and the mixture was stirred at 0° C. for 5 min. Triphosgene (0.11 g, 0.38 mmol) was added as a solution in DCM (1 mL) and the mixture was stirred at RT for 1 h. The mixture was diluted with water and extracted with EtOAc. The combined organics were washed with water, brine, dried over anhydrous Na₂SO₄, filtered and concentrated under reduced pressure. The crude product was purified by flash column chromatography (silica gel, 230-400 mesh, eluting 0-100% ethyl acetate in petroleum ether) to provide the racemic compound (45 mg). The racemic compound was purified by chiral preparative SFC (Lux Cellulose, 30×250 mm×5 μm, flow rate: 100 g/min, 75% CO₂ with 25% MeOH, back pressure 100 bar, Temp 30° C.): Peak 1 provided Example 60 (11 mg) and Peak 2 provided Example 61 (11 mg).

Example 60: ¹H NMR (400 MHZ; DMSO-d₆) δ: 9.74 (br s, 1H), 8.85 (br s, 1H), 8.24 (br s, 1H), 8.19 (br d, 1H), 7.67 (t, 1H), 5.68-5.59 (m, 1H), 4.28-4.18 (m, 2H), 2.62 (s, 3H). LCMS (Method 14): 1.98 min, 464.2 [M+H]⁺.

Example 61: ¹H NMR (400 MHZ; DMSO-d₆) δ: 9.76 (br s, 1H), 8.86 (br s, 1H), 8.24 (br s, 1H), 8.19 (d, 1H), 7.67 (t, 1H), 5.68-5.58 (m, 1H), 4.29-4.18 (m, 2H), 2.62 (s, 3H). LCMS (Method 14): 1.98 min, 464.2 [M+H]⁺.

### Example 62:8-Cyano-N-(5-cyano-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide

### Example 63:8-Cyano-N-(5-cyano-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide

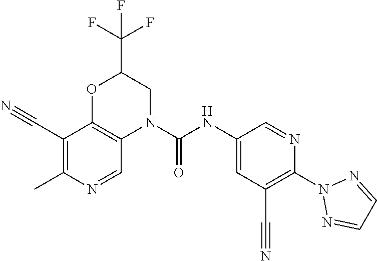 [SMILES: Cc1ncc2c(c1C#N)OC(C(F)(F)F)CN2C(=O)Nc1cnc(-n2nccn2)c(C#N)c1]

To a stirred solution of Intermediate 97 (0.11 g, 0.37 mmol) and 5-amino-2-(triazol-2-yl)pyridine-3-carbonitrile (0.10 g, 0.46 mmol, CAS 2097854-16-3) in DCM (1.7 mL) at 0° C. was added triethylamine (0.19 mL, 1.37 mmol) and the mixture was stirred at 0° C. for 5 min. Triphosgene (0.11 g, 0.38 mmol) was added as a solution in DCM (1.7 mL) and the mixture was stirred at RT for 1 h. The mixture was diluted with water and extracted with DCM. The combined organics were washed with water, brine, dried over anhydrous Na₂SO₄, filtered and concentrated under reduced pressure. The crude product was purified by flash column chromatography (silica gel, 230-400 mesh, eluting 0-100% ethyl acetate in petroleum ether) to provide the racemic compound (48 mg). The racemic compound was purified by chiral preparative SFC (Chiralpak IJ, 30×250 mm×5 μm, flow rate: 100 g/min, 60% CO₂ with 40% MeOH, back pressure 100 bar, Temp 30° C.): Peak 1 provided Example 62 (12 mg) and Peak 2 provided Example 63 (15 mg).

Example 62: ¹H NMR (400 MHZ; DMSO-d₆) δ: 10.19 (br s, 1H), 8.95 (br s, 1H), 8.82 (br s, 1H), 8.57 (d, 1H), 8.27 (s, 2H), 5.63 (br s, 1H), 4.35-4.30 (m, 1H), 4.23 (dd, 1H), 2.63 (s, 3H). LCMS (Method 14): 1.63 min, 456.3 [M+H]⁺.

Example 63: ¹H NMR (400 MHZ; DMSO-d₆) δ: 10.16 (br s, 1H), 8.90-8.87 (m, 2H), 8.57 (d, 1H), 8.29 (s, 2H), 5.69-5.65 (m, 1H), 4.33-4.21 (m, 2H), 2.64 (s, 3H). LCMS (Method 14): 1.63 min, 456.3 [M+H]⁺.

### Example 64: N-(6-(2H-1,2,3-triazol-2-yl)-5-(trifluoromethyl)pyridin-3-yl)-8-cyano-7-methyl-2-(trifluoromethyl)-2,3-dihydro-4H-pyrido[4,3-b][1,4]oxazine-4-carboxamide

### Example 65: N-(6-(2H-1,2,3-triazol-2-yl)-5-(trifluoromethyl)pyridin-3-yl)-8-cyano-7-methyl-2-(trifluoromethyl)-2,3-dihydro-4H-pyrido[4,3-b][1,4]oxazine-4-carboxamide

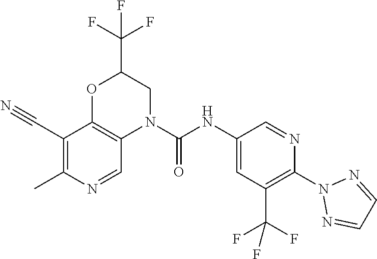 [SMILES: Cc1ncc2c(c1C#N)OC(C(F)(F)F)CN2C(=O)Nc1cnc(-n2nccn2)c(C(F)(F)F)c1]

To a stirred solution of Intermediate 97 (0.15 g, 0.57 mmol) and 6-(2H-1,2,3-triazol-2-yl)-5-(trifluoromethyl)pyridin-3-amine (0.14 g, 0.63 mmol, CAS 1832582-59-8) in DCM (10 mL) at 0° C. was added triethylamine (0.24 mL, 1.72 mmol) and the mixture was stirred at 0° C. for 5 min. Triphosgene (0.17 g, 0.57 mmol) was added as a solution in DCM (3 mL) and the mixture was stirred at RT for 1 h. The mixture was diluted with water and extracted with DCM. The combined organics were washed with water, brine, dried over anhydrous Na₂SO₄, filtered and concentrated under reduced pressure. The crude product was purified by flash column chromatography (silica gel, 230-400 mesh, eluting 0-50% ethyl acetate in petroleum ether) to provide the racemic compound (90 mg). The racemic compound was purified by chiral preparative SFC (Chiralpak-IJ, 30×250 mm×5 μm, flow rate: 100 g/min, 80% CO₂ with 20% MeOH, back pressure 100 bar, Temp 30° C.): Peak 1 provided Example 64 (28 mg) and Peak 2 provided Example 65 (28 mg).

Example 64: ¹H NMR (400 MHZ; DMSO-d₆) δ: 10.23 (br s, 1H), 8.93 (br s, 2H), 8.58 (d, 1H), 8.17 (s, 2H), 5.66 (br s, 1H), 4.38-4.20 (m, 2H), 2.64 (s, 3H). LCMS (Method 14): 1.83 min, 499.3 [M+H]⁺.

Example 65: ¹H NMR (400 MHZ; DMSO-d₆) δ: 10.23 (br s, 1H), 8.93 (br s, 2H), 8.58 (d, 1H), 8.17 (s, 2H), 5.65 (br s, 1H), 4.38-4.21 (m, 2H), 2.64 (s, 3H). LCMS (Method 14): 1.83 min, 499.3 [M+H]⁺.

### Example 66:8-Cyano-N-(6-(cyclopropyl(methyl) carbamoyl)-5-(difluoromethyl)pyridin-3-yl)-7-methyl-2-(trifluoromethyl)-2,3-dihydro-4H-pyrido[4,3-b][1,4]oxazine-4-carboxamide

### Example 67:8-Cyano-N-(6-(cyclopropyl(methyl) carbamoyl)-5-(difluoromethyl)pyridin-3-yl)-7-methyl-2-(trifluoromethyl)-2,3-dihydro-4H-pyrido[4,3-b][1,4]oxazine-4-carboxamide

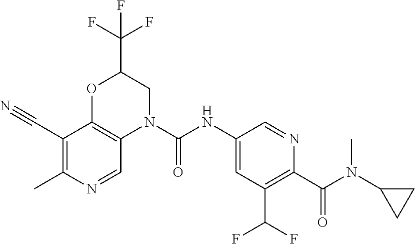 [SMILES: Cc1ncc2c(c1C#N)OC(C(F)(F)F)CN2C(=O)Nc1cnc(C(=O)N(C)C2CC2)c(C(F)F)c1]

To a stirred solution of Intermediate 97 (70 mg, 0.28 mmol) and Intermediate 107 (70 mg, 0.29 mmol) in THF (5 mL) at 0° C. was added DIPEA (0.12 mL, 0.70 mmol) and the mixture was stirred at 0° C. for 5 min. Triphosgene (69 mg, 0.23 mmol) was added as a solution in DCM (1 mL) and the mixture was stirred at 60° C. for 1 h. The mixture was diluted with water and extracted with EtOAc. The combined organics were washed with water, brine, dried over anhydrous Na₂SO₄, filtered and concentrated under reduced pressure. The crude product was purified by flash column chromatography (silica gel, 230-400 mesh, eluting 0-100% ethyl acetate in petroleum ether) to provide the racemic compound (80 mg). The racemic compound was purified by chiral preparative SFC (Chiralpak-IK, 30×250 mm×5 μm, flow rate: 100 g/min, 75% CO₂ with 25% MeOH, back pressure 100 bar, Temp 30° C.): Peak 1 provided Example 66 (18 mg) and Peak 2 provided Example 67 (20 mg).

Example 66: ¹H NMR (400 MHZ; DMSO-d₆) δ: 9.92 (br s, 1H), 8.89 (br s, 1H), 8.79 (br s, 1H), 8.25 (s, 1H), 7.11 (t, 1H), 5.63 (br s, 1H), 4.38-4.15 (m, 2H), 3.01 (s, 3H), 2.83 (d, 1H), 2.62 (s, 3H), 0.46-0.37 (m, 4H). LCMS (Method 14): 1.73 min, 511.4 [M+H]⁺ Example 67: ¹H NMR (400 MHZ; DMSO-d₆) δ: 9.92 (br s, 1H), 8.91 (br s, 1H), 8.77 (br s, 1H), 8.25 (s, 1H), 7.10 (t, 1H), 5.62 (br s, 1H), 4.38-4.15 (m, 2H), 3.01 (s, 3H), 2.83 (d, 1H), 2.62 (s, 3H), 0.46-0.37 (m, 4H). LCMS (Method 14): 1.73 min, 511.4 [M+H]⁺.

### Biological Assays

### MALT1 Protease Assay 1 (Assay 1)

MALT1 protease activity was assessed in vitro by measuring the cleavage of a fluorogenic tetrapeptide substrate.

A protein (MALT1-GS-Ub) comprising 6-His-hMALT-1 (residues 339-715), fused to ubiquitin with an 8× GGS linker, was expressed in E. coli, and purified using chitin resin to remove contaminants followed by affinity chromatography purification and size-exclusion chromatography, according to standard protocols.

Briefly, MALT1-GS-Ub (300-600 nM) was incubated with substrate (Ac-LVSR-AMC, 100 μM) in reaction buffer comprising 50 mM HEPES (pH7.0), 25 mM KCl, 0.1% (v/v) CHAPS and 1 mM TCEP.

Test compounds dissolved in DMSO were dispensed into assay plates (384-well, black, shallow ProxiPlates). 7 μl enzyme solution was added and incubated at room temperature for 30 minutes to allow compound binding to occur. 2 μl substrate solution was then added and the fluorescence (excitation 360 nm, emission 460 nm) read every 15 minutes using a suitable plate reader. Final assay DMSO concentration was 1%. Linearity over 60 minutes was confirmed, and assay signal was calculated by subtracting raw counts at time 0 from those at 60 minutes. % inhibition was calculated for each well, using no enzyme for 100% inhibition controls and no compound for 0% inhibition controls. IC₅₀ values were calculated using GraphPad Prism using a 4-parameter non-linear regression curve fit.

### MALT1 Protease Assay 2 (Assay 2)

MALT1 protease activity was assessed in vitro by measuring the cleavage of a fluorogenic tetrapeptide substrate.

For a higher sensitivity assay, commercially available FL hMALT1 enzyme (Abcam, 1-10 nM) was incubated with substrate (Ac-LVSR-AMC, 100 μM) in reaction buffer comprising 50 mM HEPES (pH7.0), 25 mM KCl, 0.1% (v/v) CHAPS, 1 mM TCEP and 0.7 M sodium citrate.

Test compounds dissolved in DMSO were dispensed into assay plates (384-well, black, shallow ProxiPlates). 7 μl enzyme solution was added and incubated at room temperature for 30 minutes to allow compound binding to occur. 2 μl substrate solution was then added and the fluorescence (excitation 360 nm, emission 460 nm) read every 15 minutes using a suitable plate reader. Final assay DMSO concentration was 1%. Linearity over 60 minutes was confirmed, and assay signal was calculated by subtracting raw counts at time 0 from those at 60 minutes. % inhibition was calculated for each well, using no enzyme for 100% inhibition controls and no compound for 0% inhibition controls. IC₅₀ values were calculated using GraphPad Prism using a 4-parameter non-linear regression curve fit.

### Human IL-2 Release Phenotypic Assay (Assay 3)

This assay measured the inhibition by MALT1 inhibitors of the IL-2 release from stimulated Jurkat cells, a human immortalized T lymphocyte cell line. IL-2 production from Jurkat cells is regulated by activation of NFκB signalling, which is in turn regulated by MALT1 protease activity.

Jurkat cells (clone E6-1, ATCC) were cultured in RPMI1640 supplemented with 10% (v/v) FBS and 1% (v/v) penicillin/streptomycin (100× liquid stocks). Cells were seeded in 96-well white tissue culture-treated plates (Perkin Elmer) at 50 000 cells/well and incubated overnight at 37° C., 5% CO₂. Test compounds in DMSO at half-log dilutions were added to the cells and incubated for 30 mins. Final DMSO concentration was 0.5%. Cells were then stimulated with 200 ng/ml PMA+300 ng/ml ionomycin and incubated for 48 hours. IL-2 present in the supernatant was then measured by ELISA according to the kit instructions (ELISA MAX Deluxe Set Human IL-2, BioLegend). % inhibition was calculated for each well, using 30 μM of the commercially available MALT1 inhibitor, MLT-748, for 100% inhibition controls and no compound for 0% inhibition controls. IC₅₀ values were calculated using GraphPad Prism using 4-parameter non-linear regression curve fit.

The results of testing the Example compounds in one or more of the three in vitro assays described above are shown in Table 1.

TABLE 1
MALT1 assay data

| Example | Assay 1 | Assay 2 | Assay 3 |
|---|---|---|---|
| Number | IC₅₀ (μM) | IC₅₀ (μM) | IC₅₀ (μM) |
| 1 | 4.5 | | |
| 2 | 3.2 | | |
| 3 | 3.0 | 0.61 | |
| 4 | 2.8 | 0.40 | |
| 5 | 3.3 | 0.12 | |
| 6 | 0.7 | 0.1 | 2.1 |
| 7 | 6.9 | 1.2 | |
| 8 | 9.8 | >10 | |
| 9 | 4.6 | 2.0 | |
| 10 | 1.5 | 1.0 | |
| 11 | 4.1 | 1.2 | |
| 12 | 2.3 | 0.65 | |
| 13 | 1.3 | 1.3 | |
| 14 | 8.5 | 8.2 | |
| 15 | 1.3 | 0.44 | |
| 16 | 2.5 | 0.9 | |
| 17 | 0.50 | 1.5 | |
| 18 | 0.85 | 0.19 | 1.9 |
| 19 | 0.73 | 0.15 | 1.1 |
| 20 | 3.3 | 0.59 | |
| 21 | 2.3 | >3 | >10 |
| 22 | 4.0 | 4.7 | |
| 23 | 0.86 | 0.11 | 0.29 |
| 24 | 2.5 | 0.34 | |
| 25 | 1.06 | 0.23 | |
| 26 | 0.80 | 0.11 | 1.3 |
| 27 | 5.0 | 0.90 | |
| 28 | 7.3 | 1.1 | |
| 29 | 3.8 | >3 | |
| 30 | 1.5 | 1.5 | 0.42 |
| 31 | 3.0 | 0.26 | |
| 32 | 1.8 | 0.12 | 1.2 |
| 33 | 7.6 | 1.5 | |
| 34 | 0.97 | 0.05 | 0.29 |
| 35 | 0.73 | 0.06 | 0.17 |
| 36 | | 2.2 | 1.7 |
| 37 | 3.9 | 0.54 | |
| 38 | 0.73 | 0.12 | |
| 39 | 5.8 | 1.2 | |
| 40 | 0.34 | 0.06 | 0.3 |
| 41 | 3.9 | 1.4 | >10 |
| 42 | | 3.4 | |
| 43 | | 0.06 | 1.4 |
| 44 | | 0.33 | |
| 45 | | 0.20 | |
| 46 | | 0.03 | 0.74 |
| 47 | | 0.13 | >10 |
| 48 | | 0.08 | >10 |
| 49 | | 0.12 | 4.46 |
| 50 | | 0.24 | |
| 51 | | 0.25 | |
| 52 | | 7.6 | |
| 53 | | 0.06 | 0.39 |
| 54 | | 8.2 | |
| 55 | | 0.06 | 0.35 |
| 56 | | 5.1 | |
| 57 | | 0.17 | |
| 58 | | 1.29 | |
| 59 | | 0.82 | |
| 60 | | 3.82 | |
| 61 | | 0.021 | 0.286 |
| 62 | | 6.5 | |
| 63 | | 0.104 | |
| 64 | | 2.12 | |
| 65 | | 0.017 | |
| 66 | | 9.10 | |
| 67 | | 0.146 | |

### IL-2 Release PK/PD Model

Female C57BL/6 mice at approx. 6 weeks of age are allocated into groups based on body weight, and orally dosed with compound at time T-1 hr, in a vehicle comprising 20% HPBCD (w/v), 0.5% Tween 80 (v/v) and 10% (v/v) N,N-di-methyl acetamide in saline, at a volume of 10 mL/kg. One hour later, at TO, they are challenged with an intravenous injection of anti-CD3 antibody (Ultra-LEAF™, #100340 Biolegend), appropriately diluted in sterile PBS to dose 10 μg/mouse in a volume of 100 μL. 4 hours after aCD3 challenge the mice are anaesthetized and terminal blood samples collected from the carotid artery. A 20 μL blood sample is transferred to an EDTA tube, then diluted 1:1 with water and frozen at −20° C. until subsequent bioanalysis by LC-MS/MS. The remaining blood samples are allowed to clot for 30 minutes, centrifuged to separate serum, snap frozen and stored at −80° C. until IL-2 analysis by ELISA (Mouse IL-2 DuoSet ELISA, R&D Systems). IL-2 concentration in the serum is calculated by interpolation from a standard curve. Statistical analysis (ordinary one-way ANOVA and Dunnett's multiple comparison) is performed using GraphPad Prism.

1. A compound according to Formula I, or a pharmaceutically acceptable salt thereof: 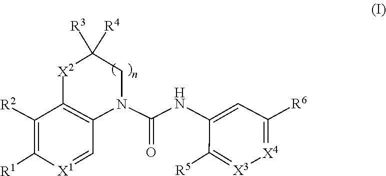
wherein:
X¹ is N or CH;
X² is O, NR⁷, or CR⁸R⁹;
X³ is N or CH;
X⁴ is N or CR¹⁰;
provided that X³ and X⁴ cannot both be N;
R¹ is selected from hydrogen, C₁₋₄alkyl, hydroxy, C₁₋₄alkoxy, C₁₋₄haloalkyl, halo, NR¹¹R¹², C₃₋₇cycloalkyl, and O—C₃₋₇cycloalkyl;
R² is selected from hydrogen, C₁₋₄alkyl, C₁₋₄haloalkyl, C₃₋₇cycloalkyl, halo, cyano, NR¹³R¹⁴, C(O)OR¹⁵, C(O)NR¹⁶R¹⁷, OR¹⁸, 4- to 7-membered heterocyclyl, 5- or 6-membered heteroaryl, and phenyl, wherein said 4- to 7-membered heterocyclyl, 5- or 6-membered heteroaryl, and phenyl are optionally substituted with one or more substituents selected from C₁₋₄alkyl, halo, and cyano;
R³ and R⁴ are independently selected from hydrogen, C₁₋₄alkyl, C₂₋₄alkenyl, C₂₋₄alkynyl, C₁₋₄haloalkyl, C₃₋₇cycloalkyl, hydroxy, C₁₋₄alkoxy, C₁₋₄haloalkoxy, halo, cyano, NR¹⁹R²⁰, 4- to 7-membered heterocyclyl, 5- or 6-membered heteroaryl, and phenyl, wherein said C₁₋₄alkyl, 4- to 7-membered heterocyclyl, 5- or 6-membered heteroaryl, and phenyl are optionally substituted with one or more substituents selected from C₁₋₄alkyl, hydroxy, C₁₋₄alkoxy, halo, and cyano; OR R³ and R⁴, together with the carbon atom to which they are attached, form an oxo group, a 4- to 7-membered heterocyclic ring, or a 3- to 6-membered cycloalkyl ring;
R⁵ is selected from hydrogen, C₁₋₄alkyl, C₁₋₄alkoxy, and C₁₋₄haloalkyl;
R⁶ is selected from hydrogen, halo, C₁₋₄alkyl, C₁₋₄alkoxy, cyano, and C₁₋₄haloalkyl;
R⁷ is hydrogen, C₁₋₄alkyl, C₃₋₇cycloalkyl, 5- or 6-membered heteroaryl or 4- to 7-membered heterocyclyl, wherein said C₁₋₄alkyl, C₃₋₇cycloalkyl, 5- or 6-membered heteroaryl, and 4- to 7-membered heterocyclyl are optionally substituted with one or more substituents selected from C₁₋₄alkyl, C₁₋₄alkoxy, halo, and cyano; OR
R⁷ and R³, together with the atoms to which they are attached, form a 5- or 6-membered heterocyclic ring, optionally substituted with one or more substituents selected from C₁₋₄alkyl, C₁₋₄alkoxy, halo, and cyano;
R⁸ and R⁹ are independently selected from hydrogen, C₁₋₄alkyl, C₂₋₄alkenyl, C₂₋₄alkynyl, C₁₋₄haloalkyl, C₃₋₇cycloalkyl, hydroxy, C₁₋₄alkoxy, C₁₋₄haloalkoxy, halo, cyano, NR²¹R²², 4- to 7-membered heterocyclyl, 5- or 6-membered heteroaryl, and phenyl, wherein said C₁₋₄alkyl, 4- to 7-membered heterocyclyl, 5- or 6-membered heteroaryl, and phenyl are optionally substituted with one or more substituents selected from C₁₋₄alkyl, C₁₋₄alkoxy, halo, and cyano; OR R⁸ and R⁹, together with the carbon atom to which they are attached, form a 4- to 7-membered heterocyclic ring, or a 3- to 6-membered cycloalkyl ring;
R¹⁰ is selected from C(O)NR²³R²⁴, C₁₋₄haloalkoxy, C₁₋₄haloalkyl, C₁₋₄alkoxy, C₃₋₇cycloalkyl, 4- to 7-membered heterocyclyl, and 5- or 6-membered heteroaryl, wherein said C₃₋₇cycloalkyl, 4- to 7-membered heterocyclyl, and 5- or 6-membered heteroaryl groups are optionally substituted with one or more substituents selected from C₁₋₄alkyl, C₁₋₃alkylene-R²⁵, hydroxy, NR²⁶R²⁷, and halo;
R¹¹ and R¹² are independently selected from hydrogen, C₁₋₄alkyl, C₃₋₇cycloalkyl, and C₁₋₄haloalkyl; or R¹¹ and R¹², together with the nitrogen atom to which they are attached, form a 4- to 7-membered heterocyclic ring;
R¹³ and R¹⁴ are independently selected from hydrogen, C₁₋₄alkyl, C₃₋₇cycloalkyl, and C₁₋₄haloalkyl; or R¹³ and R¹⁴, together with the nitrogen atom to which they are attached, form a 4- to 7-membered heterocyclic ring;
R¹⁵ is hydrogen or C₁₋₄alkyl;
R¹⁶ and R¹⁷ are independently selected from hydrogen, C₁₋₄alkyl, C₃₋₇cycloalkyl, and C₁₋₄haloalkyl; or R¹⁶ and R¹⁷, together with the nitrogen atom to which they are attached, form a 4- to 7-membered heterocyclic ring;
R¹⁸ is selected from hydrogen, C₁₋₄alkyl, C₁₋₃alkylene-R²³, C₁₋₄haloalkyl, C₃₋₇cycloalkyl, 4- to 7-membered heterocyclyl, and 5- or 6-membered heteroaryl;
R¹⁹, R²⁰, R²¹, and R²² are independently selected from hydrogen and C₁₋₄alkyl;
R²³ and R²⁴ are independently selected from hydrogen, C₁₋₄alkyl and C₃₋₇cycloalkyl;
R²⁵ is selected from OH, C₁₋₄alkoxy, C₃₋₇cycloalkyl, NR²⁸R²⁹, C(O)OR³⁰, and C(O)NR³¹R³²;
R²⁶, R²⁷, R²⁸, R²⁹, R³⁰, R³¹, and R³² are independently selected from hydrogen and C₁₋₄alkyl;
n is 0, 1, or 2.

2. The compound according to claim 1, wherein X¹ is N.

3. The compound according to claim 1 or claim 2, wherein X² is O or NR⁷.

4. The compound according to any one of claims 1 to 3, wherein X³ is N and X⁴ is CR¹⁰.

5. The compound according to any one of claims 1 to 4, wherein R¹ is selected from:
a. hydrogen, C₁₋₄alkyl, hydroxy, C₁₋₄alkoxy, and NR¹¹R¹²;
b. hydrogen, C₁₋₄alkyl, and NR¹¹R¹²;
c. hydrogen, methyl, and NH₂; or
d. hydrogen and methyl.

6. The compound according to any one of claims 1 to 5, wherein R² is selected from:
a. hydrogen, C₁₋₄alkyl, C₃₋₇cycloalkyl, halo, cyano, OR¹⁸, 5- or 6-membered heteroaryl, and phenyl, wherein said 5- or 6-membered heteroaryl, and phenyl are optionally substituted with one or more substituents selected from C₁₋₄alkyl, halo, and cyano;
b. hydrogen, C₃₋₇cycloalkyl, halo, cyano, OR¹⁸, 5- or 6-membered heteroaryl, and phenyl;
c. hydrogen, cyclopropyl, halo, cyano, methoxy, 5-membered heteroaryl, and phenyl;
d. halo, cyano, methoxy, 5-membered heteroaryl, and phenyl;
e. halo, and cyano; or
f. chloro and bromo.

7. The compound according to any one of claims 1 to 6, wherein R³ and R⁴ are independently selected from:
a. hydrogen, C₁₋₄alkyl, C₂₋₄alkynyl, C₁₋₄haloalkyl, C₃₋₇cycloalkyl, hydroxy, C₁₋₄alkoxy, halo, cyano, 4- to 7-membered heterocyclyl, 5- or 6-membered heteroaryl, and phenyl;
b. hydrogen, C₁₋₄alkyl, C₂₋₄alkynyl, C₁₋₄haloalkyl, and cyano;
c. hydrogen, C₁₋₄alkyl, ethynyl, trifluoromethyl, and cyano; or
d. hydrogen, methyl, ethyl, propyl, isopropyl, trifluoromethyl, and cyano.

8. The compound according to any one of claims 1 to 6, wherein R³ and R⁴, together with the carbon atom to which they are attached, form a 5- to 6-membered heterocyclic ring, or a 3- to 5-membered cycloalkyl ring.

9. The compound according to any one of claims 1 to 8, wherein R⁵ is hydrogen, or methyl; such as hydrogen.

10. The compound according to any one of claims 1 to 9, wherein R⁶ is selected from halo and C₁₋₄haloalkyl.

11. The compound according to claim 10, wherein R⁶ is chloro.

12. The compound according to any one of claims 1 to 11, wherein R⁷ is selected from:
a. hydrogen, C₁₋₄alkyl, and 4- to 7-membered heterocyclyl;
b. hydrogen and C₁₋₄alkyl; or
c. hydrogen and methyl.

13. The compound according to any one of claims 1 to 11, wherein R⁷ and R³, together with the atoms to which they are attached, form a 5- or 6-membered heterocyclic ring; such as a pyrrolidine or piperidine ring.

14. The compound according to any one of claims 1 to 13, wherein R⁸ and R⁹ are independently selected from hydrogen and C₁₋₄alkyl; such as hydrogen and methyl.

15. The compound according to any one of claims 1 to 13, wherein R⁸ and R⁹ are both hydrogen.

16. The compound according to any one of claims 1 to 15, wherein R¹⁰ is selected from C(O)NR²³R²⁴, C₁₋₄haloalkoxy, 4- to 7-membered heterocyclyl, and 5- or 6-membered heteroaryl, wherein said 4- to 7-membered heterocyclyl, and 5- or 6-membered heteroaryl groups are optionally substituted with one or more substituents selected from C₁₋₂alkyl, C₁₋₂alkylene-R²⁵, and halo.

17. The compound according to claim 16, wherein R¹⁰ is 5- or 6-membered heteroaryl (such as triazolyl), optionally substituted with one or more substituents selected from C₁₋₂alkyl, and C₁₋₂alkylene-R²⁵.

18. The compound according to any one of claims 1 to 17, wherein n is 0 or 1; such as 1.

19. The compound according to claim 1, wherein the compound is selected from one of the following compounds, or a pharmaceutically acceptable salt thereof:
5-Bromo-N-(5-chloro-6-(2H-1,2,3-triazol-2-yl)pyridin-3-yl)-3,4-dihydroquinoline-1 (2H)-carboxamide;
N-(6-(2H-1,2,3-Triazol-2-yl)-5-(trifluoromethyl)pyridin-3-yl)-5-bromo-3,4-dihydroquinoline-1 (2H)-carboxamide;
N-(5-Chloro-6-(2H-1,2,3-triazol-2-yl)pyridin-3-yl)-5-methoxy-3,4-dihydroquinoline-1 (2H)-carboxamide;
N-(6-(2H-1,2,3-Triazol-2-yl)-5-(trifluoromethyl)pyridin-3-yl)-8-bromo-2,3-dihydro-4H-benzo[b][1,4]oxazine-4-carboxamide;
N-(6-(2H-1,2,3-Triazol-2-yl)-5-(trifluoromethyl)pyridin-3-yl)-8-bromo-2-methyl-2,3-dihydro-4H-benzo[b][1,4]oxazine-4-carboxamide;
N-(6-(2H-1,2,3-Triazol-2-yl)-5-(trifluoromethyl)pyridin-3-yl)-8-bromo-2-cyano-2,3-dihydro-4H-benzo[b][1,4]oxazine-4-carboxamide;
N-(5-Chloro-6-(2H-1,2,3-triazol-2-yl)pyridin-3-yl)-5-cyano-3,4-dihydroquinoline-1 (2H)-carboxamide;
N-(6-(2H-1,2,3-Triazol-2-yl)-5-(trifluoromethyl)pyridin-3-yl)-5-(pyridin-3-yl)-3,4-dihydroquinoline-1 (2H)-carboxamide;
N-(6-(2H-1,2,3-Triazol-2-yl)-5-(trifluoromethyl)pyridin-3-yl)-5-(oxazol-2-yl)-3,4-dihydroquinoline-1 (2H)-carboxamide;
N-(6-(2H-1,2,3-Triazol-2-yl)-5-(trifluoromethyl)pyridin-3-yl)-5-(thiazol-2-yl)-3,4-dihydroquinoline-1 (2H)-carboxamide;
Cis-N-(6-(2H-1,2,3-Triazol-2-yl)-5-(trifluoromethyl)pyridin-3-yl)-5-bromo-3,4-dimethyl-3,4-dihydroquinoline-1 (2H)-carboxamide;
N-(6-(2H-1,2,3-Triazol-2-yl)-5-(trifluoromethyl)pyridin-3-yl)-5-phenyl-3,4-dihydroquinoline-1 (2H)-carboxamide;
8-Bromo-N-(5-chloro-6-(2H-1,2,3-triazol-2-yl)pyridin-3-yl)-2,2-dimethyl-2,3-dihydro-4H-benzo[b][1,4]oxazine-4-carboxamide;
N-(6-(2H-1,2,3-triazol-2-yl)-5-(trifluoromethyl)pyridin-3-yl)-5-(1,3,4-thiadiazol-2-yl)-3,4-dihydroquinoline-1 (2H)-carboxamide;
5-Bromo-N-(5-chloro-6-(2H-1,2,3-triazol-2-yl)pyridin-3-yl)-3,3-dimethyl-3,4-dihydroquinoline-1 (2H)-carboxamide;
5-Bromo-N-(5-chloro-6-(2H-1,2,3-triazol-2-yl)pyridin-3-yl)-4-methyl-3,4-dihydroquinoxaline-1 (2H)-carboxamide;
5-Bromo-N-(5-chloro-6-(2H-1,2,3-triazol-2-yl)pyridin-3-yl)-3,4-dihydro-1,7-naphthyridine-1 (2H)-carboxamide;
8-Bromo-N-(5-chloro-6-(2H-1,2,3-triazol-2-yl)pyridin-3-yl)-2-cyano-2-methyl-2,3-dihydro-4H-benzo[b][1,4]oxazine-4-carboxamide;
8-Bromo-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-ethynyl-2,3-dihydro-4H-benzo[b][1,4]oxazine-4-carboxamide;
8-Bromo-N-(5-chloro-6-(2H-1,2,3-triazol-2-yl)pyridin-3-yl)-2,3-dihydro-4H-pyrido[4,3-b][1,4]oxazine-4-carboxamide;
N-(5-Chloro-6-(2H-1,2,3-triazol-2-yl)pyridin-3-yl)-5-cyclopropyl-3,4-dihydro-1,7-naphthyridine-1 (2H)-carboxamide;
N-(6-(2H-1,2,3-Triazol-2-yl)-5-(trifluoromethyl)pyridin-3-yl)-8-bromo-2,2-dimethyl-2,3-dihydro-4H-benzo[b][1,4]oxazine-4-carboxamide;
N-(5-Chloro-6-(2H-1,2,3-triazol-2-yl)pyridin-3-yl)-8-cyclopropyl-2,2-dimethyl-2,3-dihydro-4H-pyrido[4,3-b][1,4]oxazine-4-carboxamide;
(S)-1-Bromo-N-(5-chloro-6-(2H-1,2,3-triazol-2-yl)pyridin-3-yl)-6a,7,8,9-tetrahydropyrido[3,4-e] pyrrolo[1,2-a]pyrazine-5 (6H)-carboxamide;
(R)-8-Bromo-N-(5-chloro-6-(2H-1,2,3-triazol-2-yl)pyridin-3-yl)-1,2-dimethyl-2,3-dihydropyrido[3,4-b]pyrazine-4 (1H)-carboxamide;
8′—Bromo-N-(5-chloro-6-(2H-1,2,3-triazol-2-yl)pyridin-3-yl)-4,5-dihydro-2H-spiro [furan-3,2′-pyrido[4,3-b][1,4]oxazine]-4′ (3′H)-carboxamide;
1-Bromo-N-(5-chloro-6-(2H-1,2,3-triazol-2-yl)pyridin-3-yl)-6,6a,7,8,9,10-hexahydro-5H-dipyrido[1,2-a: 3′,4′-e] pyrazine-5-carboxamide;
8-Bromo-N-(5-chloro-6-(2H-1,2,3-triazol-2-yl)pyridin-3-yl)-1-methyl-2,3-dihydropyrido[3,4-b]pyrazine-4 (1H)-carboxamide;
8-Bromo-N-(5-chloro-6-(2H-1,2,3-triazol-2-yl)pyridin-3-yl)-2-methyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-1,2,2-trimethyl-2,3-dihydropyrido[3,4-b]pyrazine-4 (1H)-carboxamide;
8-Chloro-N-(5-chloro-6-(2H-1,2,3-triazol-2-yl)pyridin-3-yl)-2,2-dimethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-1-(tetrahydro-2H-pyran-4-yl)-2,3-dihydropyrido[3,4-b]pyrazine-4 (1H)-carboxamide;
(R)-8-Bromo-N-(5-chloro-6-(2H-1,2,3-triazol-2-yl)pyridin-3-yl)-2-ethyl-1-methyl-2,3-dihydropyrido[3,4-b]pyrazine-4 (1H)-carboxamide;
(R)-8-Bromo-N-(5-chloro-6-(2H-1,2,3-triazol-2-yl)pyridin-3-yl)-2-isopropyl-1-methyl-2,3-dihydropyrido[3,4-b]pyrazine-4 (1H)-carboxamide;
7-Amino-8-bromo-N-(5-chloro-6-(2H-1,2,3-triazol-2-yl)pyridin-3-yl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydropyrido[3,4-b]pyrazine-4 (1H)-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-7-methyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-ethyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-isopropyl-2,3-dihydro-4H-pyrido[4,3-b][1,4]oxazine-4-carboxamide;
8-Bromo-N-(5-chloro-6-(2H-1,2,3-triazol-2-yl)pyridin-3-yl)-2-cyclopropyl-2,3-dihydro-4H-pyrido[4,3-b][1,4]oxazine-4-carboxamide;
N-(5-Chloro-6-(2H-1,2,3-triazol-2-yl)pyridin-3-yl)-8-cyano-7-methyl-2-(trifluoromethyl)-2,3-dihydro-4H-pyrido[4,3-b][1,4]oxazine-4-carboxamide;
N-(5-Chloro-6-(2H-1,2,3-triazol-2-yl)pyridin-3-yl)-8-cyano-2-(trifluoromethyl)-2,3-dihydro-4H-pyrido[4,3-b][1,4]oxazine-4-carboxamide;
N-(5-Chloro-6-(2H-1,2,3-triazol-2-yl)pyridin-3-yl)-8-methoxy-2-(trifluoromethyl)-2,3-dihydro-4H-pyrido[4,3-b][1,4]oxazine-4-carboxamide;
5-Bromo-N-(5-chloro-6-(2H-1,2,3-triazol-2-yl)pyridin-3-yl)-3-cyano-3,4-dihydro-1,7-naphthyridine-1 (2H)-carboxamide;
N-(6-(2H-1,2,3-triazol-2-yl)-5-(trifluoromethyl)pyridin-3-yl)-8-cyano-7-methyl-2-(trifluoromethyl)-2,3-dihydro-4H-pyrido[4,3-b][1,4]oxazine-4-carboxamide;
8-cyano-N-(5-cyano-6-(2H-1,2,3-triazol-2-yl)pyridin-3-yl)-7-methyl-2-(trifluoromethyl)-2,3-dihydro-4H-pyrido[4,3-b][1,4]oxazine-4-carboxamide;
8-cyano-N-(6-(cyclopropyl(methyl) carbamoyl)-5-(difluoromethyl)pyridin-3-yl)-7-methyl-2-(trifluoromethyl)-2,3-dihydro-4H-pyrido[4,3-b][1,4]oxazine-4-carboxamide;
8-cyano-7-methyl-2-(trifluoromethyl)-N-(2-(trifluoromethyl)pyridin-4-yl)-2,3-dihydro-4H-pyrido[4,3-b][1,4]oxazine-4-carboxamide; and
N-(5-chloro-6-(difluoromethoxy)pyridin-3-yl)-8-cyano-7-methyl-2-(trifluoromethyl)-2,3-dihydro-4H-pyrido[4,3-b][1,4]oxazine-4-carboxamide.

20. A pharmaceutical composition comprising a compound according to any one of claims 1 to 19, or a pharmaceutically acceptable salt thereof, and one or more pharmaceutically acceptable excipients.

21. A compound according to any one of claims 1 to 19, or a pharmaceutically acceptable salt thereof, or a pharmaceutical composition according to claim 20, for use in therapy.

22. A compound according to any one of claims 1 to 19, or a pharmaceutically acceptable salt thereof, or a pharmaceutical composition according to claim 20, for use in the treatment of diseases or disorders mediated by MALT1.

23. A compound according to any one of claims 1 to 19, or a pharmaceutically acceptable salt thereof, or a pharmaceutical composition according to claim 20, for use in the treatment of non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenstrom macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis.

24. A method of treating a disease or disorders mediated by MALT1, said method comprising administering to a subject in need of such treatment a therapeutically effective amount of a compound according to any one of claims 1 to 19, or a pharmaceutically acceptable salt thereof, or a pharmaceutical composition according to claim 20.

25. A method of treating non-Hodgkin's lymphoma (IN-IL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukaemia (CLL), small lymphocytic lymphoma (SLL), Waldenstrom macroglobulinemia, lymphoblastic T cell leukaemia, chronic myelogenous leukaemia (CML), hairy-cell leukaemia, acute lymphoblastic T cell leukaemia, plasmacytoma, immunoblastic large cell leukaemia, megakaryoblastic leukaemia, acute megakaryocytic leukaemia, promyelocytic leukaemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, oesophageal cancer, salivary gland cancer, nasopharyngeal cancer, buccal cancer, cancer of the mouth, GIST (gastrointestinal stromal tumour), arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, or polymyositis, said method comprising administering to a subject in need of such treatment a therapeutically effective amount of a compound according to any one of claims 1 to 19, or a pharmaceutically acceptable salt thereof, or a pharmaceutical composition according to claim 20.
